(19) United States
(12) Patent Application Publication
(10) Pub. No.: US 2022/0110963 A1
(43) Pub. Date: Apr. 14, 2022

(54) REVERSE TRANSCRIPTASE BLOCKING AGENTS AND METHODS OF USING THE SAME

(71) Applicant: The General Hospital Corporation, Boston, MA (US)

(72) Inventors: David T. Ting, Dover, MA (US); Mihir S. Rajurkar, Medford, MA (US)

(21) Appl. No.: 17/420,550

(22) PCT Filed: Jan. 2, 2020
(86) PCT No.: PCT/US2020/012085

§ 371 Date: Jul. 2, 2021

Related U.S. Application Data
(60) Provisional application No. 62/787,709, filed on Jan. 2, 2019.

(51) Int. Cl. A61K 31/7072; A61K 31/7105; A61K 31/506; A61K 31/52; A61K 31/675; A61K 31/505; A61K 31/522; A61K 38/46; A61K 31/536; A61K 31/4439; A61K 31/4162; A61K 39/395; A61K 31/706; A61P 35/00

(52) U.S. Cl. CPC A61K 31/7072; A61K 31/7105; A61K 31/506; A61K 31/52; A61K 31/675; A61K 31/505; A61P 35/00; A61K 38/465; A61K 31/536; A61K 31/4439; A61K 31/4162; A61K 39/3955; A61K 31/706; A61K 31/522

(57) ABSTRACT

Provided herein are reverse transcriptase (RT) blocking agents and methods of using the same for the treatment of cancer (e.g., an epithelial cancer) in a subject in need thereof.

### CLAIM OF PRIORITY

This application claims the benefit of U.S. Provisional patent application Ser. No. 62/787,709, filed on Jan. 2, 2019. The entire contents of the foregoing are hereby incorporated by reference.

### FEDERALLY SPONSORED RESEARCH OR DEVELOPMENT

This invention was made with Government support under Grant No. W81XWH-13-1-0237, awarded by the U.S. Department of Defense. The Government has certain rights in the invention.

### TECHNICAL FIELD

The present invention relates to methods for treating cancer, e.g., cancer of epithelial origin, in a subject by administering a reverse transcriptase (e.g., HERV-K RT) blocking agent.

### BACKGROUND

The human genome has a high percentage of non-protein coding genomic regions organized as tandemly repeated sequences. These genomic regions are normally transcriptionally silent, but can be transcribed in cancer cells. For example, the pericentrometric human satellite II (HSATII) sequence has been shown to be overexpressed in epithelial cancers (e.g., pancreatic cancer) yet silenced in normal cells (see Prosser et al. J. Mol. Biol. 187(2): 145-55 (1986); Warburton et al. (2008) BMC Genomics 9: 533; and Ting et al. (2011) Science 331(6017): 593-6; International Publication No. WO 2012/0481131).

### SUMMARY

Disclosed herein is a method of treating a subject with cancer comprising administering to the subject a reverse transcriptase inhibitor (RTI) and a DNA hypomethylating agent. In some embodiments, the RTI is selected from zidovudine (ZDV), didanosine (ddI), stavudine (d4T), zalcitabine (DDC), lamivudine (3TC), abacavir (ABC), tenofovir disoproxil (TDF), emtricitabine (FTC), etravirine lobucavir, entecavir (ETV), apricitabine, censavudine, dexelvucitabine, alovudine, amdoxovir, elvucitabine, racivir, and stampidine. In some embodiments, the RTI is 3TC.

As shown herein, the inhibition of HSATII reverse transcription inhibition using reverse transcriptase inhibitors induces cell death (e.g., via necroptosis) in cancer cell lines grown as 3D tumorspheres that overexpress HSATII. The human endogenous retrovirus-K reverse transcriptase (HERV-K RT) appears to be the reverse transcriptase responsible for HSATII reverse transcription. Thus, provided herein are methods of treating cancer (e.g., an epithelial cancer) by specifically targeting HERV-K RT. In addition, as shown herein, a combination of (i) a DNA hypomethylating agent and (ii) reverse transcriptase inhibitor (e.g., a nucleoside analog reverse transcriptase inhibitor, a nucleotide analog reverse transcriptase inhibitor, non-nucleoside reverse transcriptase inhibitor, or a combination thereof), can be used to treat cancers.

In one aspect, provided herein is a method of treating a subject with cancer in a subject in need thereof, wherein the cancer expresses high levels of HSATII RNA, the method comprising administering to the subject a therapeutically effective amount of a HERV-K reverse transcriptase (HERV-K RT) blocking agent.

In some embodiments, the HERV-K RT blocking agent is an inhibitory nucleic acid. In some embodiments, the HERV-K RT blocking agent is selected from the group consisting of a locked nucleic acid (LNA) molecule, a short hairpin RNA (shRNA) molecule, a small inhibitory RNA (siRNA) molecule, an antisense nucleic acid molecule, a peptide nucleic acid molecule, a morpholino, and a ribozyme.

In some embodiments, the HERV-K RT blocking agent comprises a zinc finger nuclease system, a transcription activator-like effector nuclease (TALEN) system, a meganuclease system, a Cpf1 nuclease system, a CRISPR/Cas9 system, or a CRISPR/Cas13 nuclease system.

In some embodiments, the HERV-K RT blocking agent is selected from the group consisting of a nucleoside analog reverse transcriptase inhibitor, a nucleotide analog reverse transcriptase inhibitor, non-nucleoside reverse transcriptase inhibitor, and a combination thereof. In some embodiments, the nucleoside analog reverse transcriptase inhibitor comprises lamivudine, abacavir, zidovudine, emtricitabine, didanosine, stavudine, entecavir, apricitabine, censavudine, zalcitabine, dexelvucitabine, amdoxovir, elvucitabine, festinavir, racivir, stampidine, or a combination thereof. In some embodiments, the non-nucleoside reverse transcriptase inhibitor comprises lersivirine, rilpivirine, efavirenz, etravirine, doravirine, dapivirine, or a combination thereof. In some embodiments, the nucleotide analog reverse transcriptase inhibitor comprises tenofovir alafenamide fumarate, tenofovir disoproxil fumarate, adefovir, or a combination thereof. In some embodiments, the HERV-K RT blocking agent is a cytidine analog or a guanosine analog.

In some embodiments, the HERV-K RT blocking agent comprises an anti-HERV-K RT antibody.

In some embodiments, the administering results in a reduction in tumor burden in the subject. In some embodiments, the administering results in the death of a cancer cell in the subject via necroptosis. In some embodiments, the cancer is an epithelial cancer. In some embodiments, the epithelial cancer is pancreatic cancer, colorectal cancer, breast cancer, prostate cancer, renal cancer, ovarian cancer, or lung cancer. In some embodiments, the colorectal cancer comprises microsatellite instable (MSI) colorectal cancer or microsatellite stable (MSS) colorectal cancer.

In some embodiments, the method further comprises administering an additional therapeutic agent to the subject. In some embodiments, the additional therapeutic agent is an immunotherapy agent selected from the group consisting of an anti-PD-1 antibody, an anti-PD-L1 antibody, an anti-CD137 antibody, an anti-CTLA4 antibody, an anti-CD40 antibody, an anti-IL10 antibody, an anti-TGF-β antibody, and an anti-IL-6 antibody. In some embodiments, the method further comprises administering a DNA hypomethylating agent to the subject.

In some embodiments, the method comprises: detecting a level of HSATII RNA in a sample from the cancer; comparing the level of HSATII RNA in the sample to a reference level; identifying a subject who has cancer that has levels of HSATII RNA above the reference level; and selecting the identified subject for treatment with the HERV-K reverse transcriptase (HERV-K RT) blocking agent.

In some embodiments, the cancer comprises a mutation in tumor protein p53 (TP53).

In some embodiments, the method comprises: detecting a level of TP53 in a sample from the cancer; comparing the level of TP53 protein in the sample to a reference level; identifying a subject who has cancer that has levels of TP53 protein below the reference level; and selecting the identified subject for treatment with the HERV-K reverse transcriptase (HERV-K RT) blocking agent. In some embodiments, the method further comprises administering a DNA hypomethylating agent to the subject.

In some embodiments, the DNA hypomethylating agent is azacytidine, decitabine, cladribine, or a combination thereof.

In some embodiments, the method comprises: detecting a mutation in a TP53 allele in a sample from the cancer; and selecting the subject for treatment with the HERV-K RT blocking agent. In some embodiments, detecting a mutation in a TP53 allele in a sample from the cancer comprises: determining a TP53 sequence in the sample and comparing the sequence to a reference sequence; identifying a subject who has cancer that has a mutation in a TP53 allele; and selecting the identified subject for treatment with the HERV-K RT blocking agent.

In some embodiments, detecting a mutation in a TP53 allele in a sample from the cancer comprises: contacting the sample with one or more probes that specifically detect a mutation in a TP53 allele; detecting binding of the one or more probes to the sample, thereby detecting the presence of a mutation in a TP53 allele in the cancer; identifying a subject who has cancer that has a mutation in a TP53 allele; and selecting the identified subject for treatment with the HERV-K RT blocking agent.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 shows that HSATII expression is linked to growth in 3D tumorspheres and not in standard 2D culture. DLD1, HCT8, HCT116, HT29, and SW620 cell lines expressed HSATII RNA when grown as 3D tumorspheres, but not when grown under adherent 2D cell culture conditions. In contrast, the semi-adherent cell line COL0205 expressed HSATII RNA when cultured using standard 2D adherent cell culture plates because this cell line grows in a 3D architecture even when cultured using standard 2D culture conditions.

FIG. 2 shows that direct targeting of HSATII RNA with locked nucleic acids (LNAs) increases HSAT II RNA levels in COL0205 cells. Neg 1-Neg 6 refers to COL0205 cells treated with a scrambled non-specific LNA for a period of 1 to 6 days. Sat 1-Sat6 refers to COL0205 cells treated with an HSATII-specific LNA for a period of 1 to 6 days. Bar graph represents HSATII RNA levels. HSATII levels peak after 2 to 3 days of treatment with HSATII-specific LNAs.

FIGS. 3A-3D show that LNAs targeting HSATII induce cell death in microsatellite stable (MSS) colorectal cancer cells but not microsatellite instable (MSI) colorectal cancer cells. HSATII-specific LNA (HSATII LNA2) induces cell death in the SW620 and DLD1 cell lines (MSS), but not in the HCT8 and HCT116 cell lines (MSI) when grown as 3D tumorspheres. A non-specific scrambled LNA (“scrambled”) has no effect.

FIG. 4 shows that nucleoside reverse transcriptase inhibitors (NRTIs) increase HSATII RNA accumulation in the HCT116 colorectal cancer cell line. Total RNA from HCT116 cells grown in 2D culture and from mouse xenografts treated with vehicle control (“Cont”) or the NRTI 2′,3′-dideoxycytidine (“ddC”) were subjected to Northern blot analysis. HSATII was detected using a ³²P-labeled DNA oligo: anti-HSATII S, 5′-CATTCGATTCCATTCGATGAT-3′ (SEQ ID NO: 3). Total RNA was either not treated (“NT”), treated with DNaseI (“DNase I”), or treated with RNase A (“RNase A”) before analysis. An increase of total HSATII signal was detected in ddC-treated xenograft extracted RNA as compared to control, which was abrogated in samples treated with RNase A, indicating that the increased signal is from HSATII RNA.

FIG. 5 shows that NRTIs have no effect in adherent CRC cells (2D cultured). DMSO=dimethyl sulfoxide; ddA=2′,3′-dideoxyadenosine (ddA); Teno=tenofovir; d4T=stavudine; ZDV=zidovudine; ddC=2′,3′-dideoxycytidine (zalcitabine); FTC=emtricitabine; 3TC=lamivudine; ABC=abacavir; ETV=entecavir.

FIG. 6 shows the efficacy of various NRTIs in colorectal cancer cell lines grown as tumorspheres (3D). DMSO=dimethyl sulfoxide; ddA=2′,3′-dideoxyadenosine; Teno=tenofovir; d4T=stavudine; ZDV=zidovudine; ddC=2′,3′-dideoxycytidine (zalcitabine); FTC=emtricitabine; 3TC=lamivudine; ABC=abacavir; ETV=entecavir.

FIG. 7 is a schematic showing a human chromosome showing the relative location of telomeres (top and bottom of schematic), which are reverse transcribed by telomerase reverse transcriptase, LINE-1 retrotransposons which are reverse transcribed by LINE-1 reverse transcriptase, and HSATII, which is believed to be reverse transcribed by the HERV-K reverse transcriptase. HERV-K sequences are found in the same location as HSATII, and there is a shared 5 base pair similarity between the HSATII consensus sequence and the repetitive sequence found in the HERV-K 5′ LTR and 3′ LTRs. LTRs (Long Terminal Repeats) are critical determinants of retroviral integration in the genome.

FIG. 8 shows that HERV-K expression is linked with HSATII copy number gain.

FIGS. 9A and 9B show that innate immune/pattern recognition, and necroptotic pathways are preferentially enriched in cancer cells grown as tumorspheres vs as adherent cells (standard 2D culture), as opposed to apoptosis, which is preferentially enriched in 2D.

FIG. 10 shows that transfection with in vitro synthesized ectopic HSATII RNA causes cell death in adherent grown cells when reverse transcriptase activity is blocked using NRTIs. Cell swelling is indicative of necroptosis.

FIGS. 11A-11D show that RIPK3 knockdown using three different shRNAs rescues NRTI induced cell death in SW620 cells grown as tumorspheres (3D). DMSO=dimethyl sulfoxide; ddA=2′,3′-dideoxyadenosine; Teno=tenofovir; d4T=stavudine; ZDV=zidovudine; ddC=2′,3′-dideoxycytidine (zalcitabine); FTC=emtricitabine; 3TC=lamivudine; ABC=abacavir; ETV=entecavir.

FIGS. 12A and 12B show that pharmacological necroptosis inhibition using necrostatin-1 (10 μM) rescues cell death induced by either an HSATII-specific LNA (FIG. 12A) or the NRTI 2′,3′-dideoxycytidine (FIG. 12B).

FIGS. 13A and 13B show that treatment of colorectal cancer cells grown as tumorspheres with the DNA hypomethylating agent 5′-azacytidine (Aza) in combination with an NRTI exhibits a synergistic effect on cell death induction. DMSO=dimethyl sulfoxide; ddA=2′,3′-dideoxyadenosine; Teno=tenofovir; d4T=stavudine; ZDV=zidovudine; ddC=2′,3′-dideoxycytidine (zalcitabine); FTC=emtricitabine; 3TC=lamivudine; ABC=abacavir; ETV=entecavir.

FIG. 14 shows that treatment of colorectal cancer cells grown as tumorspheres with the DNA hypomethylating agent 5′-azacytidine (Aza) sensitizes the cells to NRTI-induced cell death. DMSO=dimethyl sulfoxide; ddA=2′,3′-dideoxyadenosine; Teno=tenofovir; d4T=stavudine; ZDV=zidovudine; ddC=2′,3′-dideoxycytidine (zalcitabine); FTC=emtricitabine; 3TC=lamivudine; ABC=abacavir; ETV=entecavir.

FIG. 15 shows that treatment with either the NRTI lamivudine (3TC) alone, or 3TC in combination with 5′-azacytidine (Aza) induces tumor regression in a SW620 xenograft model of colorectal cancer.

FIG. 16 shows that treatment with 3TC in combination with 5′-azacytidine (Aza) induces tumor regression in a HCT116 xenograft tumor model of colorectal cancer.

FIGS. 17A and 17B show that the presence of HSATII RNA positively correlates with intratumoral macrophages in human colorectal cancer tissues.

FIG. 18 shows that the presence of HSATII RNA anti-correlates with CD8⁺ T cells in human colorectal and pancreatic cancer tissue.

FIG. 19 is a heat map showing the probability of interaction between each of the endogenous human reverse transcriptases HERV-K RT, hTERT, LINE1-RT, or the exogenous viral reverse transcriptases HIV-1 RT, HBV Pol, or HTLV RT, and a repeat RNA nucleic acid element (y-axis).

FIG. 20 shows HCT-8 and DLD1 colon cancer cell lines with dCAS9-KRAB+control gRNA or HERVK gRNA grown in 3D tumorsphere culture.

FIGS. 21A-21G shows TP53 linked with regulation of repeat RNA expression and differential sensitivity to Repeatome drugs. FIGS. 21A-21B show TP53-mutant or wildtype CRC cell lines are treated with a panel of clinically approved NRTIs (5 μM) alone (FIG. 21A) or with 300 nM AZA (FIG. 21). Cell viability is measured using Cell-titer Glow assay at treatment day 7, and represented as Relative Tumoursphere (%) normalized to DMSO. FIG. 21C shows TP53 IP-seq conducted using an antibody targeting TP53 in TP53-wildtype (TP53-WT: HCT116, HCT8) and TP53-mutant (TP53-Mut: SW620, DLD1) cell lines with significant loss of TP53 enrichment in mutant cell lines at repetitive elements shown (Legend: Scaled-Log 10(RPM) counts normalized to IgG input control). FIG. 21D shows distribution of significantly enriched repeats (FDR<0.2) from TP53 ChIP-seq in TP53-WT and TP53-Mut cell lines (SAT=satellite, L1=LINE1, ERV=endogenous retrovirus, LTR=long terminal repeat, DNA Tran=DNA Transposon, SVA RT=SVA retrotransposon. FIG. 21E shows HSATII RNA across TP53-WT or TP53-Mut CRC tumourspheres, as measured by RNA in situ hybridization (RNA-ISH). Signal is quantified as ratio of HSATII-positive area in tumourspheres across 20 fields. p-value across four samples is calculated using one-way ANOVA. FIG. 21F shows HCT8 tumoursphere viability with TP53 knockdown (shTP53) or non-target scrambled shRNA (shNT) treated with NRTI ddC. *** indicates p<0.001 using two tailed student t-test. FIG. 21G shows tumoursphere growth over 4-6 days treated with scrambled or HSATII targeted LNA. *** indicates p<0.001 and ** indicates p<0.01 using two-way ANOVA.

FIG. 22 shows NRTI treatment in two-dimensional culture of colorectal cancer cells.

FIG. 23 shows NRTI treatment in three-dimensional culture of colorectal cancer cells.

FIGS. 24A-24H show Repeatome modulation correlated with chromatin factors and associated with necroptotic cell death. FIGS. 24A-24C shows transcriptional changes in SW620, DLD1, HCT8, and HCT116 CRC tumourspheres treated with 5 M of NRTI Lamivudine (3TC), 300 nM AZA, both agents, or DMSO control for 24 hrs followed by RNA-seq analysis. FIG. 24A shows consensus repeat RNA expression and chromatin genes shown with LOG 2 fold change in treated versus DMSO control. * indicates FDR<0.05. FIG. 24B shows SAT repeats with highest fold change in treated versus DMSO. FIG. 24C shows GSEA of coding genes between treated and DMSO control showing normalized enrichment score (y-axis) of chromatin related gene sets with an FDR<0.0001. FIGS. 24D and 24E show correlation analysis of coding genes with HSATII demonstrating (FIG. 24D) GSEA normalized enrichment score of chromatin factors, and enrichment plot for GO:Chromatin and (FIG. 24E) scatter plot of HSATII expression with highly anti-correlated chromatin factors. FIG. 24F shows GSEA on differentially expressed genes in SW620, DLD1, HCT8, and HCT116 CRC tumorspheres treated for 7 days with 5 μM of NRTI Lamivudine (3TC), 300 nM AZA, or both agents compared to DMSO control indicates significant enrichment of genes involved in inflammatory response, and cytokine activity (HALLMARK INFLAMMATORY RESPONSE; GO:CYTOKINE ACTIVITY). FIG. 24G shows GSEA on differentially expressed genes in TP53 mutant (SW620, DLD1) tumourspheres treated with 5 μM of 3TC for 7 days indicates significant enrichment of genes involved in Interferon Gamma Response (HALLMARK INTERFERON GAMMA RESPONSE. FIGS. 24G and 24H show tumoursphere viability in response to NRTIs (5 μM) in SW620 cells expressing non-targeting shRNA (shNT), or shRNAs targeting necroptosis effector RIPK3. shRNA knockdown of RIPK3 rescues NRTI toxicity. *** indicates p<0.001 using student's two tailed t-test.

FIG. 25 shows gene set enrichment analysis (GSEA) of coding genes.

FIGS. 26A-26B show relative viability after treatment of SW620 cells, DLD1 cells, HCT8 cells, and HCT116 cells with DMSO or Necrostatin-1.

FIGS. 27A-27H show Repeatome targeting drugs with in vivo efficacy and complementary effects with cytotoxic therapies. FIG. 27A shows luciferase-expressing SW620 cells were subcutaneously implanted in immunocompromised Nude mice, and grown for 2 weeks after which, mice were randomized and divided into four treatment arms: Control (PBS), 3TC, AZA, and 3TC+AZA. Drugs were administered 3 times a week at a dosage of 50 mg/kg 3TC, and 0.75 mg/kg AZA. FIG. 27A shows tumor volume was measured using IVIS imaging every 5 days. Graph represents relative luminescence units (RLU) normalized to Day 0. **** indicates p<0.0001, calculated using two-way ANOVA test. FIG. 27C shows SW620 tumors separated into Repeat-HIGH and Repeat-LOW tumors after treatment based on median HSATII, the most highly expressed repeat RNA. FIG. 27D shows gene set enrichment analysis revealed chromatin regulatory genes as strongly anti-correlated with HSATII RNA. FIG. 27E shows a heat map providing expression of top anti-correlated coding genes (vs HSATII) in Control, and treated (Repeat-HIGH and Repeat-LOW) tumors. Histones and chromatin modifiers are expressed at lower levels in Repeat-HIGH tumors compared to Repeat-LOW tumors. RNA-seq data is represented as normalized LOG 2 reads per million (RPM). FIG. 27F shows HSATII expression in HCT8 tumourspheres grown in the presence of DMSO (control), or 50 μM 5FU+1.25 μM Oxaliplatin for 2 weeks. Scale bar=20 pun. HSATII RNA-ISH in DLD1, SW620, HCT8, and HCT116 tumourspheres treated with DMSO or 50 μM 5FU+1.25 μM1 Oxaliplatin for 2 weeks quantified using Visiopharm software. FIG. 27G shows 1HSATII RNA on human colorectal cancer tumors from untreated patients (Left), and patients who received neoadjuvant chemoradiation (Right). Scale bar=20 μm, HSATII RNA levels quantified using Visiopharn digital image analysis. FIG. 27H shows colorectal cancer tumourspheres treated with either DMSO (control) or 5 μM 3TC for 10 days in the presence of 50 μM 5FU+1.25 μM Oxaliplatin For FIGS. 27F-27H shows student's two tailed t-test used for comparisons with statistical significance of * p<0.05, ** p<0.01, * ** p<0.001, **** p<0.0001.

FIG. 28 shows NRTI and AZA treatment in three-dimensional culture of colorectal cancer cells.

FIG. 29A shows gene set enrichment analysis in pre-ranked vs HSATII in SW620 cells. FIG. 29B shows a heat map analysis of top anti-correlated genes in control vs treated groups of SW620 cells. FIG. 29C shows xenograft tumor growth after injection with HCT116 cells. FIG. 29D shows a heat map analysis of repeat RNAs in control vs treated groups of HCT116 cells. FIG. 29E shows a heat map analysis of epigenetic regulators in control vs treated groups of HCT116 cells.

FIGS. 30A-30G shows clinical trial of NRTI effects on TP53 mutant metastatic colorectal cancers. FIG. 30A shows a schema of Phase II clinical trial of 3TC single agent in TP53 mutant CRC with correlative blood, biopsy, and staging scans. FIG. 30B shows a swimmer plot of time on 3TC treatment (x-axis days) for initial 24 patients enrolled (y-axis patient ID). * indicates patients on treatment at the time of data analysis. FIG. 30C shows a best serum Cancer Embryonic Antigen (CEA) response in patients on the clinical trial. Patients with stable disease (7, 8, 11, 15, and 20) had unchanged or decrease in serum CEA levels, while most patients with progression had increased CEA. One patient (21) had mixed response to treatment and had a drop in serum CEA levels. FIGS. 30D and 30E show a RNA-seq differential expression analysis between progressive disease (PD) and stable disease (SD) shown as a volcano plot for (FIG. 30D) coding genes and (FIG. 30E) repeat R1NAs. Y-axis is Log(FDR) and x-axis is Log 2(Fold change). FIG. 30F shows an RNA-seq for different classes of repeats differentially expressed in SD compared to PD. RNA expression shown as Log 2(RPM). FIG. 30G shows chromatin factors correlated with HSATII expression with significant enrichment by GSEA (NES 4.13, FDR<. 0.01).

FIGS. 31A-31F show L1 expression in Barrett's Esophagus. FIG. 31A shows Het1A cells untreated or exposed to DCA stained with CDX2 and L1 RNA-ISH with fluorescent intensity quantitation (FIGS. 31B-31C). FIG. 31D shows Het1A cells with shNT and shTP53 stained with CDX2 and L1 RNA-ISH with quantitation (FIG. 31E). Fluorescent microscope images at 400× magnification. BE biopsy sample with high-grade dysplasia stained for L1 RNA-ISH with representative image at 400× magnification of L1 High (FIG. 31F). All plots with mean (bar) and t-test p-value (**<0.05, ***<0.0005).

### DETAILED DESCRIPTION

The methods described herein can include the administration of an agent (e.g., a HERV-K reverse transcriptase (HERV-K RT) blocking agent) to a subject to treat cancer, e.g., solid tumors of epithelial origin, e.g., pancreatic, lung, breast, prostate, renal, ovarian, or colorectal cancer, in the subject.

As used herein, the term “hyperproliferative” refer to cells having the capacity for autonomous growth, i.e., an abnormal state or condition characterized by rapidly proliferating cell growth. Hyperproliferative disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness. A “tumor” is an abnormal growth of hyperproliferative cells. “Cancer” refers to pathologic disease states, e.g., characterized by malignant tumor growth. In some embodiments, the methods described herein result in the inhibition of tumor cell proliferation in a subject. In some embodiments, the methods described herein result in increased tumor cell death or killing in the subject. In some embodiments, the methods described herein result in the inhibition of a rate of tumor cell growth or metastasis. In some embodiments, the methods described herein result in a reduction in the size of a tumor in a subject. In some embodiments, the methods described herein result a reduction in tumor burden in a subject. In some embodiments, the methods described herein result in a reduction in the number of metastases in a subject.

As used herein, the term “sample” refers to any biological sample obtained from a subject, cell line, tissue, or other source of cells (e.g., blood). Non-limiting sources of a sample for use in the present disclosure include solid tissue, biopsy aspirates, ascites, fluidic extracts, blood, plasma, serum, spinal fluid, lymph fluid, the external sections of the skin, respiratory, intestinal, and genitourinary tracts, tears, saliva, milk, tumors, organs, cell cultures and/or cell culture constituents, for example.

As disclosed herein, a “reference sample” can be used to correlate and compare the results obtained using various methods of the disclosure from a test sample. Reference samples can be cells (e.g., cell lines, cell pellets) or tissue. The amount of a transcript (e.g., LINE-1) in the “reference sample” may be an absolute or relative amount, a range of amount, a minimum and/or maximum amount, a mean amount, and/or a median amount of the transcript. The diagnostic methods of the disclosure involve a comparison between expression of a gene, transcript, or protein of interest in a test sample and a “reference value.” In some embodiments, the reference value is the expression of the gene, transcript, or protein of interest in a reference sample. A reference value may be a predetermined value and may be determined from reference samples (e.g., control biological samples) tested in parallel with the test samples. A reference value can be a single cut-off value, such as a median or mean or a range of values, such as a confidence interval. In some embodiments, the reference sample is a sample from a healthy tissue, in particular a corresponding tissue which is not affected by a neurodegenerative disorder. These types of reference samples are referred to as negative control samples.

As used herein, treating includes “prophylactic treatment” which means reducing the incidence of or preventing (i.e., reducing risk of) a sign or symptom of a disease in a subject at risk for the disease, and “therapeutic treatment”, which means reducing signs or symptoms of a disease, reducing progression of a disease, reducing severity of a disease, in a subject diagnosed with the disease.

The presence of cancer, e.g., solid tumors of epithelial origin, e.g., as defined by the ICD-O (International Classification of Diseases—Oncology) code (revision 3), section (8010-8790), e.g., early stage cancer, is associated with the presence of a massive levels of satellite due to increased transcription and processing of satellite repeats in epithelial cancer cells (see, e.g., Ting et al. (2011) Science 331(6017): 593-6; Bersani et al. (2015) Proc. Natl. Acad. Sci. U.S.A. 112(49): 15148-53; and U.S. Publication No. 2017/0198288 A1, the entire contents of each of which are expressly incorporated herein by reference). Applicants have identified the HERV-K RT as the reverse transcriptase responsible for the increased levels of the HSATII RNA in cancer cells. As described herein, inhibition of the HERV-K RT unexpectedly induces cell death in cancer cells having high levels of HSATII RNA. Thus, the methods described herein can include the interference of a HERV-K RT by administering a HERV-K RT inhibitor.

In some embodiments, the methods described herein are used in treating a subject who has a cancer of epithelial origin (i.e., an epithelial cancer). Cancers of epithelial origin can include pancreatic cancer (e.g., pancreatic adenocarcinoma), lung cancer (e.g., non-small cell lung carcinoma or small cell lung carcinoma), prostate cancer, breast cancer, renal cancer, ovarian cancer, or colon cancer. Satellites have also been shown to be elevated in preneoplastic or early cancer lesions including intraductal papillary mucinous neoplasm (IPMN), pancreatic intraepithelial neoplasia (PanIN), ductal carcinoma in situ (DCIS), Barrett's Esophagus (see e.g., Sharma (2009) N. Engl. J Med. 361(26): 2548-56; erratum in: N Engl J Med. 362(15): 1450). Thus, the methods can be used to potentially treat early preneoplastic cancers as a means to prevent the development of invasive cancer. In some embodiments, the cancer is a microsatellite instable (MSI) cancer (e.g., microsatellite stable colorectal cancer). In some embodiments, the cancer is a microsatellite stable cancer (e.g., MSS colorectal cancer).

As used herein, high levels of satellite RNA means levels above a reference level or threshold, e.g., a reference that represents a statistically determined threshold above which cancer can be diagnosed or treated using a method described herein; suitable reference levels can be determined by methods known in the art. In some embodiments, the methods include detecting the presence of high levels of satellite RNA, e.g., levels of satellite RNA above a threshold, in a sample from the subject, e.g., a biopsy sample comprising tumor cells or tumor tissue from the subject. Levels of satellite RNA can be determined by any method known in the art, including Northern blot, RNA in situ hybridization (RNA ISH), RNA expression assays, e.g., microarray analysis, RT-PCR, deep sequencing, cloning, Northern blot, and quantitative real time polymerase chain reaction (qRT-PCR) (see International Publication No. WO 2012/048113, which is incorporated by reference herein in its entirety). In some embodiments, in place of detecting high levels of satellite RNA, the methods include detecting copy number of satellite RNA. An increase in copy number as compared to a normal cell, and/or an increase in levels of satellite RNA, indicates that the cancer is susceptible to a treatment described herein. Thus, the methods can include detecting and/or identifying a cancer that has high levels of satellite RNA and/or an increased HSATII copy number, and/or selecting a subject who has a cancer with high levels of satellite RNA and/or an increased satellite RNA copy number, for treatment with a method described herein. See US 2017-0356054 A1 and WO2012048113A2, each of which is incorporated by reference in its entirety.

As used herein, “high levels of HSATII RNA” means levels above a reference level or threshold, e.g., a reference that represents a statistically determined threshold above which cancer can be diagnosed or treated using a method described herein; suitable reference levels can be determined by methods known in the art. In some embodiments, the methods include detecting the presence of high levels of HSATII RNA, e.g., levels of HSATII RNA above a threshold, in a sample from the subject, e.g., a biopsy sample comprising tumor cells or tumor tissue from the subject. Levels of HSATII RNA can be determined by any method known in the art, including Northern blot, RNA in situ hybridization (RNA ISH), RNA expression assays, e.g., microarray analysis, RT-PCR, deep sequencing, cloning, Northern blot, and quantitative real time polymerase chain reaction (qRT-PCR) (see International Publication No. WO 2012/048113, which is incorporated by reference herein in its entirety). In some embodiments, in place of detecting high levels of HSATII RNA, the methods include detecting copy number of HSATII DNA. An increase in copy number as compared to a normal cell, and/or an increase in levels of HSATII RNA, indicates that the cancer is susceptible to a treatment described herein. Thus, the methods can include detecting and/or identifying a cancer that has high levels of HSATII RNA and/or an increased HSATII copy number, and/or selecting a subject who has a cancer with high levels of HSATII RNA and/or an increased HSATII copy number, for treatment with a method described herein.

In some embodiments, the methods include determining TP53 status of the cancer, and selecting a cancer that harbors a mutation in a TP53 allele (or not selecting a cancer that has wild type TP53). Reference genomic sequences for TP53 can be found at NG_017013.2 (Range 5001-24149, RefSeqGene); NC_000017.11 (Range 7668402-7687550, Reference GRCh38.p2 Primary Assembly). The methods can include obtaining a sample containing cells from a subject, and evaluating the presence of a mutation in TP53 as known in the art or described herein in the sample, e.g., by comparing the sequence of TP53 in the sample to a reference sequence, e.g., a reference that represents a sequence in a normal (wild-type) or non-cancerous cell, or a disease reference that represents a sequence in a cell from a cancer, e.g., a malignant cell. A mutation in TP53 associated with susceptibility to treatment using a method described herein is a sequence that is different from the reference sequence (e.g., as provided herein) at one or more positions. In some embodiments, the mutation is a mutation known in the art to be associated with cancer. The International Agency for Research on Cancer maintains a database of TP53 mutations found in human cancers, available online at p53.iarc.fr (version R18, April 2016); see also Petitjean et al. (2007) Hum. Mutat. 28(6): 622-9 and Bouaoun et al. (2016) Hum. Mutat. 37(9): 865-76. In some embodiments, the mutation is a mutation at codon 175, 245, 248, 249, 273, or 282. See, e.g., Olivier et al. (2010) Cold Spring Harb. Perspect. Biol. 2(1): a001008.

The presence of a mutation in a TP53, and/or HSATII RNA levels and/or HSATII copy number, can be evaluated using methods known in the art, e.g., using polymerase chain reaction (PCR), reverse transcriptase polymerase chain reaction (RT-PCR), quantitative or semi-quantitative real-time RT-PCR, digital PCR, i.e., BEAMing (Beads, Emulsion, Amplification, Magnetics), Diehl (2006) Nat Methods 3: 551-559); RNAse protection assay; Northern blot; various types of nucleic acid sequencing (Sanger, pyrosequencing, NextGeneration Sequencing); fluorescent in-situ hybridization (FISH); or gene array/chips); RNA in situ hybridization (RNA ISH); RNA expression assays, e.g., microarray analysis; multiplexed gene expression analysis methods, e.g., RT-PCR, RNA-sequencing, and oligo hybridization assays including RNA expression microarrays; hybridization based digital barcode quantification assays such as the nCounter® System (NanoString Technologies, Inc., Seattle, Wash.; Kulkarni (2011) Curr. Protoc. Mol. Biol. Chapter 25: Unit25B.10), and lysate based hybridization assays utilizing branched DNA signal amplification such as the QuantiGene 2.0 Single Plex and Multiplex Assays (Affymetrix, Inc., Santa Clara, Calif.; see, e.g., Linton et al. (2012) J. Mol. Diagn. 14(3): 223-32); SAGE, high-throughput sequencing, multiplex PCR, MLPA, Luminex/XMAP, or branched DNA analysis methods. See, e.g., International Publication No. WO 2012/048113, which is incorporated herein by reference in its entirety.

In some embodiments, the methods include determining LINE-1 status of the cancer, and selecting a cancer that harbors a mutation in a LINE-1 allele (or not selecting a cancer that has wild type LINE-1). See, e.g., International Publication No. WO 2012/048113, which is incorporated herein by reference in its entirety. The methods can include obtaining a sample containing cells from a subject, and evaluating the presence of a mutation in LINE-1 as known in the art or described herein in the sample, e.g., by comparing the sequence of LINE-1 in the sample to a reference sequence, e.g., a reference that represents a sequence in a normal (wild-type) or non-cancerous cell, or a disease reference that represents a sequence in a cell from a cancer, e.g., a malignant cell. A mutation in LINE-1 associated with susceptibility to treatment using a method described herein is a sequence that is different from the reference sequence (e.g., as provided herein) at one or more positions.

In some embodiments, RNA ISH is used. Certain RNA ISH platforms leverage the ability to amplify the signal within the assay via a branched-chain technique of multiple polynucleotides hybridized to one another (e.g., bDNA) to form a branch structure (e.g., branched nucleic acid signal amplification). In addition to its high sensitivity, the platform also has minimal non-specific background signal compared to immunohistochemistry (see e.g., Urbanek et al. (2015) Int. J Mol. Sci. 16(6): 13259-86).

In some embodiments, the assay is a bDNA assay as described in U.S. Pat. Nos. 7,709,198, 7,803,541, and 8,114,681; and U.S. Publication No. 2006/0263769, which describe the general bDNA approach; see especially 14:39 through 15:19 of the '198 patent. In some embodiments, the methods include using a modified RNA in situ hybridization (ISH) technique using a branched-chain DNA assay to directly detect and evaluate the level of biomarker mRNA in the sample (see, e.g., U.S. Pat. No. 7,803,541B2; Canales et al. (2006) Nat. Biotechnol. 24(9):1115-22; Ting et al. (2011) Science 331(6017): 593-6). A kit for performing this assay is commercially-available from Affymetrix, Inc. (e.g., the QuantiGene® ViewRNA Assays for tissue and cell samples).

RNA ISH can be performed, e.g., using the QuantiGene® ViewRNA technology (Affymetrix, Santa Clara, Calif.). QuantiGene® ViewRNA ISH is based on the branched DNA technology wherein signal amplification is achieved via a series of sequential steps (e.g., in a single plex format or a two plex format). Thus, in some embodiments, the methods include performing an assay as described in US Publication No. 2012/0052498 (which describes methods for detecting both a nucleic acid and a protein with bDNA signal amplification, comprising providing a sample comprising or suspected of comprising a target nucleic acid and a target protein; incubating at least two label extender probes each comprising a different L-1 sequence, an antibody specific for the target protein, and at least two label probe systems with the sample comprising or suspected of comprising the target nucleic acid and the target protein, wherein the antibody comprises a pre-amplifier probe, and wherein the at least two label probe systems each comprise a detectably different label; and detecting the detectably different labels in the sample); US Publication No. 2012/0004132; US Publication No. 2012/0003648 (which describes methods of amplifying a nucleic acid detection signal comprising hybridizing one or more label extender probes to a target nucleic acid; hybridizing a pre-amplifier to the one or more label extender probes; hybridizing one or more amplifiers to the pre-amplifier; hybridizing one or more label spoke probes to the one or more amplifiers; and hybridizing one or more label probes to the one or more label spoke probes); or US Publication No. 2012/0172246 (which describes methods of detecting a target nucleic acid sequence, comprising providing a sample comprising or suspected of comprising a target nucleic acid sequence; incubating at least two label extender probes each comprising a different L-1 sequence, and a label probe system with the sample comprising or suspected of comprising the target nucleic acid sequence; and detecting whether the label probe system is associated with the sample). Each hybridized target specific polynucleotide probe acts in turn as a hybridization target for a pre-amplifier polynucleotide that in turn hybridizes with one or more amplifier polynucleotides. In some embodiments two or more target specific probes (label extenders) are hybridized to the target before the appropriate pre-amplifier polynucleotide is bound to the 2 label extenders, but in other embodiments a single label extender can also be used with a pre-amplifier. Thus, in some embodiments the methods include incubating one or more label extender probes with the sample. In some embodiments, the target specific probes (label extenders) are in a ZZ orientation, cruciform orientation, or other (e.g., mixed) orientation; see, e.g., FIGS. 10A and 10B of US Publication No. 2012/0052498. Each amplifier molecule provides binding sites to multiple detectable label probe oligonucleotides, e.g., chromogen or fluorophore conjugated-polynucleotides, thereby creating a fully assembled signal amplification “tree” that has numerous binding sites for the label probe; the number of binding sites can vary depending on the tree structure and the labeling approach being used, e.g., from 16-64 binding sites up to 3000-4000 range. In some embodiments there are 300-5000 probe binding sites. The number of binding sites can be optimized to be large enough to provide a strong signal but small enough to avoid issues associated with overlarge structures, i.e., small enough to avoid steric effects and to fairly easily enter the fixed/permeabilized cells and be washed out of them if the target is not present, as larger trees will require larger components that may get stuck within pores of the cells (e.g., the pores created during permeabilization, the pores of the nucleus) despite subsequent washing steps and lead to noise generation.

In some embodiments, the label probe polynucleotides are conjugated to an enzyme capable of interacting with a suitable chromogen, e.g., alkaline phosphatase (AP) or horseradish peroxidase (HRP). Where an alkaline phosphatase (AP)-conjugated polynucleotide probe is used, following sequential addition of an appropriate substrate such as fast red or fast blue substrate, AP breaks down the substrate to form a precipitate that allows in-situ detection of the specific target RNA molecule. Alkaline phosphatase can be used with a number of substrates, e.g., fast red, fast blue, or 5-Bromo-4-chloro-3-indolyl-phosphate (BCIP). Thus, in some embodiments, the methods include the use of alkaline phosphatase conjugated polynucleotide probes within a bDNA signal amplification approach, e.g., as described generally in U.S. Pat. Nos. 5,780,277 and 7,033,758. Other enzyme and chromogenic substrate pairs can also be used, e.g., horseradish peroxidase (HRP) and 3,3′-Diaminobenzidine (DAB). Many suitable enzymes and chromogen substrates are known in the art and can be used to provide a variety of colors for the detectable signals generated in the assay, and suitable selection of the enzyme(s) and substrates used can facilitate multiplexing of targets and labels within a single sample. For these embodiments, labeled probes can be detected using known imaging methods, e.g., bright-field microscopy with a CISH approach.

Other embodiments include the use of fluorophore-conjugates probes, e.g., Alexa Fluor dyes (Life Technologies Corporation, Carlsbad, Calif.) conjugated to label probes. In these embodiments, labeled probes can be detected using known imaging methods, e.g., fluorescence microscopy (e.g., FISH). Selection of appropriate fluorophores can also facilitate multiplexing of targets and labels based upon, e.g., the emission spectra of the selected fluorophores.

In some embodiments, the assay is similar to those described in US Publication Nos. 2012/0100540, 2013/0023433, 2013/0171621, 2012/0071343; or 2012/0214152 (the entire contents of each of the foregoing are incorporated herein by reference in their entirety).

In some embodiments, an RNA ISH assay is performed without the use of bDNA, and the HSATII or TP53 specific probes are directly or indirectly (e.g., via an antibody) labeled with one or more labels as discussed herein.

The assay can be conducted manually or on an automated instrument, such the Leica BOND family of instruments, or the Ventana DISCOVERY ULTRA or DISCOVERY XT instruments.

As used herein, a “test sample” refers to a biological sample obtained from a subject of interest including a cell or cells, e.g., tissue, from a tumor. (Lehninger Biochemistry (Worth Publishers, Inc., current edition); Sambrook et al., Molecular Cloning: A Laboratory Manual, 3d. ed., 2001, Cold Spring Harbor Laboratory Press, New York; Bernard (2002) Clin. Chem. 48(8): 1178-85; Miranda (2010) Kidney International 78: 191-9; Bianchi (2011) EMBO Mol Med 3: 495-503; Taylor (2013) Front. Genet. 4: 142; Yang (2014) PLoS One 9(11): e110641); Nordstrom (2000) Biotechnol. Appl. Biochem. 31(2): 107-12; Ahmadian (2000) Anal. Biochem. 280: 103-10. In some embodiments, high throughput methods, e.g., protein or gene chips as are known in the art (see, e.g., Ch. 12, Genomics, in Griffiths et al., Eds. Modern Genetic Analysis, 1999, W. H. Freeman and Company; Ekins and Chu (1999) Trends in Biotechnology 17: 217-8; MacBeath and Schreiber (2000) Science 289(5485): 1760-3; Simpson, Proteins and Proteomics: A Laboratory Manual, Cold Spring Harbor Laboratory Press; 2002; Hardiman, Microarrays Methods and Applications: Nuts & Bolts, DNA Press, 2003), can be used to detect the presence and/or level of a mutation in TP53.

In some embodiments a technique suitable for the detection of alterations in the structure or sequence of nucleic acids, such as the presence of deletions, amplifications, or substitutions, can be used for the detection of alterations in HSATII or TP53.

In some embodiments, RT-PCR can be used to detect mutations and copy number variants (CNV). The first step in expression profiling by RT-PCR is the reverse transcription of the RNA template into cDNA, followed by its exponential amplification in a PCR reaction (Ausubel et al. (1997) Current Protocols of Molecular Biology, John Wiley and Sons). To minimize errors and the effects of sample-to-sample variation, RT-PCR is usually performed using an internal standard, which is expressed at constant level among tissues, and is unaffected by the experimental treatment. Housekeeping genes as known in the art are most commonly used.

In some embodiments, the methods can include detecting protein levels of TP53, and comparing the protein levels to reference protein levels in a normal cell. A mutation in TP53 typically results in a decrease in protein expression levels, so a decrease in protein expression levels as compared to a wild type reference or threshold level can be used as a proxy for mutation status; a cancer in which tp53 levels are decreased can be selected for treatment with a method described herein (or a cancer in which TP53 levels are normal or not substantially decreased as compared to a wild type reference or threshold can be excluded from treatment with a method described herein). The level of a protein can be evaluated using methods known in the art, e.g., using standard electrophoretic and quantitative immunoassay methods for proteins, including but not limited to, Western blot; enzyme linked immunosorbent assay (ELISA); biotin/avidin type assays; protein array detection; radio-immunoassay; immunohistochemistry (IHC); immune-precipitation assay; FACS (fluorescent activated cell sorting); mass spectrometry (Kim (2010) Am. J. Clin. Pathol. 134: 157-62; Yasun (2012) Anal. Chem. 84(14): 6008-15; Brody (2010) Expert Rev. Mol. Diagn. 10(8): 1013-22; Philips (2014) PLOS One 9(3): e90226; Pfaffe (2011) Clin. Chem. 57(5): 675-687). The methods typically include detectable labels such as fluorescent, chemiluminescent, radioactive, and enzymatic or dye molecules that provide a signal either directly or indirectly. As used herein, the term “label” refers to the coupling (i.e. physically linkage) of a detectable substance, such as a radioactive agent or fluorophore (e.g. phycoerythrin (PE) or indocyanine (Cy5), to an antibody or probe, as well as indirect labeling of the probe or antibody (e.g. horseradish peroxidase, HRP) by reactivity with a detectable substance.

In some embodiments, an enzyme-linked immunosorbent assay (ELISA) method may be used, wherein the wells of a mictrotiter plate are coated with an antibody against which the protein is to be tested. The sample containing or suspected of containing the biological marker is then applied to the wells. After a sufficient amount of time, during which antibody-antigen complexes would have formed, the plate is washed to remove any unbound moieties, and a detectably labelled molecule is added. Again, after a sufficient period of incubation, the plate is washed to remove any excess, unbound molecules, and the presence of the labeled molecule is determined using methods known in the art. Variations of the ELISA method, such as the competitive ELISA or competition assay, and sandwich ELISA, may also be used, as these are well-known to those skilled in the art.

In some embodiments, an immunohistochemistry (IHC) method may be used. IHC provides a method of detecting a biological marker in situ. The presence and exact cellular location of the biological marker can be detected. Typically a sample (e.g., a biopsy sample) is fixed with formalin or paraformaldehyde, embedded in paraffin, and cut into sections for staining and subsequent inspection by light microscopy. Current methods of IHC typically use either direct or indirect labelling. The sample may also be inspected by fluorescent microscopy when immunofluorescence (IF) is performed, as a variation to IHC.

Mass spectrometry, and particularly matrix-assisted laser desorption/ionization mass spectrometry (MALDI-MS) and surface-enhanced laser desorption/ionization mass spectrometry (SELDI-MS), is useful for the detection of biomarkers of this invention. (See U.S. Pat. Nos. 5,118,937; 5,045,694; 5,719,060; and 6,225,047).

The sample can be, e.g., a biopsy, e.g., needle biopsy or a resection specimen, taken from a mass known or suspected to be a tumor or cancerous.

The reference or predetermined level can be a single cut-off (threshold) value, such as a median or mean, or a level that defines the boundaries of an upper or lower quartile, tertile, or other segment of a cohort, e.g., a clinical trial population, that is determined to be statistically different from the other segments. It can be a range of cut-off (or threshold) values, such as a confidence interval. It can be established based upon comparative groups, such as where association with risk of developing disease or presence of disease in one defined group is a fold higher, or lower, (e.g., approximately 2-fold, 4-fold, 8-fold, 16-fold or more) than the risk or presence of disease in another defined group. It can be a range, for example, where a population of subjects (e.g., control subjects) is divided equally (or unequally) into groups, such as a low-risk group, a medium-risk group and a high-risk group, or into quartiles, the lowest quartile being subjects with the lowest risk and the highest quartile being subjects with the highest risk, or into n-quantiles (i.e., n regularly spaced intervals) the lowest of the n-quantiles being subjects with the lowest risk and the highest of the n-quantiles being subjects with the highest risk.

Subjects associated with predetermined values are typically referred to as reference subjects. For example, in some embodiments, a control reference subject does not have does not have cancer.

In some embodiments, the amount by which the level in the subject is greater than the reference level is sufficient to distinguish a subject from a control subject, and optionally is statistically significantly greater than the level in a control subject. In cases where the copy number in a subject is “equal to” the reference copy number, the “being equal” refers to being approximately equal (e.g., not statistically different).

The predetermined value can depend upon the particular population of subjects (e.g., human subjects) selected. Appropriate ranges and categories can be selected with no more than routine experimentation by those of ordinary skill in the art.

In characterizing likelihood, or risk, numerous predetermined values can be established.

Selection of an appropriate route of administration of the HERV-K blocking agent will depend on various factors including, but not limited to, the particular disorder and/or severity of the disorder. In some embodiments, the HERV-K blocking agent is administered orally, parenterally, intravenously, topically, intraperitoneally, subcutaneously, intracranially, intrathecally, or by inhalation. In some embodiments, the HERV-K blocking agent is administered by continuous infusion.

HER V-K Reverse Transcriptase Blocking Agents

HERV-K RT

Human endogenous retroviruses (HERVs) are retrovirus-like sequences integrated into the human genome (Douville and Nath (2014) Handb. Clin. Neurol. 123: 465-85). There are multiple families of HERVs integrated into the human genome, including gammaretroviruses (HERV-W, HERV-H, HERV-F, HERV-I, and HERV-E), the betaretroviruses (HERV-K, classified as HML 1 to 10), and the spumaretroviruses (HERV-S and HERV-L) (see Blikstad et al. (2008) Cell Mol Life Sci. 65: 3348-65). The HERV-K HML-2 family exists in the genome in proviral form and includes approximately 3000 proviral fragments and at least 91 full length viral elements (see Paces et al. (2004) Nucleic Acids Res. 32: D50, and Subramanian et at. (2011) Retrovirology 8: 90). Each complete HERV-K proviral element consists of three retroviral genes, gag, pol, and env, and two accessory genes (rec and np9) flanked by two long terminal repeats (LTRs) (Hughes and Coffin (2004) Proc. Natl. Acad. Sci. USA 101: 1668-72). The gag gene encodes the gag polyprotein which is cleaved by protease to produce viral matrix, capsid, and nucleocapsid proteins. The pol gene encodes a reverse transcriptase (RT) and an integrase, which are the products of a reading frame-shift during translation (Douville and Nath (2014)).

Several HERV-K retrovirus-like sequences have been sequenced and annotated (see, e.g., HERV-K111, GenBank Accession No. HU476554.2). Although multiple of HERV-K variants have been identified, the full length nucleic acid sequence for an exemplary HERV-K pol gene (GenBank Reference No. Y.18890.1 (Pol gene is nucleotides 4266-6374) is provided below:

>Y18890.1: 4266-6374 Human endogenous retrovirus
type K (HERV-K), gag, pol and env genes
(SEQ ID NO: 1)
ATGATCCCAAAAGATTGGCCTTTAATTATAATTGATCTAAAGGACTGCTT
TTTTACCATCCCTCTGGCAGAGCAGGATTGTGAAAAATTTGCCTTTACTA
TACCAGCCATAAATAATAAAGAACCAGCCACCAGGTTTCAGTGGAAAGTG
TTACCTCAGGGAATGCTTAATAGTCCAACTCTTTGTCAGACTTTTGTAGG
TCGAGCTCTTCAACCAGTTAGAGACAAGTTTTCAGACTGTTATATTATTC
ATTATTTTGATGATATTTTATGTGCTGCAGAAACGAAAGATAAATTAATT
GACTGTTATACATTTCTGCAAGCAGAGGTTGCCAATGCAGGACTGGCAAT
AGCATCTGATAAGATCCAAACCTCTACTCCTTTTCATTATTTAGGGATGC
AGATAGAAAATAGAAAAATTAAGCCACAAAAAATAGAAATAAGAAAAGAC
ACATTAAAAACACTAAATGATTTTCAAAAATTGCTGGGAGATATTAATTG
GATTCGGCCAACTCTAGGCATTCCTACTTATGCCATGTCAAATTTGTTCT
CTATCTTAAGAGGAGACTCAGACTTAAATAGTAAAAGAATGTTAACCCCA
GAGGCAACAAAAGAAATTAAATTAGTGGAAGAAAAAATTCAGTCAGCGCA
AATAAATAGAATAGATCCCTTAGCCCCACTCCAACTTTTGATTTTTGCCA
CTGCCCATTCTCCAACAGGCATCATTATTCAAAATACTGATCTTGTGGAG
TGGTCATTCCTTCCTCACAGTACAGTTAAGACTTTTACATTGTACTTGGA
TCAAATAGCTACTTTAATTGGTCCGACAAGATTACGAATAATAAAATTAT
GTGGAAATGACCCAGACAAAATAGTTGTCCCTTTAACCAAGGAACAAGTT
AGACAAGCCTTTATCAATTCTGGTGCATGGCAGATTGGTCTTGCTAATTT
TGTGGGAATTATTGATAATCATTACCCAAAAACAAAAATCTTCCAGTTCT
TAAAATTGACTACTTGGATTCTACCTAAAATTACCAGACGTGAACCTTTA
GAAAATGCTCTAACAGTATTTACTGATGGTTCCAGCAATGGAAAAGTGGC
TTACACAGGGCCAAAAGAACGAGTAATCAAAACTCCATATCAATCGGCTC
AAAGAGCAGAGTTGGTTGCAGTCATTACAGTGTTACAAGATTTTGATCAA
CCTATCAATATTATATCGGATTCTGCATATGTAGTACAGGCTACAAGGGA
TGTTGAGACAGCTCTAATTAAATATAGCATGGACGATCAGTTAAACCAGC
TATTCAATTTATTACAACAAACTGTAAGAAAAAGAAACTTCCCATTTTAT
ATTACTCATATTCGAGCACACACTAATTTACCAGGGCCTTTGACTAAAGC
AAATGAACAAGCTGACTTACTGGTATCATCTGCATTCATAAAAGCACAAG
AACTTCATGCTTTGACTCATGTAAATGCAGCAGGATTAAAAAACAAATTT
GATGTCACATGGAAACAGGCAAAAGATATTGTACAACATTGCACCCAGTG
TCAAGTCTTAGACCTGCCCACTCAAGAGGCAGGAGTTAACCCAGAGGTCT
GTGTCCTAATGCATTATGGCAAATGGATGTCACACATGTACCTTCATTTG
GGAAGATTATCATATGTTCATGTAACAGTTGATACTTATTCACATTTCAT
GTGTGCAACTTGCCAAACAGGAGAAAGTACTTCCCATGTTAAAAAACATT
TATTGTCTTGTTTTGCTGTAATGGGAGTTCCAGAAAAAATCAAAACTGAC
AATGGACCAGGATATTGTAGTAAAGCTTTCCAAAAATTCTTAAGTCAGTG
GAAAATTTCACATACAACAGGAATTCCTTATAATTCCCAAGGACAGGCCA
TAGTTGAAAGAACTAATAGAACACTCAAAACTCAATTAGTTAAACAAAAA
GAAGGGGGAGACAGTAAGGAGTGTACCACTCCTCAGATGCAACTTAATCT
AGCACTCTATACTTTAAATTTTTTAAACATTTATAGAAATCAGACTACTA
CTTCTGCAGAACATCTTACTGGTAAAAAGAACAGCCCACATGAAGGAAAA
CTAATTTAG

An exemplary HERV-K RT amino acid sequence above is provided below (GenBank Accession No. CAB56603.1; see also Tonjes et al. (1999) J. Virol. 73: 9187-95):

>CAB56603.1 Pol protein - Human endogenous
retrovirus K
(SEQ ID NO: 2)
MIPKDWPLIIIDLKDCFFTIPLAEQDCEKFAFTIPAINNKEPATRFQWKV
LPQGMLNSPTLCQTFVGRALQPVRDKFSDCYIIHYFDDILCAAETKDKLI
DCYTFLQAEVANAGLAIASDKIQTSTPFHYLGMQIENRKIKPQKIEIRKD
TLKTLNDFQKLLGDINWIRPTLGIPTYAMSNLFSILRGDSDLNSKRMLTP
EATKEIKLVEEKIQSAQINRIDPLAPLQLLIFATAHSPTGIIIQNTDLVE
WSFLPHSTVKTFTLYLDQIATLIGPTRLRIIKLCGNDPDKIVVPLTKEQV
RQAFINSGAWQIGLANFVGIIDNHYPKTKIFQFLKLTTWILPKITRREPL
ENALTVFTDGSSNGKVAYTGPKERVIKTPYQSAQRAELVAVITVLQDFDQ
PINTISDSAYVVQATRDVETALIKYSMDDQLNQLFNLLQQTVRKRNFPFY
ITHIRAHTNLPGPLTKANEQADLLVSSAFIKAQELHALTHVNAAGLKNKF
DVTWKQAKDIVQHCTQCQVLDLPTQEAGVNPEVCVLMHYGKWMSHMYLHL
GRLSYVHVTVDTYSHFMCATCQTGESTSHVKKHLLSCFAVMGVPEKIKTD
NGPGYCSKAFQKFLSQWKISHTTGIPYNSQGQAIVERTNRTLKTQLVKQK
EGGDSKECTTPQMQLNLALYTLNFLNIYRNQTTTSAEHLTGKKNSPHEGK
LI

In some embodiments, the methods described herein comprise the use of a HERV-K Reverse Transcriptase (HERV-K RT) blocking agent.

In some embodiments, the HERV-K RT blocking agent inhibits the expression of HERV-K RT (e.g., by inhibiting the expression of the HERV-K RT protein of SEQ ID NO: 2).

Inhibitory Nucleic Acids

In some embodiments, the HERV-K RT blocking agent partially or completely reduces HERV-K RT protein levels. Inhibition of HERV-K RT protein levels can be achieved at the DNA, RNA or protein level. For example, the HERV-K RT blocking agent may comprise one or more agents suitable for use in genome editing techniques that can knockout or disrupt a gene encoding HERV-K RT (e.g., a HERV-K pol gene), or prevent the initiation of transcription of a gene encoding HERV-K RT. Perturbations of an mRNA encoding a HERV-K RT through disruption of gene splicing, mRNA stability, and/or mRNA translation can also result in decreased HERV-K RT protein levels. Finally, at the protein level, HERV-K RT levels can be modulated by targeting the protein for degradation.

In some embodiments, the HERV-K RT blocking agent is an inhibitory nucleic acid. An inhibitory nucleic acid that binds “specifically” binds primarily to the target RNA encoding HERV-K RT to inhibit the target RNA but not non-target RNAs. The specificity of the nucleic acid interaction thus refers to its function (e.g., inhibiting the expression of a gene encoding HERV-K RT) rather than its hybridization capacity. Inhibitory nucleic acids may exhibit nonspecific binding to other sites in the genome or other RNAs, without interfering with binding of other regulatory proteins and without causing degradation of the non-specifically-bound RNA. Thus, this nonspecific binding does not significantly affect function of other non-target RNAs and results in no significant adverse effects.

In some embodiments, the methods described herein include administering a composition, e.g., a sterile composition, comprising an inhibitory nucleic acid that is complementary to nucleic acid comprising a gene encoding a HERV-K RT. Inhibitory nucleic acids for use in practicing the methods described herein can be an antisense or small interfering RNA, including but not limited to a shRNA or siRNA. In some embodiments, the inhibitory nucleic acid is a modified nucleic acid polymer (e.g., a locked nucleic acid (LNA) molecule). Inhibitory nucleic acids have been employed as therapeutic moieties in the treatment of disease states in animals, including humans. Inhibitory nucleic acids can be useful therapeutic modalities that can be configured to be useful in treatment regimens for the treatment of cells, tissues, and animals, especially humans.

Inhibitory nucleic acids useful in the present methods and compositions include antisense oligonucleotides, ribozymes, external guide sequence (EGS) oligonucleotides, siRNA compounds, single- or double-stranded RNA interference (RNAi) compounds such as siRNA compounds, molecules comprising modified bases, locked nucleic acid molecules (LNA molecules), antagomirs, peptide nucleic acid molecules (PNA molecules), and other oligomeric compounds or oligonucleotide mimetics which hybridize to at least a portion of the target nucleic acid and modulate its function. In some embodiments, the inhibitory nucleic acids include antisense RNA, antisense DNA, chimeric antisense oligonucleotides, antisense oligonucleotides comprising modified linkages, interference RNA (RNAi), short interfering RNA (siRNA); a micro, interfering RNA (miRNA); a small, temporal RNA (stRNA); or a short, hairpin RNA (shRNA); small RNA-induced gene activation (RNAa); small activating RNAs (saRNAs), or combinations thereof. See, e.g., International Publication No. WO 2010/040112.

In the present methods, the inhibitory nucleic acids are preferably designed to target a nucleic acid encoding a HERV-K RT.

In some embodiments, the inhibitory nucleic acids are 10 to 50, 13 to 50, or 13 to 30 nucleotides in length. One of ordinary skill in the art will appreciate that this embodies oligonucleotides having antisense (complementary) portions of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length, or any range therewithin. It is understood that non-complementary bases may be included in such inhibitory nucleic acids; for example, an inhibitory nucleic acid 30 nucleotides in length may have a portion of 15 bases that is complementary to the targeted RNA. In some embodiments, the oligonucleotides are 15 nucleotides in length. In some embodiments, the antisense or oligonucleotide compounds of the invention are 12 or 13 to 30 nucleotides in length. One of ordinary skill in the art will appreciate that this embodies inhibitory nucleic acids having antisense (complementary) portions of 12, 13, 14, 15, 16, 17, 18, 19, 20, 21, 22, 23, 24, 25, 26, 27, 28, 29, or 30 nucleotides in length, or any range therewithin.

Preferably, the inhibitory nucleic acid comprises one or more modifications comprising: a modified sugar moiety, and/or a modified internucleoside linkage, and/or a modified nucleotide and/or combinations thereof. It is not necessary for all positions in a given oligonucleotide to be uniformly modified, and in fact more than one of the modifications described herein may be incorporated in a single oligonucleotide or even at within a single nucleoside within an oligonucleotide.

In some embodiments, the inhibitory nucleic acids are chimeric oligonucleotides that contain two or more chemically distinct regions, each made up of at least one nucleotide. These oligonucleotides typically contain at least one region of modified nucleotides that confers one or more beneficial properties (such as, for example, increased nuclease resistance, increased uptake into cells, increased binding affinity for the target) and a region that is a substrate for enzymes capable of cleaving RNA:DNA or RNA:RNA hybrids. Chimeric inhibitory nucleic acids may be formed as composite structures of two or more oligonucleotides, modified oligonucleotides, oligonucleosides, and/or oligonucleotide mimetics as described above. Such compounds have also been referred to in the art as hybrids or gapmers. Representative U.S. patents that teach the preparation of such hybrid structures comprise, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference.

In some embodiments, the inhibitory nucleic acid comprises at least one nucleotide modified at the 2′ position of the sugar, most preferably a 2′-O-alkyl, 2′-O-alkyl-O-alkyl or 2′-fluoro-modified nucleotide. In other preferred embodiments, RNA modifications include 2′-fluoro, 2′-amino and 2′ O-methyl modifications on the ribose of pyrimidines, abasic residues or an inverted base at the 3′ end of the RNA. Such modifications are routinely incorporated into oligonucleotides and these oligonucleotides have been shown to have a higher Tm (i.e., higher target binding affinity) than; 2′-deoxyoligonucleotides against a given target.

A number of nucleotide and nucleoside modifications have been shown to make the oligonucleotide into which they are incorporated more resistant to nuclease digestion than the native oligodeoxynucleotide; these modified oligos survive intact for a longer time than unmodified oligonucleotides. Specific examples of modified oligonucleotides include those comprising modified backbones, for example, phosphorothioates, phosphotriesters, methyl phosphonates, short chain alkyl or cycloalkyl intersugar linkages or short chain heteroatomic or heterocyclic intersugar linkages. Most preferred are oligonucleotides with phosphorothioate backbones and those with heteroatom backbones, particularly CH₂—NH—O—CH₂, CH,˜N(CH₃)˜O˜CH₂ (known as a methylene(methylimino) or MMI backbone, CH₂—O—N(CH₃)—CH₂, CH₂—N(CH₃)—N (CH₃)—CH₂ and O—N(CH₃)—CH₂—CH₂ backbones, wherein the native phosphodiester backbone is represented as O— P—O— CH,); amide backbones (see De Mesmaeker et al. (1995) Ace. Chem. Res. 28: 366-74); morpholino backbone structures (see U.S. Pat. No. 5,034,506); peptide nucleic acid (PNA) backbone (wherein the phosphodiester backbone of the oligonucleotide is replaced with a polyamide backbone, the nucleotides being bound directly or indirectly to the aza nitrogen atoms of the polyamide backbone, see Nielsen et al. (1991) Science 254, 1497-500). Phosphorus-containing linkages include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′ (see U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455, 233; 5,466,677; 5,476,925; 5,519,126; 5,536,821; 5,541,306; 5,550,111; 5,563, 253; 5,571,799; 5,587,361; and 5,625,050; the disclosures of which are incorporated herein by reference in their entireties).

Morpholino-based oligomeric compounds are described in Dwaine et al. (2002) Biochemistry 41(14): 4503-10; Genesis 30(3), 2001; Heasman (2002) Dev. Biol. 243: 209-14; Nasevicius et al. (2000) Nat. Genet. 26: 216-20; Lacerra et al. (2000) Proc. Natl. Acad. Sci. U.S.A. 97: 9591-6; and U.S. Pat. No. 5,034,506. In some embodiments, the morpholino-based oligomeric compound is a phosphorodiamidate morpholino oligomer (PMO) (e.g., as described in Iverson (2001) Curr. Opin. Mol. Ther. 3: 235-8; and Wang et al. (2010) J. Gene Med. 12: 354-64; the disclosures of which are incorporated herein by reference in their entireties).

Cyclohexenyl nucleic acid oligonucleotide mimetics are described in Wang et al. (2000) J. Am. Chem. Soc. 122: 8595-602, the contents of which are incorporated herein by reference

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These comprise those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S, and CH₂ component parts; see U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

Modified oligonucleotides are also known that include oligonucleotides that are based on or constructed from arabinonucleotide or modified arabinonucleotide residues. Arabinonucleosides are stereoisomers of ribonucleosides, differing only in the configuration at the 2′-position of the sugar ring. In some embodiments, a 2′-arabino modification is 2′-F arabino. In some embodiments, the modified oligonucleotide is 2′-fluoro-D-arabinonucleic acid (FANA) (as described in, e.g., Lon et al. (2002) Biochem. 41: 3457-67; and Min et al. (2002) Bioorg. Med. Chem. Lett. 12: 2651-4; the disclosures of which are incorporated herein by reference in their entireties). Similar modifications can also be made at other positions on the sugar, particularly the 3′ position of the sugar on a 3′ terminal nucleoside or in 2′-5′ linked oligonucleotides and the 5′ position of 5′ terminal nucleotide.

International Publication No. WO 99/67378 discloses arabinonucleic acids (ANA) oligomers and their analogues for improved sequence specific inhibition of gene expression via association to complementary messenger RNA.

Other preferred modifications include ethylene-bridged nucleic acids (ENAs) (e.g., International Publication No. WO 2005/042777, Morita et al. (2001) Nucleic Acid Res. Suppl. 1: 241-2; Surono et al. (2004) Hum. Gene Ther. 15: 749-57; Koizumi (2006) Curr. Opin. Mol. Ther. 8: 144-9; and Horie et al. (2005) Nucleic Acids Symp. Ser. (Oxf), 49: 171-2; the disclosures of which are incorporated herein by reference in their entireties). Preferred ENAs include, but are not limited to, 2′-0,4′-C-ethylene-bridged nucleic acids.

Examples of LNAs are described in International Publication No. WO 2008/043753 and include compounds of the following formula.

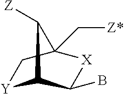

where X and Y are independently selected among the groups —O—, —S—, —N(H)—, N(R)—, —CH₂— or —CH— (if part of a double bond), —CH₂—O—, —CH₂—S—, —CH₂—N(H)—, —CH₂—N(R)—, —CH₂—CH₂— or —CH₂—CH— (if part of a double bond),
—CH═CH—, where R is selected from hydrogen and C₁₋₄-alkyl; Z and Z* are independently selected among an internucleoside linkage, a terminal group or a protecting group; B constitutes a natural or non-natural nucleotide base moiety; and the asymmetric groups may be found in either orientation.

Preferably, the LNA used in the oligomer of the invention comprises at least one LNA unit according any of the formulas

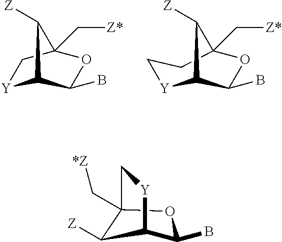

wherein Y is —O—, —S—, —NH—, or N(R^{H}); Z and Z* are independently selected among an internucleoside linkage, a terminal group or a protecting group; B constitutes a natural or non-natural nucleotide base moiety, and RH is selected from hydrogen and C₁₋₄-alkyl.

Preferably, the LNA used in the oligomeric compound, such as an antisense oligonucleotide, of the invention comprises at least one nucleotide comprises a LNA unit according any of the formulas shown in “Scheme 2” of PCT/DK2006/000512.

Preferably, the LNA used in the oligomer of the invention comprises internucleoside linkages selected from —O—P(O)₂—O—, —O—P(O,S)—O—, —O—P(S)₂—O—, —S—P(O)₂—O—, —S—P(O,S)—O—, —S—P(S)₂—O—, —O—P(O)₂—S—, —O—P(O,S)—S—, —S—P(O)₂—S—, —O—PO(R^{H})—O—O—PO(OCH₃)—O—, —O—PO(NR^{H})—O—, —O—PO(OCH₂CH₂S—R)—O—, —O—PO(BH₃)—O—, —O—PO(NHR^{H})—O—, —O—P(O)₂—NR^{H}—, —NR^{H}—P(O)₂—O—, —NR^{H}—CO—O—, where R^{H} is selected from hydrogen and C₁₋₄-alkyl.

Specifically, preferred LNA units are shown in Scheme 1:

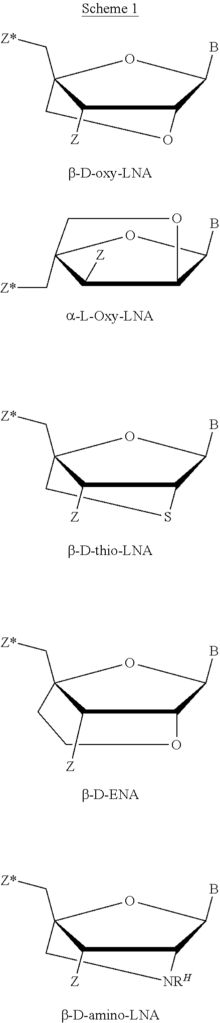

The term “thio-LNA” comprises a locked nucleotide in which at least one of X or Y in the general formula above is selected from S or —CH₂—S—. Thio-LNA can be in both beta-D and alpha-L-configuration.

The term “amino-LNA” comprises a locked nucleotide in which at least one of X or Y in the general formula above is selected from —N(H)—, N(R)—, CH₂—N(H)—, and —CH₂— N(R)— where R is selected from hydrogen and C₁₋₄-alkyl. Amino-LNA can be in both beta-D and alpha-L-configuration.

The term “oxy-LNA” comprises a locked nucleotide in which at least one of X or Y in the general formula above represents —O— or —CH₂—O—. Oxy-LNA can be in both beta-D and alpha-L-configuration.

The term “ena-LNA” comprises a locked nucleotide in which Y in the general formula above is —CH₂—O— (where the oxygen atom of —CH₂—O— is attached to the 2′-position relative to the base B). LNAs are described in additional detail below.

One or more substituted sugar moieties can also be included, e.g., one of the following at the 2′ position: OH, SH, SCH₃, F, OCN, OCH₃, OCH₃ O(CH₂)n CH₃, O(CH₂)n NH₂ or O(CH₂)n CH₃ where n is from 1 to about 10; C₁ to C₁₀ lower alkyl, alkoxyalkoxy, substituted lower alkyl, alkaryl or aralkyl; Cl; Br; CN; CF₃; OCF₃; O—, S—, or N-alkyl; O—, S—, or N-alkenyl; SOCH₃; SO₂ CH₃; ONO₂; NO₂; N₃; NH₂; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. A preferred modification includes 2′-methoxyethoxy [2′-O—CH₂CH₂OCH₃, also known as 2′-O—(2-methoxyethyl)] (Martin et al, Helv. Chim. Acta, 1995, 78, 486). Other preferred modifications include 2′-methoxy (2′-O—CH₃), 2′-propoxy (2′—OCH₂ CH₂CH₃) and 2′-fluoro (2′-F). Similar modifications may also be made at other positions on the oligonucleotide, particularly the 3′ position of the sugar on the 3′ terminal nucleotide and the 5′ position of 5′ terminal nucleotide. Oligonucleotides may also have sugar mimetics such as cyclobutyls in place of the pentofuranosyl group.

Inhibitory nucleic acids can also include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, isocytosine, pseudoisocytosine, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 5-propynyluracil, 8-azaguanine, 7-deazaguanine, N6 (6-aminohexyl)adenine, 6-aminopurine, 2-aminopurine, 2-chloro-6-aminopurine and 2,6-diaminopurine or other diaminopurines. See, e.g., Kornberg (1980) DNA Replication, W. H. Freeman & Co., San Francisco, pp. 75-77; and Gebeyehu et al. (1987) Nucl. Acids Res. 15: 4513-34). A “universal” base known in the art, e.g., inosine, can also be included. 5-Me-C substitutions have been shown to increase nucleic acid duplex stability by 0.6-1.2<0>C. (Sanghvi, in Crooke, and Lebleu, eds., Antisense Research and Applications, CRC Press, Boca Raton, 1993, pp. 276-278).

It is not necessary for all positions in a given oligonucleotide to be uniformly modified, and in fact more than one of the modifications described herein may be incorporated in a single oligonucleotide or even at within a single nucleoside within an oligonucleotide.

In some embodiments, both a sugar and an internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, for example, an aminoethylglycine backbone. The nucleobases are retained and are bound directly or indirectly to aza nitrogen atoms of the amide portion of the backbone. Representative U.S. patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al. (1991) Science 254: 1497-1500.

Inhibitory nucleic acids can also include one or more nucleobase (often referred to in the art simply as “base”) modifications or substitutions. As used herein, “unmodified” or “natural” nucleobases comprise the purine bases adenine (A) and guanine (G), and the pyrimidine bases thymine (T), cytosine (C) and uracil (U). Modified nucleobases comprise other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudo-uracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylquanine and 7-methyladenine, 8-azaguanine and 8-azaadenine, 7-deazaguanine and 7-deazaadenine and 3-deazaguanine and 3-deazaadenine.

Further, nucleobases comprise those disclosed in U.S. Pat. No. 3,687,808, those disclosed in “The Concise Encyclopedia of Polymer Science And Engineering”, pages 858-9, Kroschwitz, ed. John Wiley & Sons, 1990; those disclosed by Englisch et al. (1991) Angewandle Chemie, International Edition 30: 613; and those disclosed by Sanghvi et al. Antisense Research and Applications, chapter 15, pp. 289-302, Crooke and Lebleu, eds., CRC Press, 1993. Certain of these nucleobases are particularly useful for increasing the binding affinity of the oligomeric compounds. These include 5-substituted pyrimidines, 6-azapyrimidines and N-2, N-6 and 0-6 substituted purines, comprising 2-aminopropyladenine, 5-propynyluracil and 5-propynylcytosine. 5-methylcytosine substitutions have been shown to increase nucleic acid duplex stability by 0.6-1.2° C. (Sanghvi et al., Antisense Research and Applications, pp. 276-8, Crooke and Lebleu, eds., CRC Press, 1993) and are presently preferred base substitutions, even more particularly when combined with 2′-O-methoxyethyl sugar modifications. Modified nucleobases are described in U.S. Pat. Nos. 3,687,808; 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,596,091; 5,614,617; 5,750,692; and 5,681,941, each of which is herein incorporated by reference.

In some embodiments, the inhibitory nucleic acids are chemically linked to one or more moieties or conjugates that enhance the activity, cellular distribution, or cellular uptake of the oligonucleotide. For example, one or more inhibitory nucleic acids, of the same or different types, can be conjugated to each other; or inhibitory nucleic acids can be conjugated to targeting moieties with enhanced specificity for a cell type or tissue type. Such moieties include, but are not limited to, lipid moieties such as a cholesterol moiety (Letsinger et al. (1989) Proc. Natl. Acad. Sci. U.S.A. 86: 6553-6), cholic acid (Manoharan et al. (1994) Bioorg. Med. Chem. Let. 4: 1053-60), a thioether, e.g., hexyl-S-tritylthiol (Manoharan et al. (1992) Ann. N. Y. Acad. Sci. 660: 306-9; Manoharan et al. (1993) Bioorg. Med. Chem. Let. 3: 2765-70), a thiocholesterol (Oberhauser et al. (1992) Nucl. Acids Res. 20: 533-8), an aliphatic chain, e.g., dodecandiol or undecyl residues (Kabanov et al. (1990) FEBS Lett. 259: 327-30; Svinarchuk et al. (1993) Biochimie 75: 49-54), a phospholipid, e.g., di-hexadecyl-rac-glycerol or triethylammonium 1,2-di-O-hexadecyl-rac-glycero-3-H-phosphonate (Manoharan et al. (1995) Tetrahedron Lett. 36: 3651-4; Shea et al. (1990) Nucl. Acids Res. 18: 3777-83), a polyamine or a polyethylene glycol chain (Mancharan et al. (1995) Nucleosides & Nucleotides 14: 969-973), or adamantane acetic acid (Manoharan et al. (1995) Tetrahedron Lett. 36: 3651-4), a palmityl moiety (Mishra et al. (1995) Biochim. Biophys. Acta 1264: 229-37), or an octadecylamine or hexylamino-carbonyl-t oxycholesterol moiety (Crooke et al. (1996) J. Pharmacol. Exp. Ther. 277: 923-37). See also U.S. Pat. Nos. 4,828,979; 4,948,882; 5,218,105; 5,525,465; 5,541,313; 5,545,730; 5,552, 538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5, 245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5, 565,552; 5,567,810; 5,574,142; 5,585,481; 5,587,371; 5,595,726; 5,597,696; 5,599,923; 5,599,928 and 5,688,941, each of which is herein incorporated by reference.

These moieties or conjugates can include conjugate groups covalently bound to functional groups such as primary or secondary hydroxyl groups. Conjugate groups of the invention include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugate groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve uptake, enhance resistance to degradation, and/or strengthen sequence-specific hybridization with the target nucleic acid. Groups that enhance the pharmacokinetic properties, in the context of this invention, include groups that improve uptake, distribution, metabolism or excretion of the compounds of the present invention. Representative conjugate groups are disclosed in International Publication No. WO 1993/007883, and U.S. Pat. No. 6,287,860, both of which are incorporated herein by reference. Conjugate moieties include, but are not limited to, lipid moieties such as a cholesterol moiety, cholic acid, a thioether, e.g., hexyl-5-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxy cholesterol moiety. See, e.g., U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928; and 5,688,941.

The inhibitory nucleic acids useful in the methods described herein are sufficiently complementary to the target RNA, e.g., hybridize sufficiently well and with sufficient biological functional specificity, to give the desired effect. “Complementary” refers to the capacity for pairing, through base stacking and specific hydrogen bonding, between two sequences comprising naturally or non-naturally occurring (e.g., modified as described above) bases (nucleosides) or analogs thereof. For example, if a base at one position of an inhibitory nucleic acid is capable of hydrogen bonding with a base at the corresponding position of an RNA, then the bases are considered to be complementary to each other at that position. In some embodiments, 100% complementarity is not required. As noted above, inhibitory nucleic acids can comprise universal bases, or inert abasic spacers that provide no positive or negative contribution to hydrogen bonding. Base pairings may include both canonical Watson-Crick base pairing and non-Watson-Crick base pairing (e.g., Wobble base pairing and Hoogsteen base pairing). It is understood that for complementary base pairings, adenosine-type bases (A) are complementary to thymidine-type bases (T) or uracil-type bases (U), that cytosine-type bases (C) are complementary to guanosine-type bases (G), and that universal bases such as such as 3-nitropyrrole or 5-nitroindole can hybridize to and are considered complementary to any A, C, U, or T (see Nichols et al. (1994) Nature 369: 492-3 and Loakes et al. (1994) Nucleic Acids Res. 22: 4039-43. Inosine (I) has also been considered in the art to be a universal base and is considered complementary to any A, C, U, or T. See Watkins and Santa Lucia (2005) Nucl. Acids Res. 33(19): 6258-67.

Additional target segments in a nucleic acid are readily identifiable by one having ordinary skill in the art in view of this disclosure. Target segments 5-500 nucleotides in length comprising a stretch of at least five (5) consecutive nucleotides within the protein binding region, or immediately adjacent thereto, are considered to be suitable for targeting as well. Target segments can include sequences that comprise at least the 5 consecutive nucleotides from the 5′-terminus of one of the protein binding regions (the remaining nucleotides being a consecutive stretch of the same RNA beginning immediately upstream of the 5′-terminus of the binding segment and continuing until the inhibitory nucleic acid contains about 5 to about 100 nucleotides). Similarly preferred target segments are represented by RNA sequences that comprise at least the 5 consecutive nucleotides from the 3-terminus of one of the illustrative preferred target segments (the remaining nucleotides being a consecutive stretch of the same RNA beginning immediately downstream of the 3′-terminus of the target segment and continuing until the inhibitory nucleic acid contains about 5 to about 100 nucleotides). One having skill in the art armed with the sequences provided herein will be able, without undue experimentation, to identify further preferred protein binding regions to target with complementary inhibitory nucleic acids.

It is understood in the art that a complementary nucleic acid sequence need not be 100% complementary to that of its target nucleic acid to be specifically hybridizable. A complementary nucleic acid sequence for purposes of the present methods is specifically hybridizable when binding of the sequence to the target RNA molecule interferes with the normal function of the target RNA to cause a loss of activity (e.g., inhibiting the translation of an mRNA encoding a HERV-K RT) and there is a sufficient degree of complementarity to avoid non-specific binding of the sequence to non-target RNA sequences under conditions in which avoidance of the non-specific binding is desired, e.g., under physiological conditions in the case of in vivo assays or therapeutic treatment, and in the case of in vitro assays, under conditions in which the assays are performed under suitable conditions of stringency. For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (1977) Science 196: 180); Grunstein and Hogness (1975) Proc. Natl. Acad. Sci. USA 72: 3961); Ausubel et al. (2001) Current Protocols in Molecular Biology, Wiley Interscience, New York); Berger and Kimmel (1987) Guide to Molecular Cloning Techniques, Academic Press, New York); and Sambrook et al., Molecular Cloning: A Laboratory Manual, 3d. ed., 2001, Cold Spring Harbor Laboratory Press, New York.

In general, the inhibitory nucleic acids useful in the methods described herein have at least 80% sequence complementarity to a target region within the target nucleic acid, e.g., 80%, 85%, 90%, 95%, or 100% sequence complementarity to the target region within an RNA. For example, an antisense compound in which 18 of 20 nucleobases of the antisense oligonucleotide are complementary, and would therefore specifically hybridize, to a target region would represent 90 percent complementarity. Percent complementarity of an inhibitory nucleic acid with a region of a target nucleic acid can be determined routinely using basic local alignment search tools (BLAST programs) (Altschul et al. (1990) J. Mol. Biol. 215: 403-10; Zhang and Madden (1997) Genome Res. 7: 649-56). Antisense and other compounds of the invention that hybridize to an RNA are identified through routine experimentation. In general the inhibitory nucleic acids must retain specificity for their target, i.e., either do not directly bind to, or do not directly significantly affect expression levels of, transcripts other than the intended target.

Target-specific effects, with corresponding target-specific functional biological effects, are possible even when the inhibitory nucleic acid exhibits non-specific binding to a large number of non-target RNAs. For example, short 8 base long inhibitory nucleic acids that are fully complementary to a RNA may have multiple 100% matches to hundreds of sequences in the genome, yet may produce target-specific effects, e.g. downregulation of a gene encoding a HERV-K RT. 8-base inhibitory nucleic acids have been reported to prevent exon skipping with a high degree of specificity and reduced off-target effect. See Singh et al. (2009) RNA Biol. 6(3): 341-350. 8-base inhibitory nucleic acids have been reported to interfere with miRNA activity without significant off-target effects. See Obad et al. (2011) Nature Genetics 43: 371-8.

For further disclosure regarding inhibitory nucleic acids, see U.S. Publication Nos. 2010/0317718 (antisense oligos); 2010/0249052 (double-stranded ribonucleic acid (dsRNA)); 2009/0181914 and 2010/0234451 (LNA molecules); 2007/0191294 (siRNA analogues); 2008/0249039 (modified siRNA molecules); and International Publication No. WO 2010/129746 and WO 2010/040112 (inhibitory nucleic acids).

Antisense Oligonucleotides

In some embodiments, the inhibitory nucleic acids are antisense oligonucleotides. Antisense oligonucleotides are typically designed to block expression of a DNA or RNA target by binding to the target and halting expression at the level of transcription, translation, or splicing. Antisense oligonucleotides of the present invention are complementary nucleic acid sequences designed to hybridize under stringent conditions to an RNA in vitro, and are expected to reduce and/or reduce the level of transcription, translation, or splicing of a nucleic acid encoding a HERV-K RT. Thus, oligonucleotides are chosen that are sufficiently complementary to the target, i.e., that hybridize sufficiently well and with sufficient biological functional specificity, to give the desired effect.

Modified Base, including Locked Nucleic Acids (LNAs)

In some embodiments, the inhibitory nucleic acids used in the methods described herein comprise one or more modified bonds or bases. Modified bases include phosphorothioate, methylphosphonate, peptide nucleic acids, or locked nucleic acids (LNAs). Preferably, the modified nucleotides are part of locked nucleic acid molecules, including [alpha]-L-LNAs. LNAs include ribonucleic acid analogues wherein the ribose ring is “locked” by a methylene bridge between the 2′-oxgygen and the 4′-carbon—i.e., oligonucleotides containing at least one LNA monomer, that is, one 2′-O, 4′-C-methylene-β-D-ribofuranosyl nucleotide. LNA bases form standard Watson-Crick base pairs but the locked configuration increases the rate and stability of the basepairing reaction (Jepsen et al. (2004) Oligonucleotides 14: 130-146). LNAs also have increased affinity to base pair with RNA as compared to DNA. These properties render LNAs especially useful as probes for fluorescence in situ hybridization (FISH) and comparative genomic hybridization, as knockdown tools for miRNAs, and as antisense oligonucleotides to target mRNAs or other RNAs, e.g., RNAs as described herein.

The modified base/LNA molecules can include molecules comprising 10-30, e.g., 12-24, e.g., 12, 13, 14, 15, 16, 17, 18, 19, 20, 21, 22, 23, 24, 25, 26, 27, 28, 29, or 30 nucleotides in each strand, wherein one of the strands is substantially identical, e.g., at least 80% (or more, e.g., 85%, 90%, 95%, or 100%) identical, e.g., having 3, 2, 1, or 0 mismatched nucleotide(s), to a target region in the RNA. The modified base/LNA molecules can be chemically synthesized using methods known in the art.

The modified base/LNA molecules can be designed using any method known in the art; a number of algorithms are known, and are commercially available (e.g., on the internet, for example at exiqon.com). See, e.g., You et al. (2006) Nucl. Acids. Res. 34: e60; McTigue et al. (2004) Biochemistry 43: 5388-405; and Levin et al. (2006) Nucl. Acids. Res. 34: e142. For example, “gene walk” methods, similar to those used to design antisense oligos, can be used to optimize the inhibitory activity of a modified base/LNA molecule; for example, a series of oligonucleotides of 10-30 nucleotides spanning the length of a target RNA can be prepared, followed by testing for activity. Optionally, gaps, e.g., of 5-10 nucleotides or more, can be left between the LNAs to reduce the number of oligonucleotides synthesized and tested. GC content is preferably between about 30-60%. General guidelines for designing modified base/LNA molecules are known in the art; for example, LNA sequences will bind very tightly to other LNA sequences, so it is preferable to avoid significant complementarity within an LNA molecule. Contiguous runs of three or more Gs or Cs, or more than four LNA residues, should be avoided where possible (for example, it may not be possible with very short (e.g., about 9-10 nt) oligonucleotides). In some embodiments, the LNAs are xylo-LNAs.

For additional information regarding LNA molecules see U.S. Pat. Nos. 6,268,490; 6,734,291; 6,770,748; 6,794,499; 7,034,133; 7,053,207; 7,060,809; 7,084,125; and 7,572,582; and U.S. Publication Nos. 2010/0267018; 2010/0261175; and 2010/0035968; Koshkin et al. (1998) Tetrahedron 54: 3607-30; Obika et al. (1998) Tetrahedron Lett. 39: 5401-4; Jepsen et al. (2004) Oligonucleotides 14: 130-46; Kauppinen et al. (2005) Drug Disc. Today 2(3): 287-90; and Ponting et al. (2009) Cell 136(4): 629-41, and references cited therein.

As demonstrated herein and previously (see, e.g., International Publication Nos. WO 2012/065143 and WO 2012/087983, incorporated herein by reference), LNA molecules can be used as a valuable tool to manipulate and aid analysis of RNAs. Advantages offered by an LNA molecule-based system are the relatively low costs, easy delivery, and rapid action. While other inhibitory nucleic acids may exhibit effects after longer periods of time, LNA molecules exhibit effects that are more rapid, e.g., a comparatively early onset of activity, are fully reversible after a recovery period following the synthesis of new RNA, and occur without causing substantial or substantially complete RNA cleavage or degradation. One or more of these design properties may be desired properties of the inhibitory nucleic acids of the invention. Additionally, LNA molecules make possible the systematic targeting of domains within much longer nuclear transcripts. Although a PNA-based system has been described earlier, the effects on Xi were apparent only after 24 hours (Beletskii et al. (2001) Proc. Natl. Acad. Sci. U.S.A. 98: 9215-20). The LNA technology enables high-throughput screens for functional analysis of non-coding RNAs and also provides a novel tool to manipulate chromatin states in vivo for therapeutic applications.

In various related aspects, the methods described herein include using LNA molecules to target RNAs for a number of uses, including as a research tool to probe the function of a specific RNA, e.g., in vitro or in vivo. The methods include selecting one or more desired RNAs, designing one or more LNA molecules that target the RNA, providing the designed LNA molecule, and administering the LNA molecule to a cell or animal. The methods can optionally include selecting a region of the RNA and designing one or more LNA molecules that target that region of the RNA.

From a commercial and clinical perspective, the timepoints between about 1 to 24 hours potentially define a window for epigenetic reprogramming. The advantage of the LNA system is that it works quickly, with a defined half-life, and is therefore reversible upon degradation of LNAs, at the same time that it provides a discrete timeframe during which epigenetic manipulations can be made. By targeting nuclear long RNAs, LNA molecules or similar polymers, e.g., xylo-LNAs, might be utilized to manipulate the chromatin state of cells in culture or in vivo, by transiently eliminating the regulatory RNA and associated proteins long enough to alter the underlying locus for therapeutic purposes. In particular, LNA molecules or similar polymers that specifically bind to, or are complementary to, a nucleic acid encoding a HERV-K RT can inhibit the expression of the protein, in a gene-specific fashion.

Interfering RNA, Including siRNA shRNA

In some embodiments, the inhibitory nucleic acid sequence that is complementary to an RNA can be an interfering RNA, including but not limited to a small interfering RNA (“siRNA”) or a small hairpin RNA (“shRNA”). Methods for constructing interfering RNAs are well known in the art. For example, the interfering RNA can be assembled from two separate oligonucleotides, where one strand is the sense strand and the other is the antisense strand, wherein the antisense and sense strands are self-complementary (i.e., each strand comprises nucleotide sequence that is complementary to nucleotide sequence in the other strand; such as where the antisense strand and sense strand form a duplex or double stranded structure); the antisense strand comprises nucleotide sequence that is complementary to a nucleotide sequence in a target nucleic acid molecule or a portion thereof (i.e., an undesired gene) and the sense strand comprises nucleotide sequence corresponding to the target nucleic acid sequence or a portion thereof. Alternatively, interfering RNA is assembled from a single oligonucleotide, where the self-complementary sense and antisense regions are linked by means of nucleic acid based or non-nucleic acid-based linker(s). The interfering RNA can be a polynucleotide with a duplex, asymmetric duplex, hairpin or asymmetric hairpin secondary structure, having self-complementary sense and antisense regions, wherein the antisense region comprises a nucleotide sequence that is complementary to nucleotide sequence in a separate target nucleic acid molecule or a portion thereof and the sense region having nucleotide sequence corresponding to the target nucleic acid sequence or a portion thereof. The interfering can be a circular single-stranded polynucleotide having two or more loop structures and a stem comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof, and wherein the circular polynucleotide can be processed either in vivo or in vitro to generate an active siRNA molecule capable of mediating RNA interference.

In some embodiments, the interfering RNA coding region encodes a self-complementary RNA molecule having a sense region, an antisense region and a loop region. Such an RNA molecule when expressed desirably forms a “hairpin” structure, and is referred to herein as a “shRNA.” The loop region is generally between about 2 and about 10 nucleotides in length. In some embodiments, the loop region is from about 6 to about 9 nucleotides in length. In some embodiments, the sense region and the antisense region are between about 15 and about 20 nucleotides in length. Following post-transcriptional processing, the small hairpin RNA is converted into a siRNA by a cleavage event mediated by the enzyme Dicer, which is a member of the RNase III family. The siRNA is then capable of inhibiting the expression of a gene with which it shares homology. For details, see Brummelkamp et al. (2002) Science 296: 550-3; Lee et al. (2002) Nature Biotechnol. 20: 500-5; Miyagishi and Taira (2002) Nature Biotechnol. 20: 497-500; Paddison et al. (2002) Genes & Dev. 16: 948-58; Paul (2002) Nature Biotechnol. 20: 505-08; Sui (2002) Proc. Natl. Acad. Sci. U.S.A. 99(6): 5515-20; Yu et al. (2002) Proc. Natl. Acad. Sci. U.S.A. 99: 6047-52.

The target RNA cleavage reaction guided by siRNAs is highly sequence specific. In general, siRNA containing a nucleotide sequences identical to a portion of the target nucleic acid are preferred for inhibition. However, 100% sequence identity between the siRNA and the target gene is not required. Thus, the design of the siRNAs has the advantage of being able to tolerate sequence variations that might be expected due to genetic mutation, strain polymorphism, or evolutionary divergence. For example, siRNA sequences with insertions, deletions, and single point mutations relative to the target sequence have also been found to be effective for inhibition. Alternatively, siRNA sequences with nucleotide analog substitutions or insertions can be effective for inhibition. In general the siRNAs must retain specificity for their target, i.e., must not directly bind to, or directly significantly affect expression levels of, transcripts other than the intended target.

Ribozymes

In some embodiments, the inhibitory nucleic acids are ribozymes. Trans-cleaving enzymatic nucleic acid molecules can also be used; they have shown promise as therapeutic agents for human disease (Usman & McSwiggen (1995) Ann. Rep. Med. Chem. 30: 285-94; Christoffersen and Marr (1995) J. Med. Chem. 38: 2023-37). Enzymatic nucleic acid molecules can be designed to cleave specific RNA targets within the background of cellular RNA. Such a cleavage event renders the RNA non-functional.

In general, enzymatic nucleic acids with RNA cleaving activity act by first binding to a target RNA. Such binding occurs through the target binding portion of an enzymatic nucleic acid which is held in close proximity to an enzymatic portion of the molecule that acts to cleave the target RNA. Thus, the enzymatic nucleic acid first recognizes and then binds a target RNA through complementary base pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets.

Several approaches such as in vitro selection (evolution) strategies have been used to evolve new nucleic acid catalysts capable of catalyzing a variety of reactions, such as cleavage and ligation of phosphodiester linkages and amide linkages, (Orgel (1979) Proc. R. Soc. London B 205: 435; Joyce (1989) Gene 82: 83-87; Beaudry et al. (1992) Science 257: 635-41; Joyce (1992) Scientific American 267: 90-97; Breaker et al. (1994) TIBTECH 12: 268; Bartel et al. (1993) Science 261: 1411-8; Szostak (1993) TIBS 17: 89-93; Kumar et al. (1995) FASEB J. 9: 1183; Breaker (1996) Curr. Op. Biotech. 1: 442). The development of ribozymes that are optimal for catalytic activity would contribute significantly to any strategy that employs RNA-cleaving ribozymes for the purpose of regulating gene expression. The hammerhead ribozyme, for example, functions with a catalytic rate (kcat) of about 1 min⁻¹ in the presence of saturating (10 mM) concentrations of Mg²⁺ cofactor. An artificial “RNA ligase” ribozyme has been shown to catalyze the corresponding self-modification reaction with a rate of about 100 min⁻¹. In addition, it is known that certain modified hammerhead ribozymes that have substrate binding arms made of DNA catalyze RNA cleavage with multiple turn-over rates that approach 100 min⁻¹.

Making and Using Inhibitory Nucleic Acids

The nucleic acid sequences used to practice the methods described herein, whether RNA, cDNA, genomic DNA, vectors, viruses or hybrids thereof, can be isolated from a variety of sources, genetically engineered, amplified, and/or expressed/generated recombinantly. If desired, nucleic acid sequences of the invention can be inserted into delivery vectors and expressed from transcription units within the vectors. The recombinant vectors can be DNA plasmids or viral vectors. Generation of the vector construct can be accomplished using any suitable genetic engineering techniques well known in the art, including, without limitation, the standard techniques of PCR, oligonucleotide synthesis, restriction endonuclease digestion, ligation, transformation, plasmid purification, and DNA sequencing, for example as described in Sambrook et al., Molecular Cloning: A Laboratory Manual, 3d. ed., 2001, Cold Spring Harbor Laboratory Press, New York, Coffin et al. (Retroviruses. (1997)) and “RNA Viruses: A Practical Approach” (Alan J. Cann, Ed., Oxford University Press, (2000)).

Preferably, inhibitory nucleic acids of the invention are synthesized chemically. Nucleic acid sequences used to practice this invention can be synthesized in vitro by well-known chemical synthesis techniques, as described in, e.g., Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066; WO/2008/043753 and WO/2008/049085, and the references cited therein.

Nucleic acid sequences of the invention can be stabilized against nucleolytic degradation such as by the incorporation of a modification, e.g., a nucleotide modification. For example, nucleic acid sequences of the invention includes a phosphorothioate at least the first, second, or third internucleotide linkage at the 5′ or 3′ end of the nucleotide sequence. As another example, the nucleic acid sequence can include a 2-modified nucleotide, e.g., a 2′-deoxy, 2′-deoxy-2′-fluoro, 2-O-methyl, 2′-O-methoxyethyl (2-O-MOE), 2′-O-aminopropyl (2-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O—NMA). As another example, the nucleic acid sequence can include at least one 2′-O-methyl-modified nucleotide, and in some embodiments, all of the nucleotides include a 2′-O-methyl modification. In some embodiments, the nucleic acids are “locked,” i.e., comprise nucleic acid analogues in which the ribose ring is “locked” by a methylene bridge connecting the 2′-0 atom and the 4′-C atom (see, e.g., Kaupinnen et al., Drug Disc. Today 2(3):287-290 (2005); Koshkin et al., J. Am. Chem. Soc., 120(50):13252-13253 (1998)). For additional modifications see US 20100004320, US 20090298916, and US 20090143326.

It is understood that any of the modified chemistries or formats of inhibitory nucleic acids described herein can be combined with each other, and that one, two, three, four, five, or more different types of modifications can be included within the same molecule.

Techniques for the manipulation of nucleic acids used to practice this invention, such as, e.g., subcloning, labeling probes (e.g., random-primer labeling using Klenow polymerase, nick translation, amplification), sequencing, hybridization and the like are well described in the scientific and patent literature, see, e.g., Sambrook et al., Molecular Cloning: A Laboratory Manual, 3d. ed., 2001, Cold Spring Harbor Laboratory Press, New York; Ausubel et al., eds., Current Protocols in Molecular Biology, 2010, John Wiley & Sons, Inc., New York; Kriegler Gene Transfer and Expression: A Laboratory Manual, 1990; Laboratory Techniques In Biochemistry And Molecular Biology: Hybridization With Nucleic Acid Probes, Part I. Theory and Nucleic Acid Preparation, 1993, Tijssen, ed. Elsevier, New York.

Programmable RNA-Guided Nuclease Systems

In some embodiments, the HERV-K RT blocking agent comprises a programmable RNA-guided nuclease system that edits or disrupts a gene encoding a HERV-K RT (e.g., a HERV-K pol gene). Relevant genome editing techniques include, but are not limited to: zinc finger nucleases, transcription activator-like effector nucleases (TALENs), CRISPR from Prevotella and Francisella 1 (Cpf1) nucleases, meganucleases, and CRISPR/Cas9 nuclease systems. As used herein, the term “edits” in reference to a programmable RNA-guided nuclease system includes mutations such as, a point mutation, an insertion, a deletion, a frameshift, or a missense mutation at a target nucleic acid.

In some embodiments, the HERV-K RT blocking agent comprises a programmable RNA-guided nuclease system that specifically targets a nucleic acid encoding a HERV-K RT. In some embodiments the nucleic acid is an RNA molecule. In some embodiments, the nucleic acid is a DNA molecule. The RNA-guided nuclease system includes guide RNAs comprising a sequence that is complementary to the sequence of a nucleic acid (i.e., a targeting domain) in the gene encoding a HERV-K RT, and a sequence (e.g., a PAM sequence) that is targetable by a nuclease molecule (e.g., a Cas9 molecule). Upon successful targeting, the nuclease molecule cleaves the targeted nucleic acid.

The components of a nuclease system may be delivered to a subject as proteins, nucleic acids, or a combination of both.

In some embodiments, the HERV-K RT blocking agent includes guide RNAs directing the editing enzyme (e.g., a Cas9 enzyme) to a nucleic acid encoding a HERV-K RT, i.e., comprising a sequence that is complementary to the sequence of a nucleic acid encoding a HERV-K RT, and that include a PAM sequence that is targetable by a co-administered nuclease (e.g., a Cas9 enzyme).

In some embodiments, the HERV-K RT blocking agent comprises a CRISPR/Cas9 nuclease system comprises a Cas9 molecule and a guide RNA that targets the Cas9 molecule to a nucleic acid encoding a HERV-K RT. Preferably a single guide RNA (sgRNA) is used, though a crRNA/tracrRNA pair can also be used. In some embodiments, the HERV-K RT blocking agent comprises a catalytically inactive or “dead” Cas9 (dCas9) molecule fused to a Krüppel-associated box (KRAB) domain of Kox1 (see e.g., Gilbert et al. Cell 154(2): 442-51, incorporated herein by reference), and a guide RNA that targets the Cas9 molecule to a gene encoding a HERV-K RT, which silences or reduces the expression of the gene.

The sequences of multiple Cas9 molecules, as well as their respective PAM sequences, are known in the art (see, e.g., Kleinstiver et al. (2015) Nature 523 (7561): 481-5; Hou et al. (2013) Proc. Natl. Acad. Sci. U.S.A. 110(39): 15644-9; Fonfara et al. (2014) Nucleic Acids Res. 42: 2577-90; Esvelt et al. (2013) Nat. Methods 10: 1116-21; Cong et al. (2013) Science 339: 819-23; and Horvath et al. (2008) J. Bacteriol. 190: 1401-12; Abudayyeh et al. (2017) Nature 550: 280-84; PCT Publication Nos. WO 2016/141224, WO 2014/204578, and WO 2014/144761; U.S. Pat. No. 9,512,446; and US Publication No. 2014/0295557; the entire contents of each of which are incorporated herein by reference). In some embodiments, the Cas9 molecule is a Streptococcus pyogenes Cas9 molecule (spCas9). Variants of the SpCas9 system can also be used (e.g., truncated sgRNAs (Tsai et al. (2015) Nat. Biotechnol. 33: 187-97; Fu et al. (2014) Nat. Biotechnol. 32: 279-84), nickase mutations (Mali et al. (2013) Nat. Biotechnol. 31: 833-8 (2013); Ran et al. (2013) Cell 154: 1380-9), FokI-dCas9 fusions (Guilinger et al. (2014) Nat. Biotechnol. 32: 577-82; Tsai et al. (2014) Nat. Biotechnol. 32: 569-76; and PCT Publication No. WO 2014/144288; the entire contents of each of which are incorporated herein by reference). The nucleases can include one or more of SpCas9 D1135E variant; SpCas9 VRER variant; SpCas9 EQR variant; SpCas9 VQR variant; Streptococcus thermophilus Cas9 molecule (StCas9); Treponema denticola Cas9 molecule (TdCas9); or Neisseria meningitidis Cas9 molecule (NmCas9), as well as variants thereof that are at least 80%, 85%, 90%, 95%, 96%, 97%, 98%, 99% or 100% identical thereto that retain at least one function of the enzyme from which they are derived, e.g., the ability to complex with a gRNA, bind to target DNA specified by the gRNA, and alter the sequence (e.g., cleave) of the target DNA.

In some embodiments, the HERV-K RT blocking agent comprises a Cpf1 nuclease system comprising a Cpf1 nuclease molecule and a guide RNA that targets the Cpf1 nuclease molecule to a nucleic acid encoding a HERV-K RT. Cpf1 is a Cas protein that can be programmed to cleave target DNA molecules (Zetsche et al. (2015) Cell 163: 759-71; Schunder et al. (2013) Int. J. Med. Microbiol. 303: 51-60; Makarova et al. (2015) Nat. Rev. Microbiol. 13: 722-36; Fagerlund et al. (2015) Genome Biol. 16: 251). In some embodiments, the Cpf1 nuclease molecule is Acidaminococcus sp. BV3L6 (AsCpf1; NCBI Reference Sequence: WP_021736722.1), or a variant thereof. In some embodiments, the Cpf1 nuclease molecule is Lachnospiraceae bacterium ND2006 (LbCpf1; GenBank Accession No. WP_051666128.1) or a variant thereof. Unlike SpCas9, Cpf1 requires only a single 42-nt crRNA, which has 23 nt at its 3′ end that are complementary to the protospacer of the target DNA sequence (Zetsche et al. (2015)). Furthermore, whereas SpCas9 recognizes an NGG PAM sequence that is 3′ of the protospacer, AsCpf1 and LbCp1 recognize TTTN PAMs that are found 5′ of the protospacer (Zetsche et al. (2015)). In some embodiments, the Cpf1 nuclease molecule is a variant of a wild-type Cpf1 molecule that is at least 80%, 85%, 90%, 95%, 96%, 97%, 98%, 99% or 100% identical to a wild-type Cpf1 nuclease molecule, and retains at least one function of the enzyme from which it was derived, e.g., the ability to complex with a gRNA, bind to target DNA specified by the gRNA, and/or alter the sequence (e.g., cleave) of the target DNA.

In some embodiments, the HERV-K RT blocking agent comprises a CRISPR/CAS13 nuclease system comprising a CRISPR/Cas13 nuclease molecule and a guide RNA that targets the CRISPR/Cas13 molecule to a nucleic acid encoding a HERV-K RT. CRISPR/Cas13 molecules can be programmed to cleave target RNA molecules (e.g., mRNA) (see, e.g., Abudayyeh et al. (2017) Nature 550: 280-84). In some embodiments the CRISPR/Cas13 nuclease molecule is Casl3a from Leptotrichia wadei (LwaCas13a). In some embodiments, the Cas13 nuclease molecule is a variant of a wild-type LwaCas13a that is at least 80%, 85%, 90%, 95%, 96%, 97%, 98%, 99% or 100% identical to a wild-type LwaCas13a, and retains at least one function of the enzyme from which it was derived, e.g., the ability to complex with a gRNA, bind to target RNA specified by a gRNA, and/or alter the sequence (e.g., cleave) of the target RNA. To determine the percent identity of two sequences, the sequences are aligned for optimal comparison purposes (gaps are introduced in one or both of a first and a second amino acid or nucleic acid sequence as required for optimal alignment, and non-homologous sequences can be disregarded for comparison purposes). The length of a reference sequence aligned for comparison purposes is at least 80% (in some embodiments, about 85%, 90%, 95%, or 100% of the length of the reference sequence) is aligned. The nucleotides or residues at corresponding positions are then compared. When a position in the first sequence is occupied by the same nucleotide or residue as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. For example, the percent identity between two amino acid sequences can be determined using the Needleman and Wunsch algorithm (see Needleman and Wunsch (1970) J. Mol. Biol. 48: 444-53) which has been incorporated into the GAP program in the GCG software package, using a Blossum 62 scoring matrix with a gap penalty of 12, a gap extend penalty of 4, and a frameshift gap penalty of 5.

When the HERV-K RT blocking agent comprising a RNA-guided nuclease system is delivered as nucleic acids, expression constructs may be used. Expression constructs encoding one or both of guide RNAs and/or Cas9 editing enzymes can be administered in any effective carrier, e.g., any formulation or composition capable of effectively delivering the component gene to cells in vivo, in vitro, or ex vivo.

A preferred approach for in vivo introduction of nucleic acid into a cell is by use of a viral vector containing nucleic acid, e.g., a cDNA. Infection of cells with a viral vector has the advantage that a large proportion of the targeted cells can receive the nucleic acid. Additionally, molecules encoded within the viral vector, e.g., by a cDNA contained in the viral vector, are expressed efficiently in cells that have taken up viral vector nucleic acid.

In some embodiments, nucleic acids encoding a RNA-guided programmable nuclease system targeting a nucleic acid encoding a HERV-K RT (e.g., Cas9 and/or gRNA) are entrapped in liposomes bearing positive charges on their surface (e.g., lipofectins). These delivery vehicles can also be used to deliver Cas9 protein/gRNA complexes.

In clinical settings, the RNA-guided programmable nuclease systems can be introduced into a subject by any of a number of methods, each of which is familiar in the art. In some embodiments, the nucleic acids encoding a RNA-guided programmable nuclease system are administered during or after a surgical procedure; in some embodiments, a controlled-release hydrogel comprising the nucleic acids encoding a RNA-guided programmable nuclease system is administered to provide a steady dose of the nucleic acids encoding RNA-guided programmable nuclease system over time.

A pharmaceutical preparation of the nucleic acids encoding a RNA-guided programmable nuclease system can consist essentially of the gene delivery system (e.g., viral vector(s)) in an acceptable diluent, or can comprise a slow release matrix in which the gene delivery vehicle is embedded. Alternatively, where the complete gene delivery system can be produced intact from recombinant cells, e.g., retroviral vectors, the pharmaceutical preparation can comprise one or more cells, which produce the gene delivery system. In some embodiments, adeno-associated virus 1 (AAV1) vectors are used as a recombinant gene delivery system for the transfer and expression of the RNA-guided programmable nuclease system in vivo, particularly into humans. These vectors provide efficient delivery of genes into cells, and in some cases the transferred nucleic acids are stably integrated into the chromosomal DNA of the host. Protocols for producing recombinant viruses and for infecting cells in vitro or in vivo with such viruses can be found in Ausubel, et al., eds., Gene Therapy Protocols Volume 1: Production and In Vivo Applications of Gene Transfer Vectors, Humana Press, (2008), pp. 1-32 and other standard laboratory manuals. A variety of nucleic acids have been introduced into different cell types using AAV vectors (see, e.g., the references cited above and those cited in Asokan et al., (2012) Molecular Therapy 20: 699-708; and Hermonat et al. (1984) Proc. Natl. Acad. Sci. U.S.A. 81: 6466-70; Tratschin et al. (1985) Mol. Cell. Biol. 4: 2072-81; Wondisford et al. (1988) Mol. Endocrinol. 2: 32-39; Tratschin et al. (1984) J. Virol. 51: 611-9; and Flotte et al. (1993) J. Biol. Chem. 268: 3781-90).

Preferably, the RNA-guided programmable nuclease system is specific, i.e., induces genomic alterations preferentially at the target site (i.e., a nucleic acid encoding a HERV-K RT), and does not induce alterations at other sites, or only rarely induces alterations at other sites.

Anti-HERV-K RT Antibodies

In some embodiments, the HERV-K RT blocking agent comprises an anti-HERV-K RT antibody, or an antigen-binding portion thereof, that specifically binds to a HERV-K RT. In some embodiments, the anti-HERV-K antibody, or antigen-binding portion thereof reduces and/or blocks an activity (e.g., a polymerase, integrase, or ribonuclease activity (e.g., RNAse H activity) of a HERV-K RT. In some embodiments, the anti-HERV-K antibody reduces and/or blocks the ability of a HERV-K RT to bind DNA. Exemplary antibodies that specifically bind to a HERV-K RT are known in the art and are disclosed, for example at Manghera et al. (2017) Viruses 7(1): 320-332 (ERVK2 polyclonal antibody (A01), Abnova Corp.); Tyagi et al. (2017) Retrovirology 14(1): 21; and Langat et al. (1999) J. Reprod. Immunol. 42(1): 41-58.

The term “antibody” as used herein refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments the antibody has effector function and can fix complement. In some embodiments, the antibody has reduced or no ability to bind an Fc receptor. For example, the antibody can be an isotype or subtype, fragment or other mutant, which does not support binding to an Fc receptor, e.g., it has a mutagenized or deleted Fc receptor binding region.

Methods for making antibodies and fragments thereof are known in the art, see, e.g., Harlow et al., editors, Antibodies: A Laboratory Manual (1988); Goding, Monoclonal Antibodies: Principles and Practice, (N.Y Academic Press 1983); Howard and Kaser, Making and Using Antibodies: A Practical Handbook (CRC Press; 1st edition, Dec. 13, 2006); Kontermann and Dubel, Antibody Engineering Volume 1 (Springer Protocols) (Springer; 2nd ed., May 21, 2010); Lo, Antibody Engineering: Methods and Protocols (Methods in Molecular Biology) (Humana Press; Nov. 10, 2010); and Dubel, Handbook of Therapeutic Antibodies: Technologies, Emerging Developments and Approved Therapeutics, (Wiley-VCH; 1st edition, Sep. 7, 2010).

### Reverse Transcriptase Inhibitors (RTIs)

In some embodiments, the HERV-K RT blocking agent is a reverse transcriptase inhibitor. In some embodiments, the HERV-K RT blocking agent comprises a nucleoside analog reverse transcriptase inhibitor (NRTI). In some embodiments, the HERV-K RT blocking agent comprises a nucleotide analog reverse transcriptase inhibitor. In some embodiments, the HERV-K RT blocking agent comprises a non-nucleoside reverse transcriptase inhibitor (NNRTI). In some embodiments, the HERV-K RT blocking agent comprises a combination of nucleoside analog reverse transcriptase inhibitors, nucleotide analog reverse transcriptase inhibitors, and/or non-nucleoside reverse transcriptase inhibitors. In some embodiments, the HERV-K RT blocking agent is not a NRTI.

Numerous reverse transcriptase inhibitors are known in the art, and may be used as described herein, including zidovudine (ZDV), didanosine (ddI), stavudine (d4T), zalcitabine (DDC), lamivudine (3TC), abacavir (ABC), tenofovir disoproxil (TDF), emtricitabine (FTC), etravirine lobucavir, entecavir (ETV), apricitabine, censavudine, dexelvucitabine, alovudine, amdoxovir, elvucitabine, racivir, and stampidine. Additional reverse transcriptase inhibitors are disclosed, for example, in U.S. Publication Nos. 2017/0267667, 2016/0145255, 2015/0105351, 2007/0088015, 2013/0296382, 2012/0225894, 2012/0053213, 2012/0029192, 2009/0162319, and 2007/0021442, the entire contents of each of which are incorporated herein by reference. Without wishing to be bound by any particular theory, the use of guanosine and cytidine analogs to inhibit a HERV-K RT blocking agent may be particularly advantageous in the treatment of a cancer comprising high levels of HSATII RNA given that the GC content of HSATII is high. In some embodiments, the HERV-K RT blocking agent is a cytidine analog (e.g., zalcitabine (ddC); lamivudine (3TC); and emtricitabine (FTC). In some embodiments, the HERV-K RT blocking agent is a guanosine analog (e.g., abacavir (ABC), and etecavir (ETV).

Methods of Treatment

In some embodiments, disclosed herein are methods of treating a subject in need thereof by administering a therapeutically-effective amount of an RTI. In some embodiments, disclosed herein are methods of treating a subject in need thereof by administering a therapeutically-effective amount of an NRTI. In some embodiments, disclosed herein are methods of treating a subject in need thereof by administering a therapeutically-effective amount of any one of zidovudine (ZDV), didanosine (ddI), stavudine (d4T), zalcitabine (DDC), lamivudine (3TC), abacavir (ABC), tenofovir disoproxil (TDF), emtricitabine (FTC), etravirine lobucavir, entecavir (ETV), apricitabine, censavudine, dexelvucitabine, alovudine, amdoxovir, elvucitabine, racivir, or stampidine. In some embodiments, the therapy includes administration of 3TC.

In some embodiments, the subject in need thereof has cancer. As used herein, the terms “cancer”, “hyperproliferative”, and “neoplastic” refer to cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair.

Examples of cancers that can be treated in accordance with the methods described herein include, but are not limited to, B cell lymphomas (e.g., B cell chronic lymphocytic leukemia, B cell non-Hodgkin lymphoma, cutaneous B cell lymphoma, diffuse large B cell lymphoma), basal cell carcinoma, bladder cancer, blastoma, brain metastasis, breast cancer, Burkitt lymphoma, carcinoma (e.g., adenocarcinoma (e.g., of the gastroesophageal junction)), cervical cancer, colon cancer, colorectal cancer (colon cancer and rectal cancer), endometrial carcinoma, esophageal cancer, Ewing sarcoma, follicular lymphoma, gastric cancer, gastroesophageal junction carcinoma, gastrointestinal cancer, glioblastoma (e.g., glioblastoma multiforme, e.g., newly diagnosed or recurrent), glioma, head and neck cancer (e.g., head and neck squamous cell carcinoma), hepatic metastasis, Hodgkin's and non-Hodgkin's lymphoma, kidney cancer (e.g., renal cell carcinoma and Wilms' tumors), laryngeal cancer, leukemia (e.g., chronic myelocytic leukemia, hairy cell leukemia), liver cancer (e.g., hepatic carcinoma and hepatoma), lung cancer (e.g., non-small cell lung cancer and small-cell lung cancer), lymphblastic lymphoma, lymphoma, mantle cell lymphoma, metastatic brain tumor, metastatic cancer, myeloma (e.g., multiple myeloma), neuroblastoma, ocular melanoma, oropharyngeal cancer, osteosarcoma, ovarian cancer, pancreatic cancer (e.g., pancreatis ductal adenocarcinoma), prostate cancer (e.g., hormone refractory (e.g., castration resistant), metastatic, metastatic hormone refractory (e.g., castration resistant, androgen independent)), renal cell carcinoma (e.g., metastatic), salivary gland carcinoma, sarcoma (e.g., rhabdomyosarcoma), skin cancer (e.g., melanoma (e.g., metastatic melanoma)), soft tissue sarcoma, solid tumor, squamous cell carcinoma, synovia sarcoma, testicular cancer, thyroid cancer, transitional cell cancer (urothelial cell cancer), uveal melanoma (e.g., metastatic), verrucous carcinoma, vulval cancer, and Waldenstrom macroglobulinemia.

In some embodiments, the compositions and methods disclosed herein are used to treat a patient with Barrett's esophagus (BE). Barrett's Esophagus is a condition of the esophagus that is pre-cancerous. The standard practice for diagnosing Barrett's Esophagus uses a flexible endoscopy procedure, often with the esophageal lumen insufflated with air. A normal esophagus is usually light pink in color, while the stomach appears slightly darker pink. Barrett's Esophagus usually manifests itself as regions of slightly darker pink color above the lower esophageal sphincter (LES) that separates the stomach from the esophagus.

In some embodiments, the compositions and methods disclosed herein are used to treat a patient with colorectal cancer.

In some embodiments, the RTI is administered as an active ingredient which can be combined with a carrier material to produce a single dosage form. In some embodiments, reverse transcriptase inhibitors (e.g. an NRTI) is administered to a subject at a high dose. For example, in some embodiments, the dose is 600 mg. In some embodiments, the dose is about 100 mg, about 150 mg, about 200 mg, about 300 mg, about 400 mg, about 500 mg, about 600 mg, about 700 mg, about 800 mg, about 900 mg, or about 1000 mg. In some embodiments, the does is given once daily, twice daily, three times daily, or four times daily.

In some embodiments, the RTI is administered as a tablet, a pill, or a capsule. In some embodiments, the size of the pill is a 100 mg tablet. In some embodiments, the size of the pill is a 150 mg tablet.

In some embodiments, the mode of administration is formulated for any route of administration to a subject. Specific examples of routes of administration include intranasal, oral, pulmonary, transdermal, intradermal, and parenteral. Parenteral administration, characterized by either subcutaneous, intramuscular, or intravenous injection, is also contemplated herein. Injectables can be prepared in conventional forms, either as liquid solutions or suspensions, solid forms suitable for solution or suspension in liquid prior to injection, or as emulsions.

DNA Hypomethylating Agents

In some embodiments, the methods described herein further comprise administering a DNA hypomethylating agent to a subject. DNA methylation is an epigenetic modification that regulates the silencing of gene transcription. Genomic methylation patterns may be altered in tumors (Smet et al. (2010) Epigenetics 5(3): 206-13), and may be of significance in B cell malignancies (Debatin et al. (2007) Cell 129(5): 853-5; Martin-Subero (2006) Leukemia 20(10): 1658-60). As described below, Applicants have surprisingly discovered that cancer cells which are refractory to treatment with NRTIs become sensitive to NRTIs when NRTI treatment is combined with treatment with a DNA hypomethylating agent (e.g., 5-azacytidine). Without wishing to be bound by any particular theory, treatment with a DNA hypomethylating agent is believed to activate the transcription of HSATII, which renders the cells susceptible to treatment with a NRTI.

In some embodiments, the DNA hypomethylating agent is a DNA methyltransferase inhibitor. In some embodiments, the DNA methyltransferase inhibitor is 5′-azacytidine (AZA), decitabine (DAC), cladribine (2CdA), or a combination thereof (Wyczechowska et al. (2003) Biochem Pharmacol. 65: 219-25; Yu et al. (2006) Am. J Hematol. 81(11): 864-9; and Garcia-Manero (2008) Curr. Opin. Oncol. 20(6): 705-10). Additional DNA hypomethylating agents are described, for example in U.S. Publication Nos. 2011/0218170A1, 2005/0119201, and 2015/0258068, the entire contents of each of which are incorporated herein by reference.

### Pharmaceutical Compositions

In some embodiments, the methods described herein can include the administration of pharmaceutical compositions and formulations comprising a HERV-K RT blocking agent described herein. In some embodiments, the methods described herein can include the administration of pharmaceutical compositions and formulations comprising an RTI as described herein.

In some embodiments, the compositions are formulated with a pharmaceutically acceptable carrier. The pharmaceutical compositions and formulations can be administered parenterally, topically, orally or by local administration, such as by aerosol or transdermally. The pharmaceutical compositions can be formulated in any way and can be administered in a variety of unit dosage forms depending upon the condition or disease and the degree of illness, the general medical condition of each subject, the resulting preferred method of administration and the like. Details on techniques for formulation and administration of pharmaceuticals are well described in the scientific and patent literature, see, e.g., Remington: The Science and Practice of Pharmacy, 21st ed., 2005.

In some embodiments, the pharmaceutical compositions and formulations include an RTI as disclosed herein and a hypomethylating agent. In some embodiments, the pharmaceutical compositions and formulations include an RTI as disclosed herein and a DNA hypomethylating agent as disclosed herein.

The pharmaceutical compositions and formulations can be administered as a single active agent in a pharmaceutical composition or in combination with other active agents. The compositions may be formulated for administration, in any convenient way for use in human or veterinary medicine. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Formulations of the compositions include those suitable for intradermal, inhalation, oral/nasal, topical, parenteral, rectal, and/or intravaginal administration. The formulations may conveniently be presented in unit dosage form and may be prepared by any methods well known in the art of pharmacy. The amount of active ingredient (e.g., a HERV-K blocking agent) which can be combined with a carrier material to produce a single dosage form will vary depending upon the host being treated, the particular mode of administration, e.g., intradermal, parenteral, intravenous, or via inhalation. The amount of active ingredient which can be combined with a carrier material to produce a single dosage form will generally be that amount of the compound which produces a therapeutic effect.

Pharmaceutical formulations of this invention can be prepared according to any method known to the art for the manufacture of pharmaceuticals, and can contain sweetening agents, flavoring agents, coloring agents and preserving agents. A formulation can be admixtured with nontoxic pharmaceutically acceptable excipients which are suitable for manufacture. Formulations may comprise one or more diluents, emulsifiers, preservatives, buffers, excipients, etc. and may be provided in such forms as liquids, powders, emulsions, lyophilized powders, sprays, creams, lotions, controlled release formulations, tablets, pills, gels, on patches, in implants, etc.

Pharmaceutical formulations for oral administration can be formulated using pharmaceutically acceptable carriers well known in the art in appropriate and suitable dosages. Such carriers enable the pharmaceuticals to be formulated in unit dosage forms as tablets, pills, powder, dragees, capsules, liquids, lozenges, gels, syrups, slurries, suspensions, etc., suitable for ingestion by the subject. Pharmaceutical preparations for oral use can be formulated as a solid excipient, optionally grinding a resulting mixture, and processing the mixture of granules, after adding suitable additional compounds, if desired, to obtain tablets or dragee cores. Suitable solid excipients are carbohydrate or protein fillers include, e.g., sugars, including lactose, sucrose, mannitol, or sorbitol; starch from corn, wheat, rice, potato, or other plants; cellulose such as methyl cellulose, hydroxypropylmethyl-cellulose, or sodium carboxy-methylcellulose; and gums including arabic and tragacanth; and proteins, e.g., gelatin and collagen. Disintegrating or solubilizing agents may be added, such as the cross-linked polyvinyl pyrrolidone, agar, alginic acid, or a salt thereof, such as sodium alginate. Push-fit capsules can contain active agents mixed with a filler or binders such as lactose or starches, lubricants such as talc or magnesium stearate, and, optionally, stabilizers. In soft capsules, the active agents can be dissolved or suspended in suitable liquids, such as fatty oils, liquid paraffin, or liquid polyethylene glycol with or without stabilizers.

Aqueous suspensions can contain an active agent (e.g., nucleic acid sequences of the invention) in admixture with excipients suitable for the manufacture of aqueous suspensions, e.g., for aqueous intradermal injections. Such excipients include a suspending agent, such as sodium carboxymethylcellulose, methylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

In some embodiments, oil-based pharmaceuticals are used for administration of an HERV-K RT blocking agent described herein. Oil-based suspensions can be formulated by suspending an active agent in a vegetable oil, such as arachis oil, olive oil, sesame oil or coconut oil, or in a mineral oil such as liquid paraffin; or a mixture of these. See e.g., U.S. Pat. No. 5,716,928 describing using essential oils or essential oil components for increasing bioavailability and reducing inter- and intra-individual variability of orally administered hydrophobic pharmaceutical compounds (see also U.S. Pat. No. 5,858,401). The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto (1997) J. Pharmacol. Exp. Ther. 281:93-102.

Pharmaceutical formulations can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent. In alternative embodiments, these injectable oil-in-water emulsions of the invention comprise a paraffin oil, a sorbitan monooleate, an ethoxylated sorbitan monooleate and/or an ethoxylated sorbitan trioleate.

The pharmaceutical compounds can also be administered by in intranasal, intraocular and intravaginal routes including suppositories, insufflation, powders and aerosol formulations (for examples of steroid inhalants, see e.g., Rohatagi (1995) J. Clin. Pharmacol. 35:1187-1193; Tjwa (1995) Ann. Allergy Asthma Immunol. 75:107-111). Suppositories formulations can be prepared by mixing the drug with a suitable non-irritating excipient which is solid at ordinary temperatures but liquid at body temperatures and will therefore melt in the body to release the drug. Such materials are cocoa butter and polyethylene glycols.

In some embodiments, the pharmaceutical compounds can be delivered transdermally, by a topical route, formulated as applicator sticks, solutions, suspensions, emulsions, gels, creams, ointments, pastes, jellies, paints, powders, and aerosols.

In some embodiments, the pharmaceutical compounds can also be delivered as microspheres for slow release in the body. For example, microspheres can be administered via intradermal injection of drug which slowly release subcutaneously; see Rao (1995) J. Biomater Sci. Polym. Ed. 7:623-645; as biodegradable and injectable gel formulations, see, e.g., Gao (1995) Pharm. Res. 12:857-863 (1995); or, as microspheres for oral administration, see, e.g., Eyles (1997) J. Pharm. Pharmacol. 49:669-674.

In some embodiments, the pharmaceutical compounds can be parenterally administered, such as by intravenous (IV) administration or administration into a body cavity or lumen of an organ. These formulations can comprise a solution of active agent dissolved in a pharmaceutically acceptable carrier. Acceptable vehicles and solvents that can be employed are water and Ringer's solution, an isotonic sodium chloride. In addition, sterile fixed oils can be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of active agent in these formulations can vary widely, and will be selected primarily based on fluid volumes, viscosities, body weight, and the like, in accordance with the particular mode of administration selected and the subject's needs. For IV administration, the formulation can be a sterile injectable preparation, such as a sterile injectable aqueous or oleaginous suspension. This suspension can be formulated using those suitable dispersing or wetting agents and suspending agents. The sterile injectable preparation can also be a suspension in a nontoxic parenterally-acceptable diluent or solvent, such as a solution of 1,3-butanediol. The administration can be by bolus or continuous infusion (e.g., substantially uninterrupted introduction into a blood vessel for a specified period of time).

In some embodiments, the pharmaceutical compounds and formulations can be lyophilized. Stable lyophilized formulations comprising an oligo can be made by lyophilizing a solution comprising a pharmaceutical of the invention and a bulking agent, e.g., mannitol, trehalose, raffinose, and sucrose or mixtures thereof. A process for preparing a stable lyophilized formulation can include lyophilizing a solution about 2.5 mg/mL protein, about 15 mg/mL sucrose, about 19 mg/mL NaCl, and a sodium citrate buffer having a pH greater than 5.5 but less than 6.5. See, e.g., U.S. 20040028670.

The compositions and formulations can be delivered by the use of liposomes. By using liposomes, particularly where the liposome surface carries ligands specific for target cells, or are otherwise preferentially directed to a specific organ, one can focus the delivery of the active agent into target cells in vivo. See, e.g., U.S. Pat. Nos. 6,063,400; 6,007,839; Al-Muhammed (1996) J. Microencapsul. 13:293-306; Chonn (1995) Curr. Opin. Biotechnol. 6:698-708; Ostro (1989) Am. J. Hosp. Pharm. 46:1576-1587. As used in the present invention, the term “liposome” means a vesicle composed of amphiphilic lipids arranged in a bilayer or bilayers. Liposomes are unilamellar or multilamellar vesicles that have a membrane formed from a lipophilic material and an aqueous interior that contains the composition to be delivered. Cationic liposomes are positively charged liposomes that are believed to interact with negatively charged DNA molecules to form a stable complex. Liposomes that are pH-sensitive or negatively-charged are believed to entrap DNA rather than complex with it. Both cationic and noncationic liposomes have been used to deliver DNA to cells.

Liposomes can also include “sterically stabilized” liposomes, i.e., liposomes comprising one or more specialized lipids. When incorporated into liposomes, these specialized lipids result in liposomes with enhanced circulation lifetimes relative to liposomes lacking such specialized lipids. Examples of sterically stabilized liposomes are those in which part of the vesicle-forming lipid portion of the liposome comprises one or more glycolipids or is derivatized with one or more hydrophilic polymers, such as a polyethylene glycol (PEG) moiety. Liposomes and their uses are further described in U.S. Pat. No. 6,287,860.

The formulations of the invention can be administered for prophylactic and/or therapeutic treatments. In some embodiments, for therapeutic applications, compositions are administered to a subject who is at risk of or has a disorder described herein, in an amount sufficient to cure, alleviate or partially arrest the clinical manifestations of the disorder or its complications; this can be called a therapeutically effective amount.

The amount of pharmaceutical composition adequate to accomplish this is a therapeutically effective dose. The dosage schedule and amounts effective for this use, i.e., the dosing regimen, will depend upon a variety of factors, including the stage of the disease or condition, the severity of the disease or condition, the general state of the subject's health, the subject's physical status, age and the like. In calculating the dosage regimen for a subject, the mode of administration also is taken into consideration.

The dosage regimen also takes into consideration pharmacokinetics parameters well known in the art, i.e., the active agents' rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; Remington: The Science and Practice of Pharmacy, 21st ed., 2005). The state of the art allows the clinician to determine the dosage regimen for each individual subject, active agent and disease or condition treated. Guidelines provided for similar compositions used as pharmaceuticals can be used as guidance to determine the dosage regiment, i.e., dose schedule and dosage levels, administered practicing the methods of the invention are correct and appropriate.

Single or multiple administrations of formulations can be given depending on for example: the dosage and frequency as required and tolerated by the subject, the degree and amount of therapeutic effect generated after each administration (e.g., effect on tumor size or growth), and the like. The formulations should provide a sufficient quantity of active agent to effectively treat, prevent or ameliorate conditions, diseases or symptoms (e.g., cancer).

### Combination Therapy

In some embodiments, the methods described herein further comprise administering a HERV-K blocking agent, e.g., an RTI, in combination with a second therapeutic agent selected from the group consisting of a DNA hypomethylating agent, an HSATII inhibitory nucleic acid, or an immunotherapeutic agent, as provided below.

HSATII Inhibitory Nucleic Acids

In some embodiments, the methods described herein comprise further administering to a subject an inhibitory nucleic acid (e.g., a LNA molecule) that specifically targets HSATII. Inhibitory nucleic acids targeting HSATII and methods of using the same are disclosed, e.g., in U.S. Publication No. 2017/0198288, the entire contents of which are expressly incorporated herein by reference.

Immunotherapeutic Agents

In some embodiments, the methods also include co-administering an immunotherapy agent to a subject who is treated with a method or composition described herein. Immunotherapy agents include those therapies that target tumor-induced immune suppression; see, e.g., Stewart and Smyth (2011) Cancer Metastasis Rev. 30(1): 125-40.

Examples of immunotherapies include, but are not limited to, adoptive T cell therapies or cancer vaccine preparations designed to induce T lymphocytes to recognize cancer cells, as well as checkpoint inhibitors such as anti-CD137 antibodies (e.g., BMS-663513), anti-PD1 antibodies (e.g., Nivolumab, pembrolizumab/MK-3475, Pidilizumab (CT-011)), anti-PDL1 antibodies (e.g., BMS-936559, MPDL3280A), or anti-CTLA-4 antibodies (e.g., ipilumimab; see, e.g., Krüger et al. (2007) Histol Histopathol. 22(6): 687-96; Eggermont et al. (2010) Semin Oncol. 37(5): 455-9; Klinke (2010) Mol. Cancer. 9: 242; Alexandrescu et al. (2010) J. Immunother. 33(6): 570-90; Moschella et al. (2010) Ann NY Acad Sci. 1194: 169-78; Ganesan and Bakhshi (2010) Natdl Med. J. India 23(1): 21-7; and Golovina and Vonderheide (2010) Cancer J. 16(4): 342-7.

Exemplary anti-PD-1 antibodies that can be used in the methods described herein include those that bind to human PD-1; an exemplary PD-1 protein sequence is provided at NCBI Accession No. NP_005009.2. Exemplary antibodies are described in U.S. Pat. Nos. 8,008,449; 9,073,994; and U.S. Publication No. 2011/0271358, including, e.g., PF-06801591, AMP-224, BGB-A317, BI 754091, JS001, MEDI0680, PDR001, REGN2810, SHR-1210, TSR-042, pembrolizumab, nivolumab, avelumab, pidilizumab, and atezolizumab.

Exemplary anti-CD40 antibodies that can be used in the methods described herein include those that bind to human CD40; exemplary CD40 protein precursor sequences are provided at NCBI Accession No. NP_001241.1, NP_690593.1, NP_001309351.1, NP_001309350.1 and NP_001289682.1. Exemplary antibodies include those described in International Publication Nos. WO 2002/088186; WO 2007/124299; WO 2011/123489; WO 2012/149356; WO 2012/111762; WO 2014/070934; U.S. Publication Nos. 2013/0011405; 2007/0148163; 2004/0120948; 2003/0165499; and U.S. Pat. No. 8,591,900; including, e.g., dacetuzumab, lucatumumab, bleselumab, teneliximab, ADC-1013, CP-870,893, Chi Lob 7/4, HCD122, SGN-4, SEA-CD40, BMS-986004, and APX005M. In some embodiments, the anti-CD40 antibody is a CD40 agonist, and not a CD40 antagonist.

Exemplary anti-PD-L1 antibodies that can be used in the methods described herein include those that bind to human PD-L1; exemplary PD-L1 protein sequences are provided at NCBI Accession No. NP_001254635.1, NP_001300958.1, and NP_054862.1. Exemplary antibodies are described in U.S. Publication No. 2017/0058033; International Publication Nos. WO 2016/061142A1; WO 2016/007235A1; WO 2014/195852A1; and WO 2013/079174A1, including, e.g., BMS-936559 (MDX-1105), FAZ053, KN035, Atezolizumab (Tecentriq, MPDL3280A), Avelumab (Bavencio), and Durvalumab (Imfinzi, MEDI-4736).

In some embodiments, these immunotherapies may primarily target immunoregulatory cell types such as regulatory T cells (Tregs) or M2 polarized macrophages, e.g., by reducing number, altering function, or preventing tumor localization of the immunoregulatory cell types. For example, Treg-targeted therapy includes anti-GITR monoclonal antibody (TRX518), cyclophosphamide (e.g., metronomic doses), arsenic trioxide, paclitaxel, sunitinib, oxaliplatin, PLX4720, anthracycline-based chemotherapy, Daclizumab (anti-CD25); immunotoxin e.g., Ontak (denileukin diftitox); lymphoablation (e.g., chemical or radiation lymphoablation) and agents that selectively target the VEGF-VEGFR signalling axis, such as VEGF blocking antibodies (e.g., bevacizumab), or inhibitors of VEGFR tyrosine kinase activity (e.g., lenvatinib) or ATP hydrolysis (e.g., using ectonucleotidase inhibitors, e.g., ARL67156 (6-N,N-Diethyl-D-β,γ-dibromomethyleneATP trisodium salt), 8-(4-chlorophenylthio) cAMP (pCPT-cAMP) and a related cyclic nucleotide analog (8-[4-chlorophenylthio] cGMP; pCPT-cGMP) and those described in International Publication No. WO 2007/135195, as well as monoclonal antibodies (mAbs) against CD73 or CD39). Docetaxel also has effects on M2 macrophages. See, e.g., Zitvogel et al. (2013) Immunity 39: 74-88.

In some embodiments, the methods also include co-administering a chemotherapeutic agent. In some embodiments, the chemotherapeutic agent is a toxin or cytotoxic drug, including but not limited to ricin, modified Pseudomonas enterotoxin A, calicheamicin, adriamycin, 5-fluorouracil, and the like. In some embodiments, the chemotherapeutic agent is fluorouracil (5FU or 5-FU).

In another example, M2 macrophage targeted therapy includes clodronate-liposomes (Zeisberger, et al. (2006) Br. J. Cancer 95: 272-81), DNA based vaccines (Luo et al. (2006) J. Clin. Invest. 116(8): 2132-41), and M2 macrophage targeted pro-apoptotic peptides (Cieslewicz et al. (2013) Proc. Natl. Acad. Sci. U.S.A. 110(40): 15919-24). Some useful immunotherapies target the metabolic processes of immunity, and include adenosine receptor antagonists and small molecule inhibitors, e.g., istradefylline (KW-6002) and SCH-58261; indoleamine 2,3-dioxygenase (IDO) inhibitors, e.g., small molecule inhibitors (e.g., 1-methyl-tryptophan (1MT), 1-methyl-d-tryptophan (D1MT), and Toho-1) or IDO-specific siRNAs, or natural products (e.g., brassinin or exiguamine) (see, e.g., Munn (2012) Front. Biosci. (Elite Ed).4: 734-45) or monoclonal antibodies that neutralize the metabolites of IDO, e.g., mAbs against N-formyl-kynurenine.

In some embodiments, the immunotherapies may antagonize the action of cytokines and chemokines such as IL-10, TGF-β, IL-6, CCL2 and others that are associated with immunosuppression in cancer. For example, TGF-β neutralizing therapies include anti-TGF-β antibodies (e.g. fresolimumab, infliximab, lerdelimumab, GC-1008), antisense oligodeoxynucleotides (e.g., trabedersen), and small molecule inhibitors of TGF-beta (e.g. LY2157299) (Wojtowicz-Praga (2003) Invest. New Drugs 21(1): 21-32). Another example of therapies that antagonize immunosuppression cytokines can include anti-IL-6 antibodies (e.g. siltuximab) (Guo, et al., Cancer Treat Rev. 38(7):904-910 (2012). mAbs against IL-10 or its receptor can also be used, e.g., humanized versions of those described in Llorente et al., Arthritis & Rheumatism, 43(8): 1790-1800, 2000 (anti-IL-10 mAb), or Newton et al., Clin Exp Immunol. 2014 July; 177(1):261-8 (anti-interleukin-10R1 monoclonal antibody). mAbs against CCL2 or its receptors can also be used. In some embodiments, the cytokine immunotherapy is combined with a commonly used chemotherapeutic agent (e.g., gemcitabine, docetaxel, cisplatin, tamoxifen) as described in U.S. Pat. No. 8,476,246.

In some embodiments, immunotherapies can include agents that are believed to elicit “danger” signals, e.g., “PAMPs” (pathogen-associated molecular patterns) or “DAMPs” (damage-associated molecular patterns) that stimulate an immune response against the cancer. See, e.g., Pradeu and Cooper (2012) Front Immunol. 3: 287; Escamilla-Tilch et al. (2013) Immunol. Cell. Biol. 91(10): 601-10. In some embodiments, immunotherapies can agonize toll-like receptors (TLRs) to stimulate an immune response. For example, TLR agonists include vaccine adjuvants (e.g., 3M-052) and small molecules (e.g., imiquimod, muramyl dipeptide, CpG, and mifamurtide (muramyl tripeptide)), as well as polysaccharide krestin and endotoxin. See, Galluzi et al. (2012) Oncoimmunol. 1(5): 699-716, Lu et al. (2011) Clin. Cancer Res. 17(1): 67-76, and U.S. Pat. Nos. 8,795,678 and 8,790,655. In some embodiments, immunotherapies can involve administration of cytokines that elicit an anti-cancer immune response, see Lee & Margolin (2011) Cancers 3: 3856-93. For example, the cytokine IL-12 can be administered (Portielje, et al. (2003) Cancer Immunol. Immunother. 52: 133-44) or as gene therapy (Melero et al. (2001) Trends Immunol. 22(3): 113-5). In another example, interferons (IFNs), e.g., IFNgamma, can be administered as adjuvant therapy (Dunn et al. (2006) Nat. Rev. Immunol. 6: 836-48).

In some embodiments, immunotherapies can antagonize cell surface receptors to enhance the anti-cancer immune response. For example, antagonistic monoclonal antibodies that boost the anti-cancer immune response can include antibodies that target CTLA-4 (ipilimumab, see Tarhini and Iqbal (2010) Onco Targets Ther. 3: 15-25 and U.S. Pat. No. 7,741,345, or tremelimumab) or antibodies that target PD-1 (nivolumab, see Topalian et al. (2012) N. Engl. J Med. 366(26): 2443-54 and International Publication No. WO 2013/173223, pembrolizumab/MK-3475, and pidilizumab (CT-011)).

Some immunotherapies enhance T cell recruitment to the tumor site (such as endothelin receptor-A/B (ETRA/B) blockade, e.g., with macitentan or the combination of the ETRA and ETRB antagonists BQ123 and BQ788, see Coffman et al. (2013) Cancer Biol Ther. 14(2): 184-92), or enhance CD8 T-cell memory cell formation (e.g., using rapamycin and metformin, see, e.g., Pearce et al. (2009) Nature 460(7251): 103-7; Mineharu et al. (2014) Mol. Cancer Ther. 13(12): 3024-36; and Berezhnoy et al. (2014) Oncoimmunology 3: e28811). Immunotherapies can also include administering one or more of: adoptive cell transfer (ACT) involving transfer of ex vivo expanded autologous or allogeneic tumor-reactive lymphocytes, e.g., dendritic cells or peptides with adjuvant; cancer vaccines such as DNA-based vaccines, cytokines (e.g., IL-2), cyclophosphamide, anti-interleukin-2R immunotoxins, and/or prostaglandin E2 inhibitors (e.g., using SC-50). In some embodiments, the methods include administering a composition comprising tumor-pulsed dendritic cells, e.g., as described in International Publication No. WO 2009/114547 and references cited therein. See also Shiao et al. (2011) Genes & Dev. 25: 2559-72.

Combination Therapy with Hypomethylating Agents

In some embodiments, disclosed herein are methods of treatment comprising administering to the subject a hypomethylating agent with an RTI. In some embodiments, the hypomethylating agent is a DNA and histone methylation inhibitor. In some embodiments, the hypomethylating agent is a DNA hypomethylating agent. In some embodiments, the DNA hypomethylating agent is a DNA methyltransferase inhibitor. In some embodiments, the DNA methyltransferase inhibitor is 5′-azacytidine (AZA), decitabine (DAC), cladribine (2CdA), or a combination thereof (Wyczechowska et al. (2003) Biochem Pharmacol. 65: 219-25; Yu et al. (2006) Am. J. Hematol. 81(11): 864-9; and Garcia-Manero (2008) Curr. Opin. Oncol. 20(6): 705-10). Additional DNA hypomethylating agents are described, for example in U.S. Publication Nos. 2011/0218170A1, 2005/0119201, and 2015/0258068, the entire contents of each of which are incorporated herein by reference.

In some embodiments, also disclosed herein are methods of treatment comprising administering to the subject a histone deacetylase inhibitor (“HDAC inhibitor” or “HDACi”) with an RTI. In some embodiments, the HDAC inhibitor is one of entinostat, panobinostat, or vorinostat.

### EXAMPLES

The invention is further described in the following examples, which do not limit the scope of the invention described in the claims.

### Example 1: Treatment of Cancer Cells with Antisense “Locked” Nucleic Acids Complementary to HSATIT RNA Induces Cell Death

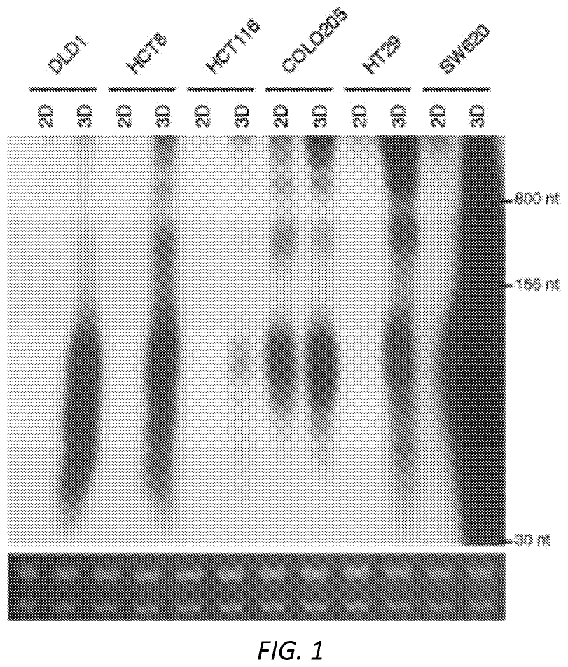

Satellite repeats are non-protein coding regions of genomic DNA that make up a large portion of mammalian genomes. While these genomic regions are normally transcriptionally silent (i.e., not transcribed into RNA), they can be transcribed under certain conditions, particularly in cancer cells. Applicants have discovered that the satellite repeat RNA HSATII is expressed at high levels in cancer cells (e.g., epithelial cancer cells), under non-adherent 3D growth conditions, but not in adherent 2D conditions (FIG. 1). HSATII is an attractive drug target because it is exclusively expressed in tumors in most types of cancer (see Ting et al. (2011) Science 331(6017): 593-6; and Bersani et al. (2015) Proc. Natl. Acad. Sci. U.S.A. 112(49): 15148-53).

In order to target HSATII in cancer cells, antisense “locked” nucleic acids (LNAs) that are complimentary to the HSATII RNA sequence were used. COL0205 human colorectal cancer cells were transfected with either a scrambled LNA (5′-AACACGTCTATACGC-3′ (SEQ ID NO: 5), or LNAs targeting HSATII (LNA1: 5′-GATTCCATTCGATGAT-3′ (SEQ ID NO: 6); LNA2: 5′-+A*+T*+G*+G*A*A*T*C*A*T*C*A*T*+C*+G*+A*+A-3′ (SEQ ID NO: 10; *=phosphorothioate bond; +=locked nucleic acid base); LNA3: 5′-+T*+G*+G*A*A*T*C*A*T*/iMe-dC/G*A*A*T*+G*+G*+A-3′ (SEQ ID NO: 11 (*=phosphorothioate bond; +=locked nucleic acid base; * iMe-dC=5-methyl deoxycytidine)). RNA was isolated using TRIZOL at days 1-6 post transfection. Purified RNA was subjected to digital gene expression (DGE) sample prepping and analysis on a HeliScope Single Molecule Sequencer (formerly Helicos BioSciences Corp.; now SeqLL, LLC, Woburn, Mass.). Briefly, single stranded cDNA was reverse transcribed from RNA with a dTU25V primer (a modified version of oligo-dT priming) and the Superscript III cDNA synthesis kit (Invitrogen™/Life Technologies™). Purified single stranded cDNA was denatured and then a poly-A tail was added to the 3′ end using terminal transferase (New England Biolabs©).

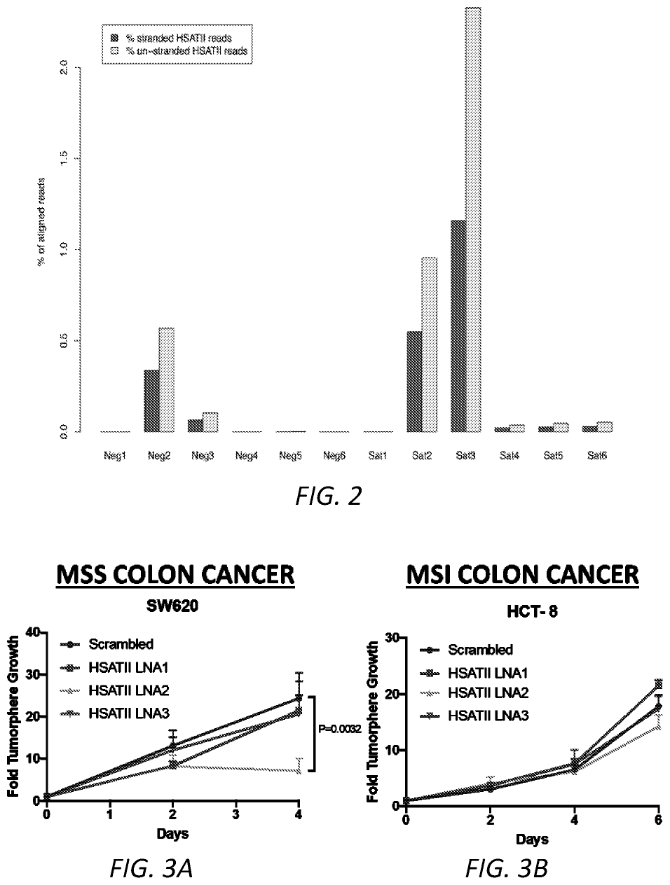

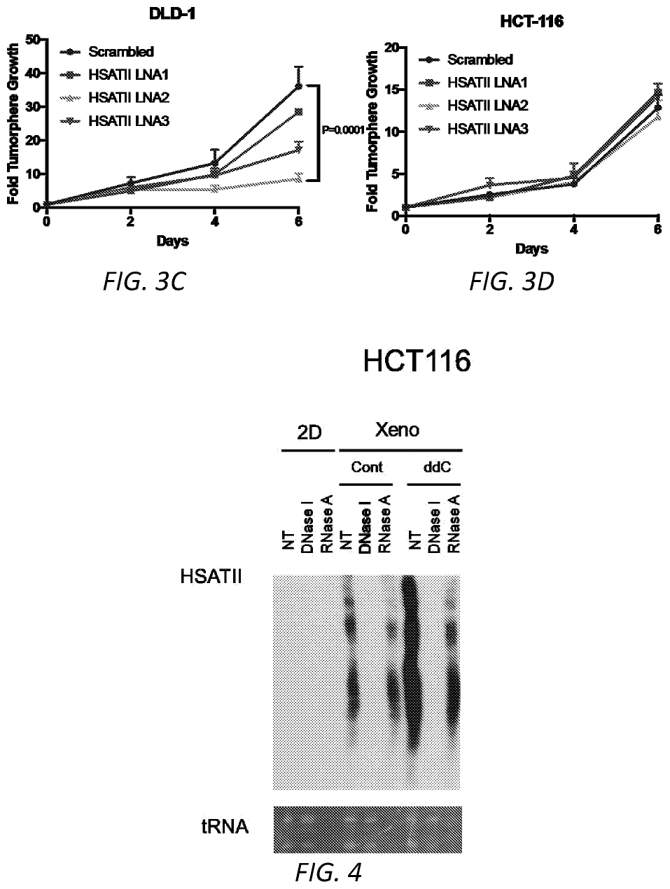

As shown in FIG. 2, LNAs complementary to HSATII RNA resulted in the accumulation of HSATII RNA in the cells. Moreover, treatment with LNAs caused cell death in colorectal cancer cell line 3D tumorspheres (see FIGS. 3A-3D). Interestingly, cell death was observed solely in the Microsatellite Stable (MSS) colon cancer cell lines SW620 and DLD-1, but not in the Microsatellite Instable (MSI) colon cancer cell lines HACT-8 and HCT-116.

### Example 2: Treatment of Colorectal Cancer Cells with Nucleoside Reverse Transcriptase Inhibitors (NRTIs) Induces Cell Death

Previous studies have shown that HSATII RNA has a high degree of sequence similarity with viral RNA, and the presence of this RNA can trigger an innate immune response in cells similar to that which is seen in response to infection by viruses (Tanne et al. (2015) Proc. Natl. Acad. Sci. U.S.A. 112(49): 15154-9). HSATII RNA is also reverse transcribed and reintegrated into the genome, which leads to genomic expansion (Bersani et al. (2015) Proc. Natl. Acad. Sci. U.S.A. 112(49): 15148-53). This behavior is not commonly associated with mammalian RNA, but is a defining feature of retroviruses such as the human immunodeficiency virus (HIV). It is widely accepted that in retroviruses one of the functions of reverse transcription is to escape detection by the innate immune system in infected cells, as DNA is less immunogenic than RNA. For example, the reverse transcribed DNA of HIV is integrated into the host genome, making it undetectable by the immune system. Thus, it is possible that HSATII reverse transcription plays a similar functional role in cancer cells. To test this hypothesis, cancer cell lines were treated with antiretroviral nucleoside reverse transcriptase inhibitors (NRTIs) to target HSATII. The NRTI dideoxycytidine (ddC) was administered to mice having HCT 116 xenografts at a dose of 25 mg/kg. As control, mice were administered vehicle only (DMSO). RNA was purified and analyzed by Northern blot. Total RNA (5 μg) before or after nuclease treatment (DNase I or RNase A) was electrophoresed in a 4% or 8% polyacrylamide-urea gel and transferred by electroblotting onto Hybond-N+ membrane (Amersham/GE Healthcare). Hybridization was performed with the following ³²P-labeled DNA oligos: anti-HSATII S, 5′-CATTCGATTCCATTCGATGAT-3′ (SEQ ID NO: 3). As shown in FIG. 4, NRTI (ddC) treatment led to the accumulation of HSATII RNA in colorectal cancer cells grown in 3D xenograft tumors. The non-treated (“NT”) RNA is higher between control (“Cont”) vs ddC treated xenograft (“Xeno”). RNase A treatment abrogated half of the signal (last lane) indicating that HSATII RNA accumulated in ddC treated tumors.

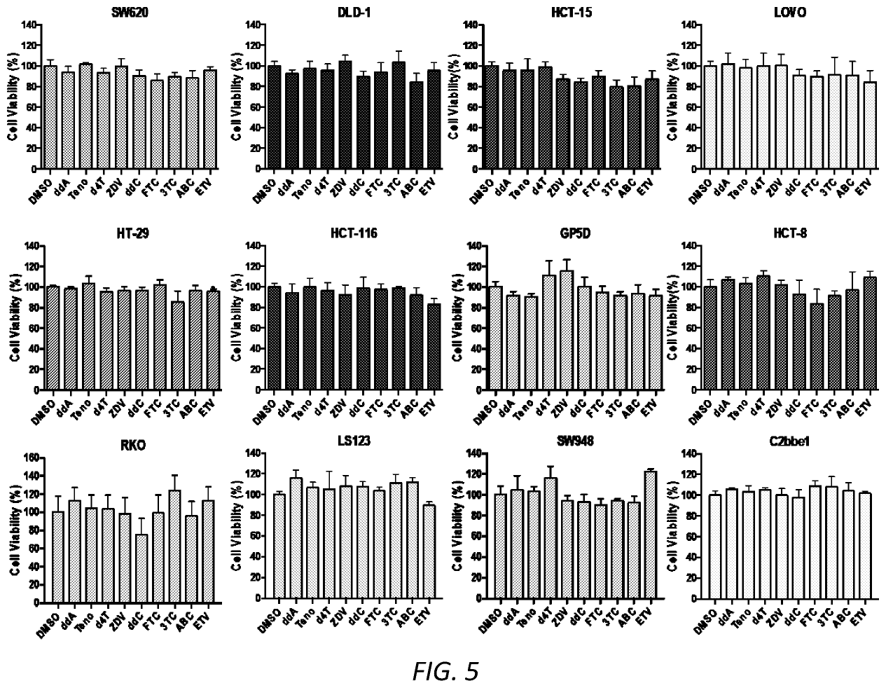

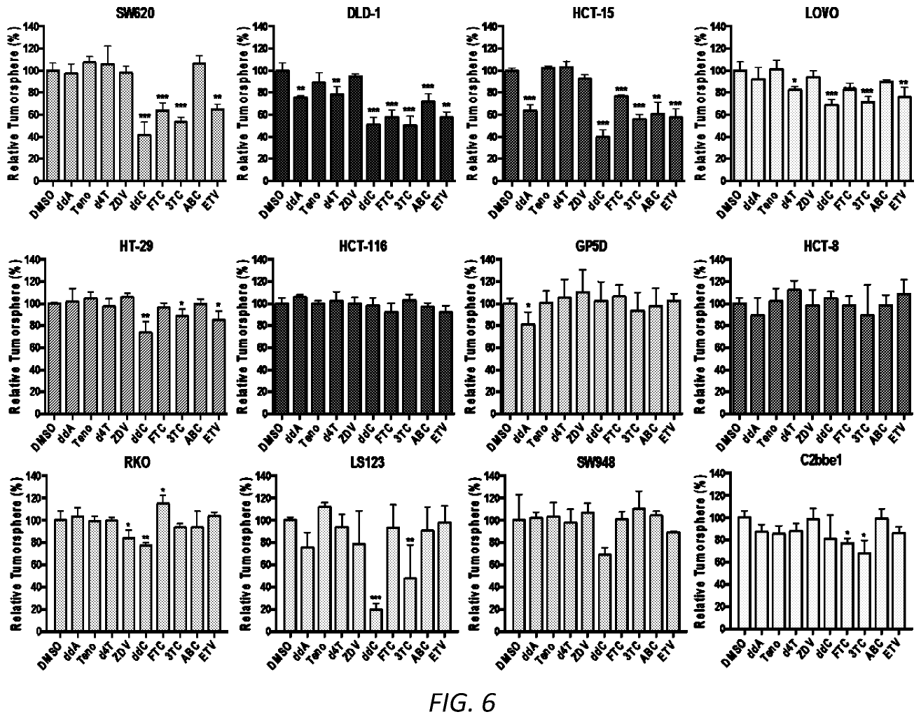

A panel of clinically approved NRTIs was tested on a wide range of colorectal cancer cell lines grown either in 2D adherent conditions, or as 3D tumorspheres. A subset of NRTIs induced cell death in a majority of cell lines grown as 3D tumorspheres. However, these cell lines were resistant to death when grown under 2D adherent conditions (FIGS. 5 and 6). This is consistent with the above-described finding that HSATII RNA is only expressed under 3D growth conditions, and indicates that NRTI induced cell death is a consequence of HSATII RNA accumulation due to reverse transcriptase inhibition. Previous studies on HSATII has provided proof of concept that inhibition of reverse transcription of this satellite repeat RNA can induce death in cancer cells (see Bersani et al. (2015) Proc. Natl. Acad. Sci. U.S.A. 112(49): 15148-53). Thus, inhibiting HSATII reverse transcription by specifically targeting the reverse transcriptase(s) responsible for this activity can be used to treat a wide spectrum of cancers, particularly since HSATII RNA is highly expressed in most epithelial tumors (see Ting et al. (2011) Science 331(6017): 593-6).

### Example 3: HERV-K Reverse Transcriptase is responsible for the reverse transcription of HSATII

The human genome only encodes three proteins with known reverse transcriptase activity: LINE-1 reverse transcriptase (RT), telomerase (TERT), and the human endogenous retrovirus-K reverse transcriptase (HERV-K RT). LINE-1 RT is responsible for reverse transcription of the LINE-1 retrotransposon, and its integration via an endonuclease mediated mechanism. Telomerase is responsible for expansion of the telomeric DNA using the non-coding RNA TERC as a template. HERV-K is the most intact endogenous retrovirus in the human genome, and the only one with a complete and functionally active reverse transcriptase (Berkhout et al. (1999) J. Virol. 73(3): 2365-75). HERV-K reverse transcriptase is known to be expressed in cancer cells. However, the functional consequences of its expression in cancer cells is not understood (Golan et al. (2008) Neoplasia 10(6): 521-33).

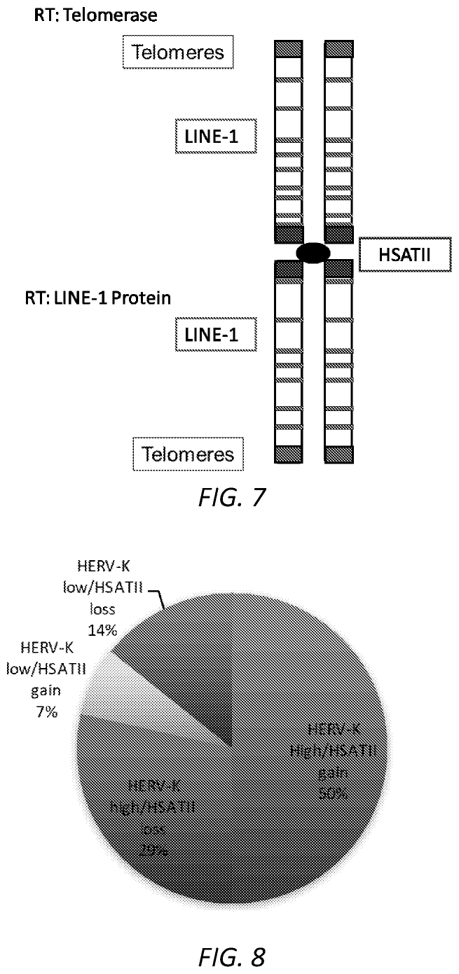

Of the three mammalian RTs, HERV-K was the strongest candidate HSATII reverse transcriptase for multiple reasons. First, there is a 5 base pair similarity (CATTC (SEQ ID NO: 4)) between the HSATII consensus sequence and the repetitive sequence found in the HERV-K 5′ LTR and 3′ LTR. Since viral LTR sequences are critical determinants of reverse transcriptase-mediated genomic integration, it is possible that HSATII reverse transcription and genomic integration is mediated by the HERV-K RT. Second, HSATII and HERV-K are located in the same genomic regions of the chromosomes (i.e., the pericentromeres), whereas LINE-1 is located mostly in euchromatin and is distributed across the genome, and telomeres are present only in the eponymous telomeric regions (FIG. 7). Additionally, pericentromeric expansions of HERV-K have been detected during HIV infection, which are similar to pericentromeric expansions of HSATII in cancer cells (see Zahn et al. (2015) Genome Biol. 16: 74). Finally, genomic data from the Cancer Genome Atlas project (TCGA) suggests that HERV-K expression levels have a high degree of correlation with HSATII copy number gain in subjects having colorectal cancer (P=0.0282; FIG. 8).

Additionally, HERV-K is the only human reverse transcriptase with RNase H activity (Berkhout et al. (1999) J. Virol. 73(3): 2365-75). RNase H causes degradation of the RNA component in a DNA:RNA hybrid post-reverse transcription, and viral reverse transcriptases use this degradation strategy to prevent RNA accumulation, thereby evading detection by innate immune sensors in cells (see Yan and Lieberman (2011) Curr. Opin. Immunol. 23(1): 21-8). The finding that reverse transcriptase inhibition using NRTIs leads to accumulation of HSATII RNA (see Example 1 and FIG. 4) is indicative that the HSATII reverse transcriptase has RNase H activity.

This data supports that HERV-K is the HSATII RT, since it is the only known human RT with RNase H activity.

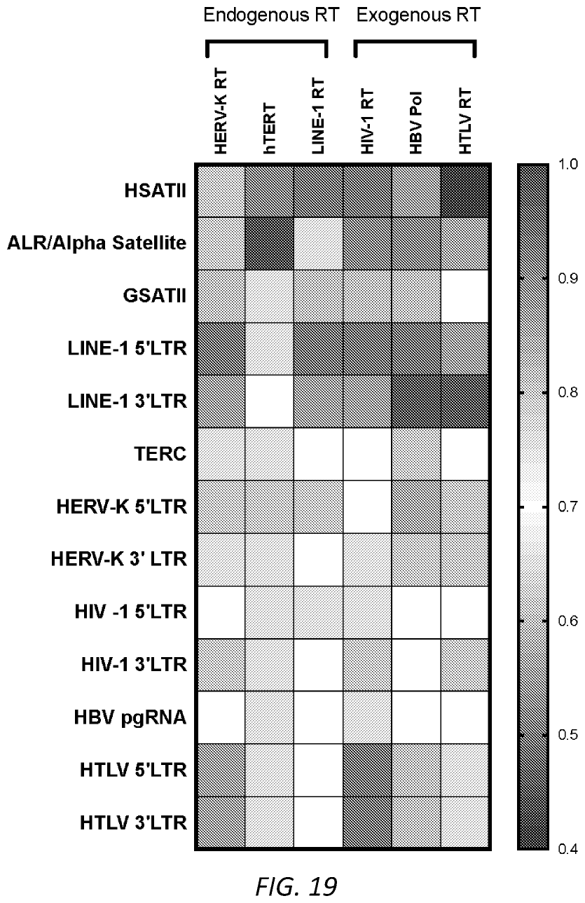

To determine the specificity of HERV-K RT nucleic acid specificity, the following in silico experiment was performed. Briefly, the probability of interaction between each of the reverse transcriptases HERV-K RT (also referred to herein as HERV-K Pol), LINE-1 RT, hTERT, HIV-1 RT, HBV Pol, and HTLV RT, and several RNA repeats of interest was analyzed using the RPIseq software (see Usha et al. (2011) BMC Bioinformatics 12: 489, and Muppirala et al. (2013) J. Comput. Sci. Syst. Biol. 6: 182-7; available at pridb.gdcb.iastate.edu/RPISeq). The RPIseq software output is an RF score from a scale of 0 to 1, with a score >0.5 indicating probability of interaction. As shown in FIG. 19, the satellite sequences HSATII and ALR/Alpha satellite, which are reverse transcribed and highly expressed in cancer cells (see, e.g., Ting et al. (2011) and Bersani et al. (2015)), had a high probability of interaction with HERV-K RT and hTERT as compared to other satellite sequences that are not highly expressed or subject to copy number change in cancer cells (e.g., GSATII). This predictive data indicates that HERV-K RT has a preference/bias for certain RNA repeats, including HSATII.

### Example 4: Inhibition of HSAT II Reverse Transcription Induces Necroptosis in Cancer Cells

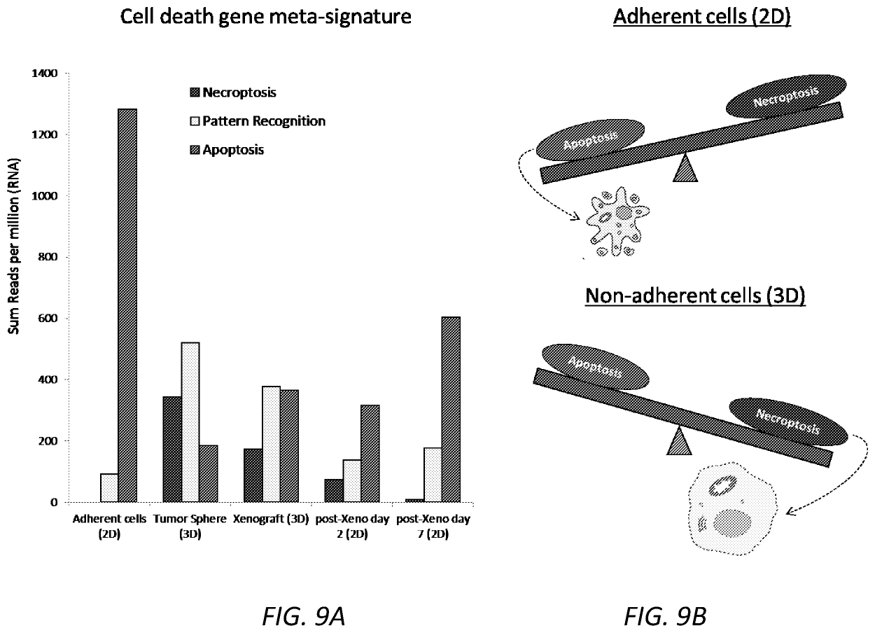

To determine the phenotype induced by inhibiting HSATII reverse transcription the following experiment was performed. RNA sequencing was performed on the SW620 colon cancer cell line grown as either standard 2D culture, 3D tumorspheres, or xenografts. In addition, xenografts were removed and digested for re-culturing in standard 2D culture, which were processed for RNA sequencing 2 and 7 days after plating in culture. Interestingly, cells grown under conditions in which HSATII RNA is expressed (such as tumorspheres or xenografts), expressed higher levels of genes involved in the antiviral innate immune response, such as toll-like receptors, and cytoplasmic pattern recognition receptors (see FIG. 9A). This may be due to a viral mimicry by the HSATII RNA. Further, genes involved in the programmed cell death pathway necroptosis were also enriched.

To further explore this necroptosis phenotype, an experiment was performed whereby the accumulation of HSATII RNA was recreated by introducing artificially synthesized HSATII RNA into adherent cells. Briefly, HCT116 cells were grown on standard 2D adherent cell culture plates. Cells were transfected with ectopic in vitro synthesized HSATII, and treated with either DMSO (vehicle control), or with the NRTIs ddC and d4T either alone or in combination. Cell morphology was analyzed using microscopy. Cell swelling was confirmed as an indication of necroptosis. As shown in FIG. 10, the introduction of artificially synthesized HSATII RNA into adherent cells resulted in cell swelling, an indicator of necroptosis. Moreover, the cellular swelling was enhanced when reverse transcription of the HSATII RNA was blocked using NRTIs. These findings suggest that blocking HSATII RNA reverse transcription induces necroptotic cell death.

To further assess whether necroptosis is responsible for the cellular death and swelling that was observed when HSATII RNA reverse transcription was inhibited, additional experiments were performed whereby the necroptosis pathway proteins RIPK1 and RIPK3 were blocked using the RIPK1 inhibitor necrostatin-1, as well as pharmacological and short hairpin RNAs (shRNAs) specifically targeting RIPK3.

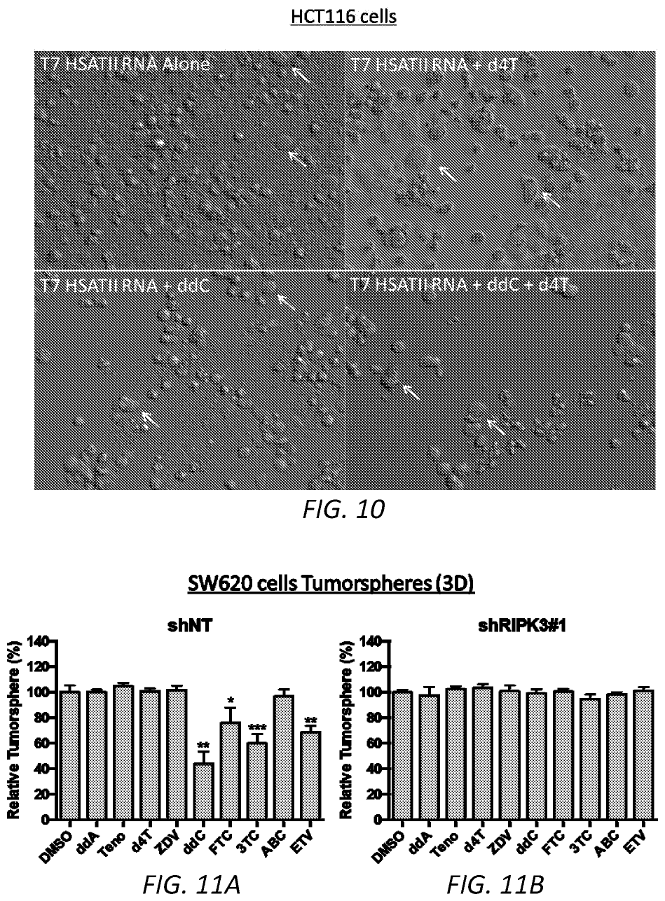

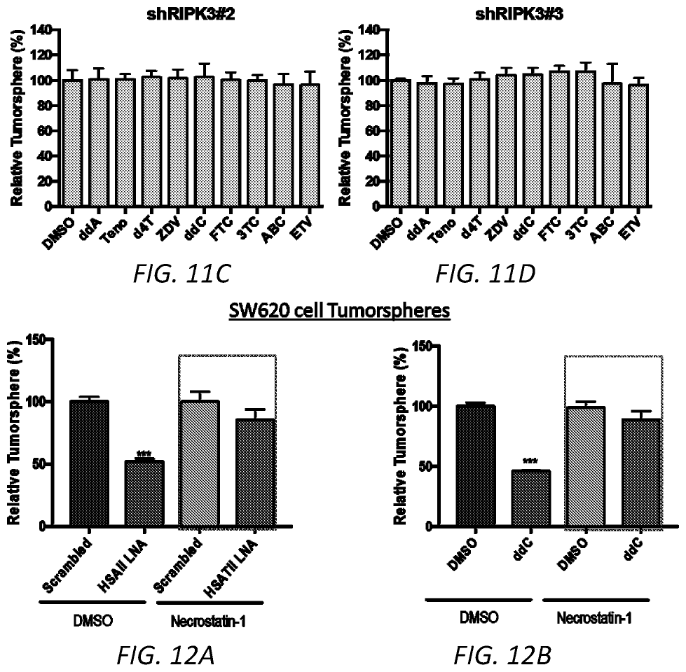

For experiments using shRNAs specifically targeting RIPK3, SW620 cells were transduced with a plko based lentiviral vector encoding either a non-targeting scrambled shRNA (shNT) (see FIG. 11A), or 3 different shRNAs targeting RIPK3 (shRNA for RIPK3 sequences: shRIPK3 #1: 5′-CTGAGAGACAAGGCATGAACT-3′ (SEQ ID NO: 7); shRIPK3 #2: 5′-GTGGCTAAACAAACTGAATCT-3′ (SEQ ID NO:8); shRIPK3 #3: 5′-GCACTCTCGTAATGATGTCAT-3′ (SEQ ID NO: 9) (see FIGS. 11B-11D). Cells were grown as 3D tumorspheres in an ultra-low attachment 96 well plate and treated either with NRTIs or with DMSO. Cellular viability was analyzed using the CellTiter-Glo© Assay (PROMEGA) 5 days post-treatment. As shown in FIG. 11A-11D, blocking of the necroptosis pathway using three different shRNAs specifically-targeting RIPK3 prevented the cell death induced by the NRTIs ddC, 3TC, ABC, and ETV.

For experiments using the RIPK1 inhibitor necrostatin-1, SW620 cells were either transfected either with scrambled non-targeting LNA (5′-AACACGTCTATACGC-3′ (SEQ ID NO: 5)) or an LNA targeting HSATII (5-+A*+T*+G*+G*A*A*T*C*A*T*C*A*T*+C*+G*+A*+A-3′ (SEQ ID NO: 10; *=phosphorothioate bond; +=locked nucleic acid base)) using Lipofectamine© and treated with DMSO (control) or 10 μM necrostatin-1 (FIG. 12A), or treated either with DMSO or 5 μM ddC in the presence or absence of 10 μM necrostatin-1 (FIG. 12B). Cells were seeded and grown as 3D tumorspheres in an ultra low attachment 96 well plate 1 day after transfection. As shown in FIGS. 12A and 12B, blocking of RIPK1 with necrostatin-1 prevented tumor cell death induced by treatment of the cells with either an HSATII-specific LNA or the NRTI, 2′,3′-dideoxycytidine (ddC). These results confirm that HSATII reverse transcription inhibition induces necroptotic cell death in cancer cells.

The induction of necroptosis using an inhibitor of HSATII reverse transcription may be particularly advantageous in the treatment of cancer. Most conventional chemotherapy drugs and targeted therapy compounds induce cell death through apoptosis instead of necroptosis. The induction of cancer cell death via apoptosis does not stimulate the immune response. However, necroptotic cell death is immunogenic. Therefore, without wishing to be bound by any particular theory, the induction of necroptosis may be particularly advantageous as it may result in the added benefit of inducing anti-tumor immunity.

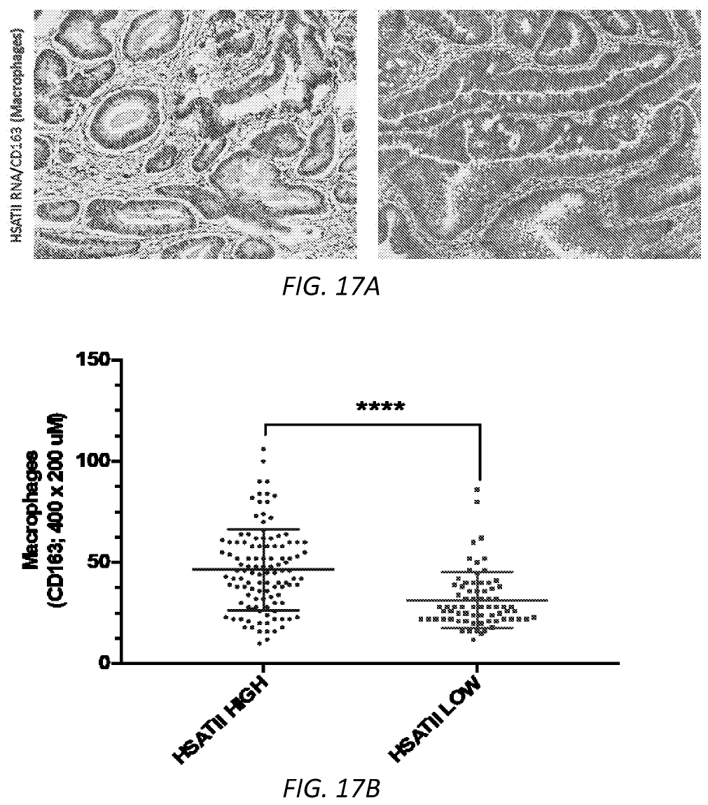

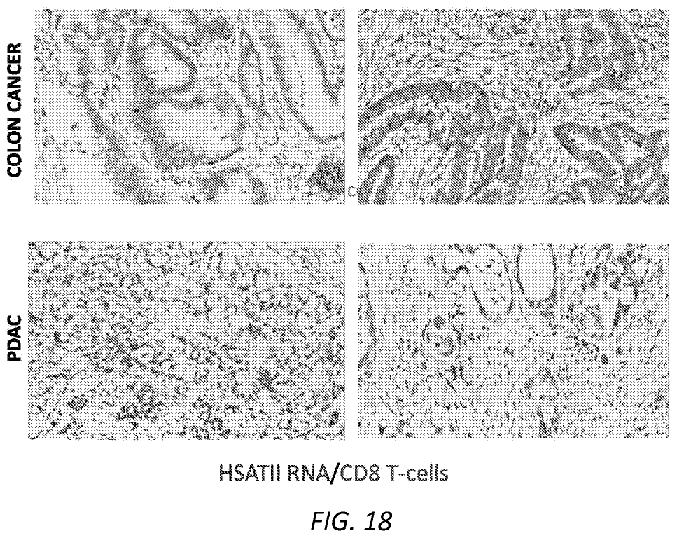

To determine the potential role of HSATII RNA levels in anti-tumor immunity, the following experiments were performed. Briefly, dual color RNA-ISH for HSATII (red) and immunohistochemistry for the macrophage marker CD163 or CD8 (brown) was conducted on human tissue microarrays of colon and pancreatic (PDAC) cancers. Tumors were scored by a pathologist, and classified either as HSATII high or HSATII low based on HSATII RNA levels. Number of CD163⁺ macrophages were counted in tumor, and plotted according to tumor HSATII status. The data suggests a correlation between antitumor immunity and HSATII, since HSATII RNA levels correlated with the presence of intratumoral macrophages, and anti-correlates with the presence of CD8⁺ T-cells (FIGS. 17A, 17B, and 18). Hence, targeting the HSATII reverse transcriptase may also provide a novel immunotherapeutic approach for the treatment of cancer.

### Example 5: Combination Therapy with an NRTI and a DNA Demethylating Agent Enhances Cell Death in Cancer Cells Resistant to NRTI Treatment Alone

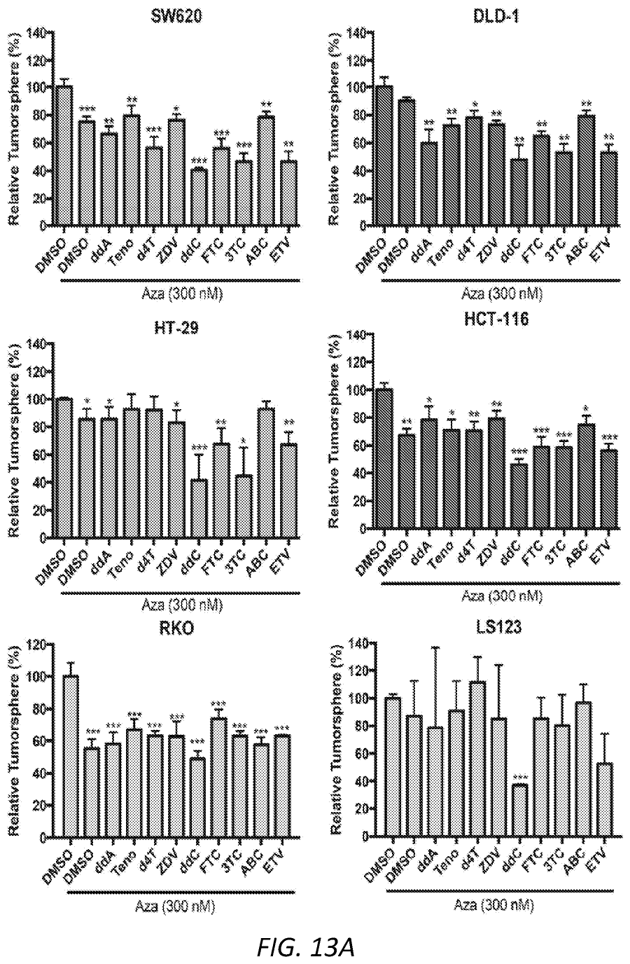

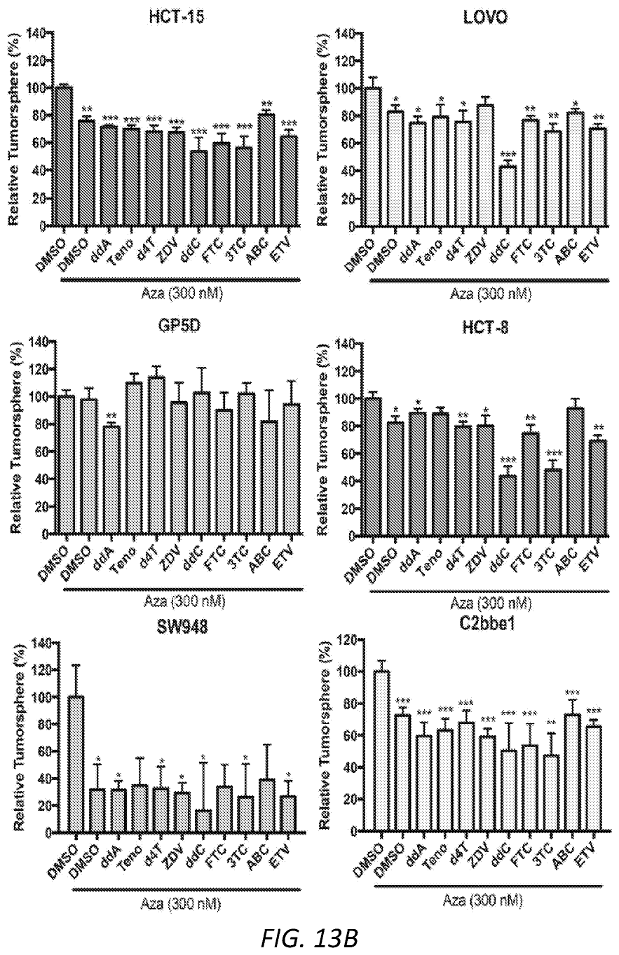

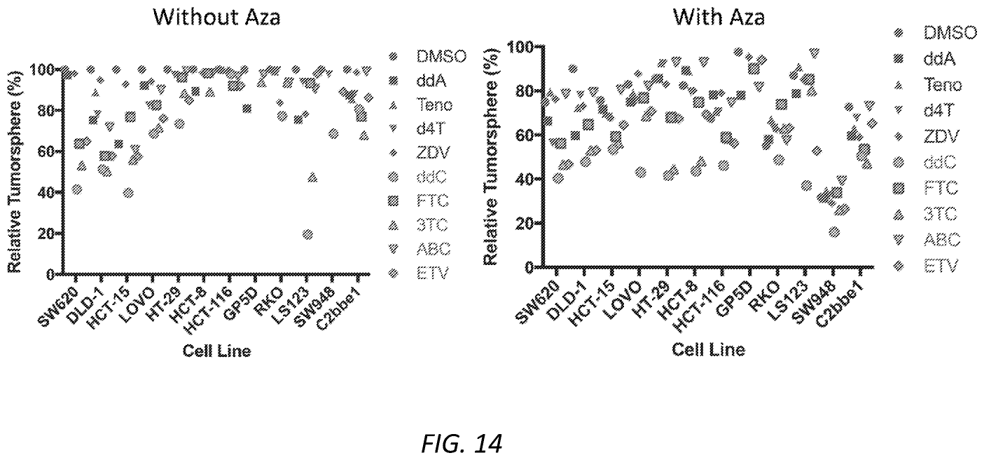

As described above, some cancer cell lines appear to be refractory to treatment with an NRTI (e.g., the Microsatellite Stable (MSS) colorectal cancer cell lines SW620 and DLD-1, see Example 1). To determine whether activation of satellite transcription using the DNA hypomethylating agent 5′-azacytidine (Aza) could render MSS colorectal cancer cell lines sensitive to NRTI treatment, the following experiment was performed. Briefly, human colorectal cancer cell lines were grown as 3D tumorspheres in ultra-low attachment 96 well plates and treated either with DMSO or with 5 μM of NRTI in the presence of 300 nM Aza. A control sample (DMSO) without Aza or NRTI was also included. Cell viability was analyzed using the CellTiter-Glo® Assay (PROMEGA) 5 days after treatment initiation. Percent cell viability was calculated by normalizing luminescence to the control sample (DMSO without AZA (FIG. 13). A summary of the data from FIG. 6 (no Aza) and FIGS. 13A and 13B (with Aza) is shown in FIG. 14, which highlights the increased efficacy of the combination of AZA and NRTI across cancer cell lines. Surprisingly, reverse transcriptase inhibition using an NRTI in combination with 5′-azacytidine enhanced cell death in colorectal cancer cell lines. Cell lines that were previously refractory to NRTIs became sensitive when treated with both the NRTI and 5′-azacytidine (FIGS. 13A, 13B, and 14).

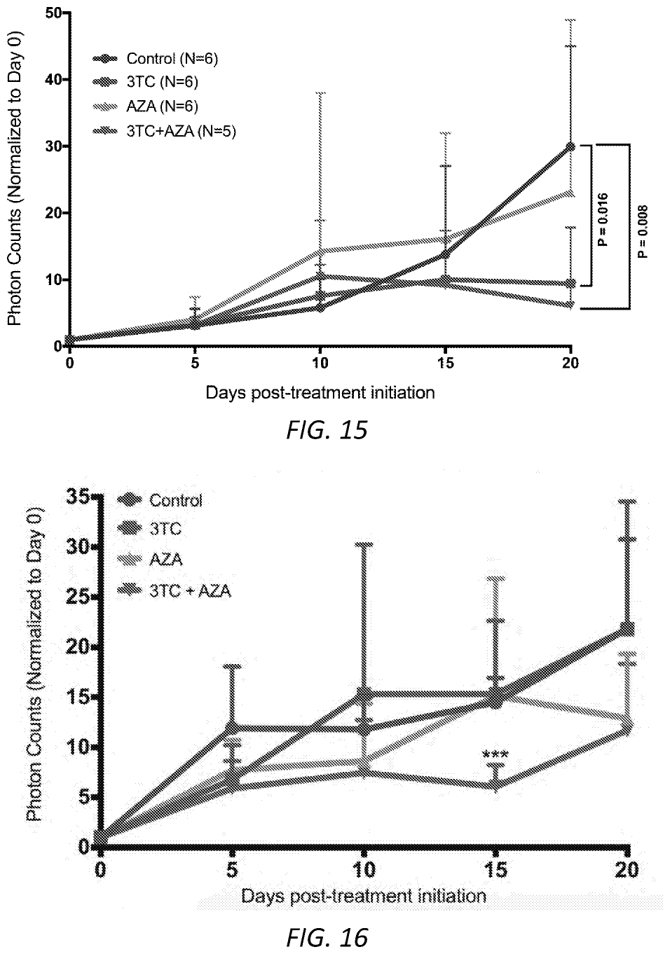

To determine if this in vitro effect could be replicated in vivo, xenograft colorectal cancer mouse models were used. Briefly, SW620 (500,000 cells in Matrigel©) or HCT116 (1,000,000 cells in Matrigel©) colorectal cancer cells transduced with a luciferase gene were implanted subcutaneously in the right flank of athymic nude mice. Tumors were allowed to grow for 12 days (SW620) or 7 days (HCT116). IVIS imaging was conducted after intraperitoneal injection with 150 μl luciferin (300 mg/kg in PBS) at treatment day 0. Relative photon counts were used as representation of tumor size. Mice were randomized into 4 cohorts as indicated: PBS (Control), 3TC (50 mg/kg), Aza (0.75 mg/kg), and 3TC (50 mg/kg)+Aza (0.75 mg/kg) (see FIGS. 15 and 16). Drugs or PBS were administered 3 times per week via intraperitoneal injection. Tumor growth was measured by IVIS imaging every 5 days. As shown in FIGS. 15 and 16, combination therapy with the NRTI lamivudine (3TC) and 5′-azacytidine induced a reduction in xenograft tumors of the MSI cell line HCT 116, which is refractory to NRTIs. Accordingly, combination therapy of an NRTI and a DNA hypomethylating agent, such as 5′-azacytidine, is useful to treat subjects having tumors that are resistant to treatment with a HERV-K reverse transcriptase inhibitor alone.

### Example 6: Inhibition of HERV-K Expression Shows Inhibition of Cancer Cell Growth in 3D Cell Culture System

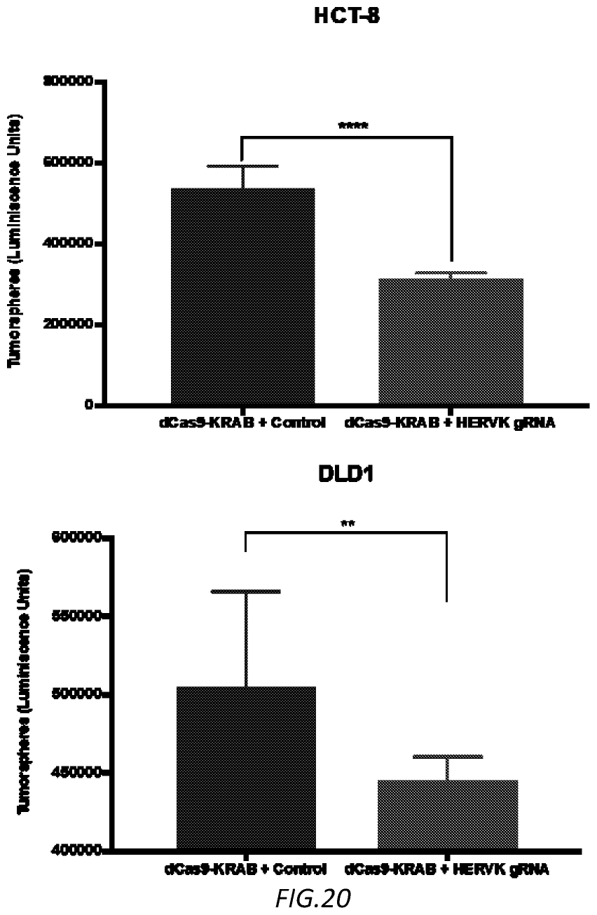

A CRISPR KRAB transcriptional repressor system was used to demonstrate that inhibition of HERV-K significantly reduced colon cancer cell line growth in 3D tumor spheres containing two separate cancer cell lines, HCT-8 and DLD1 (see FIG. 20). Significant reduction in tumorspheres was observed when in CRISPR KRAB transcriptional repressor system including HERVK gRNA was used, as compared to control gRNA. P-value by Welch's t-test. ** p=0.0063. **** p<0.0001 This finding is consistent with our other data pointing towards HERV-K as the endogenous reverse transcriptase (see Example 3). This data also demonstrates that suppression of HERV-K using a CRISPR nuclease systems (e.g., Cpf1, CRISPR/Cas9, CRISPR/Cas13) is a valid approach for the treatment of cancer.

### Example 7: TP53 Linked with Regulation of Repeat RNA Expression and Differential Sensitivity to Repeatome Drugs

Repetitive elements constitute >50% of the human genome and their aberrant expression in cancers suggests a functional importance of the “Repeatome” that can be exploited as a therapeutic vulnerability. The Repeatome includes the expression of a variety repeat RNAs including LINE-1 retrotransposons, human endogenous retroviruses (HERV), and satellite repeats. These repeat RNAs have distinct expression patterns across cancers, and are associated with unique epigenetic and immunologic features. Modulation of repeat RNA expression through genetic and epigenetic modifiers can engage the innate and adaptive immune response. Many of these repeat RNAs are known to replicate through a reverse transcriptase dependent mechanism including LINE-1, HERV, and satellite repeats. The inhibition of the reverse transcriptional activity of each of these repeat RNA classes has been demonstrated with NRTIs, a class of agents commonly used in HIV. However, the therapeutic potential of NRTIs and other Repeatome modulating agents in cancer remains to be fully characterized and translated into the clinic.

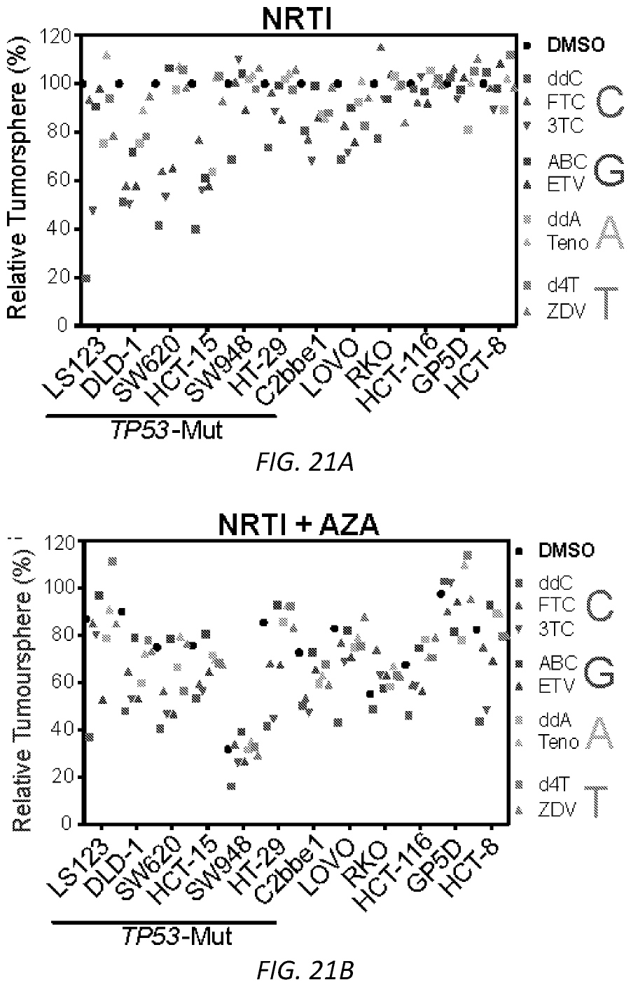

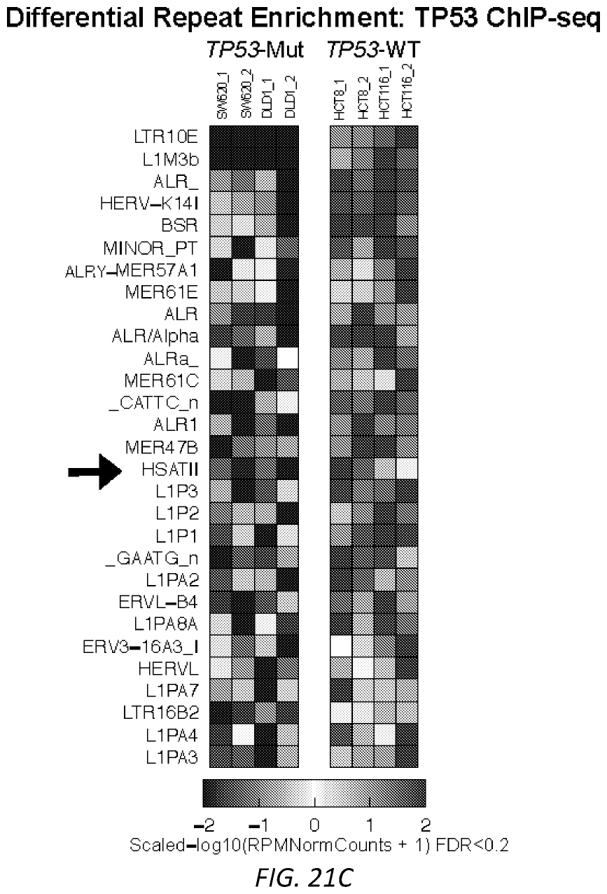

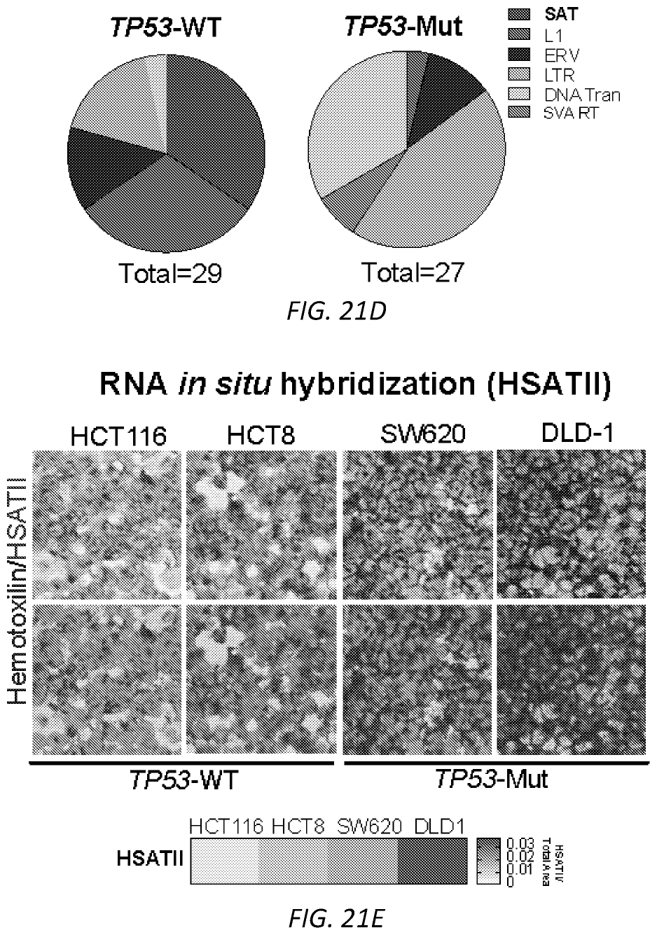

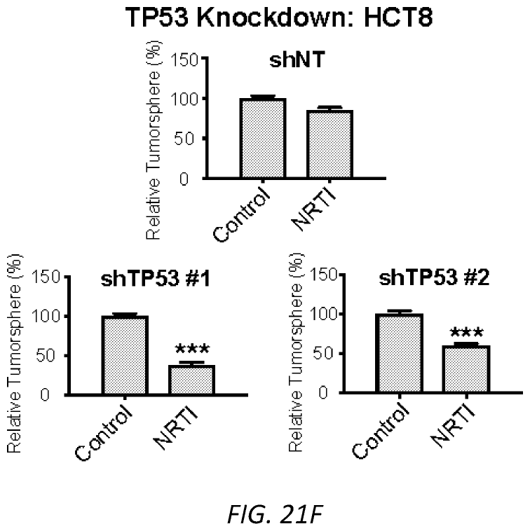

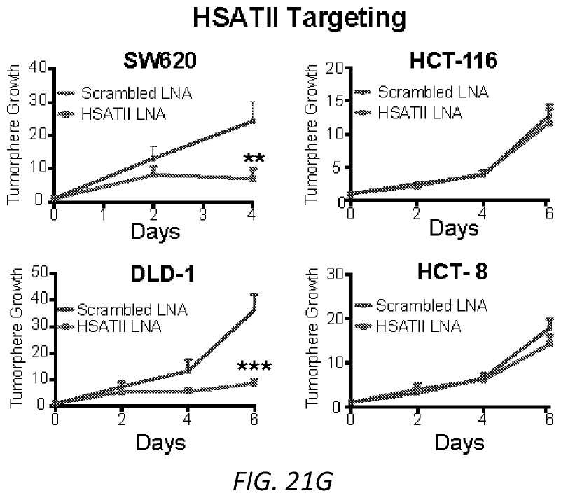

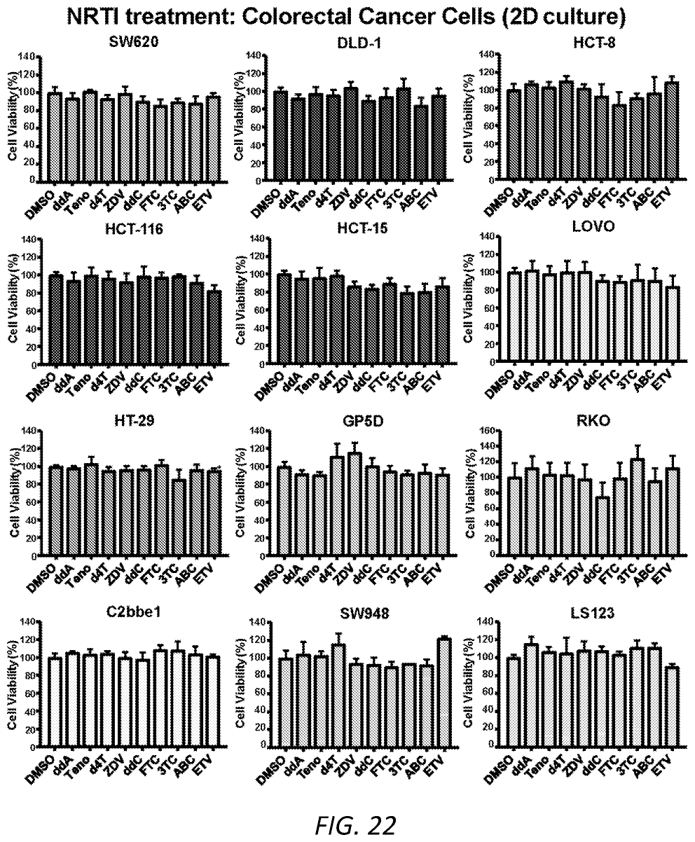

To determine the generalizability of NRTI anti-neoplastic effects, a panel of 9 NRTIs encompassing analogues for each of the nucleosides on a set of 12 colorectal cancer (CRC) cell lines was tested. As shown in FIGS. 21A and 22, although none of the NRTIs had any effect in standard 2D adherent culture, there were significant cytotoxic effects on these same lines grown in 3D tumoursphere cultures. This suggests that there is a lack of repeat RNA expression in 2D culture that can be induced in 3D culture. Interestingly, as shown in FIG. 21A, only the C and G nucleoside analogues had consistent cytotoxic effects on cancer cell lines, which indicated that RT inhibition of specific RNA sequences with high C and G content were important for response. This differential response is supported by the demonstration of high CpG motif repeats having enhanced viral pattern recognition receptor (PRR) response as was seen with the HSATII satellite repeat, a particular repeat that has been identified as being highly specific for cancers compared to normal tissues.

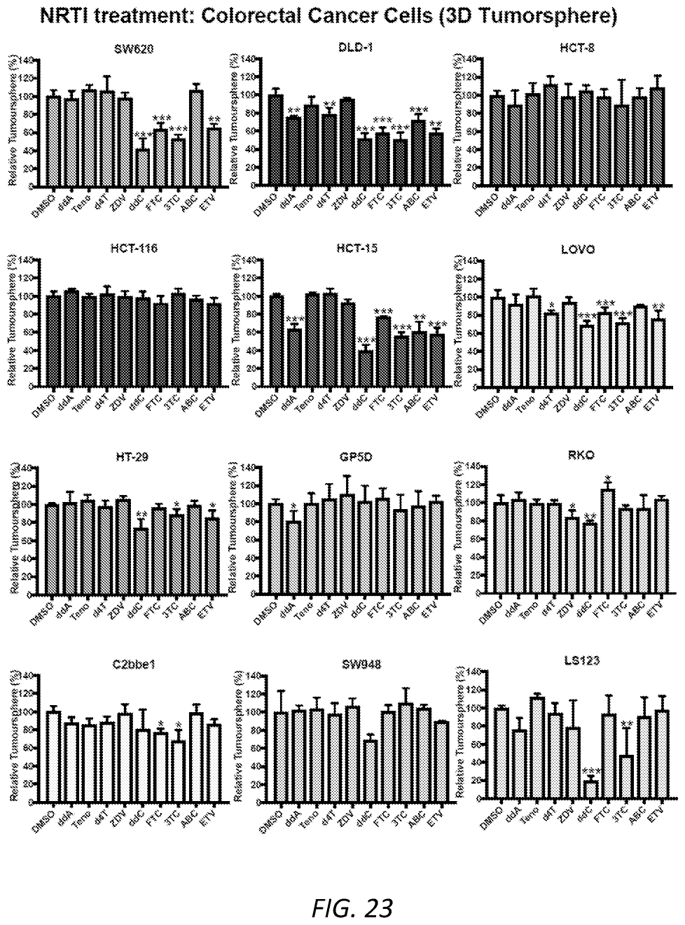

Next, the possibility of combining Repeatome modulating agents with the DNA hypomethylating agent 5-azacitidine (AZA) was explored in the panel of NRTIs in our CRC cell lines. AZA has been shown to derepress a wide range of repeat RNAs. As shown in FIGS. 21B and 23, this combination demonstrated broad cytotoxic activity in all CRC cell lines, which indicated intrinsic differences between cancer cell lines that predispose sensitivity to one type of Repeatome targeting agents over another. This multi-targeted approach to disrupting the Repeatome is analogous to multidrug therapies for retroviral infections.

Evaluation of potential genomic determinants of drug response noted differential NRTI sensitivity of TP53-mutant (TP53-Mut: SW620, LS123, DLD1, HCT-15, HT-29, SW948, C2bbe1) compared to TP53-wildtype (TP53-WT: LOVO, HCT-116, HCT-8, RKO, GP5D) cell lines, as shown in FIG. 21A. TP53 has emerged as an important epigenetic regulator with direct linkage to repeat RNA expression. To determine if TP53 mutation leads to differences in direct interactions with repeat DNA, TP53 immunoprecipitation (IP) followed by DNA sequencing in TP53-Mut (SW620, DLD1) and TP53-WT (HCT-116, HCT-8) cell lines was performed. As shown in FIGS. 21C and 21D, Differential enrichment analysis of TP53 bound repeat elements (FDR<0.2) demonstrated markedly different proportions of specific repeat classes with notable higher satellite (SAT 34% of repeats) and LINE1 (L1 31% of repeats) elements in TP53-WT compared to TP53-Mut cell lines. Both TP53-mutant cell lines have DNA binding domain mutations (SW620-R273H; DLD1-S241F), which suggests that loss of function of DNA binding is associated with diminished TP53 interaction with these repeat DNA sequences. These same SAT and L1 repeats were previously shown to be highly expressed in a broad set of cancers, with the cancer specific HSATII satellite repeat being enriched in our TP53 IP sequencing analysis. To evaluate if HSATII repeat RNA expression was linked with these differences in TP53 binding, quantitative RNA in situ hybridization (RNA-ISH) was performed. As shown in FIG. 21E, there was higher HSATII repeat expression in TP53-Mut (SW620, DLD1) compared to TP53-WT (HCT-8, HCT-116) cell lines. Collectively, these data (1) support linking expression of L1 repeats with TP53-Mut cell lines as well as in other model organism systems, and (2) provide evidence of TP53 direct suppression of both SAT and L1 repeats in colon cancer.

Based on the above data, the loss of TP53 suppression of repeat RNAs could trigger sensitivity to NRTIs. To test this hypothesis, HCT8 cells were treated with shRNA to target wildtype TP53. Indeed, as shown in FIG. 21F, shRNA mediated suppression of wildtype TP53 in HCT8 cells led to sensitivity to NRTI. The loss of DNA binding to repeat sequences in TP53-Mut cell lines and the induction of NRTI sensitivity with shRNA mediated suppression of wildtype TP53 in HCT-8 cells would point towards a loss of function effect of TP53 on repeat expression.

Finally, given TP53 suppressed specific repeats, it was questioned whether therapeutically targeting these repeats with locked nucleic acids (LNAs) would have similar effects in cell lines. Since there was consistent loss of HSATII binding by mutant TP53 (FIG. 21C) and the associated higher HSATII RNA expression (FIG. 21E), HSATII satellite repeats were targeted. Indeed, as shown in FIG. 21G, there was specific inhibition of TP53-Mut compared to WT cell line growth with HSATII LNAs compared to scrambled LNA controls. Collectively, these data show a unique relationship of TP53 in suppressing SAT and L1 repeats that lead to specific sensitivities in mutant TP53 cell lines to reverse transcriptase inhibitors and sequence specific LNAs.

### Example 8: Repeatome Modulation Correlated with Chromatin Factors and Associated with Necroptotic Cell Death

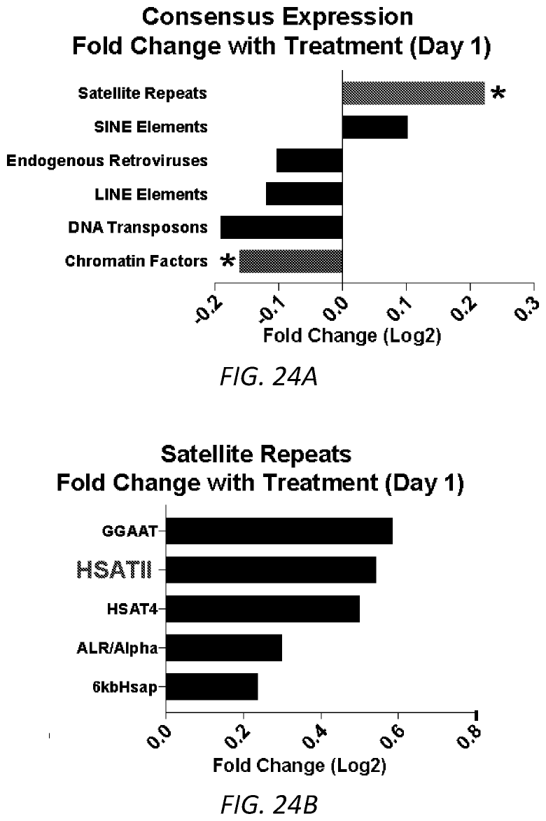

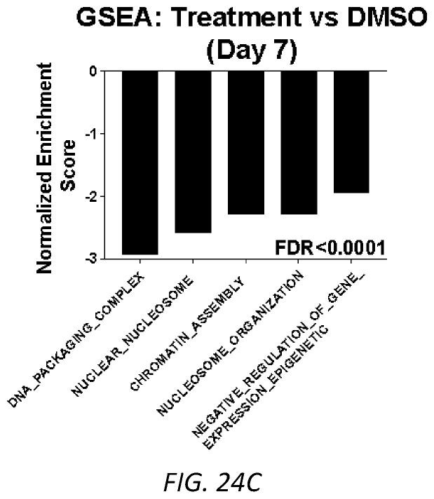

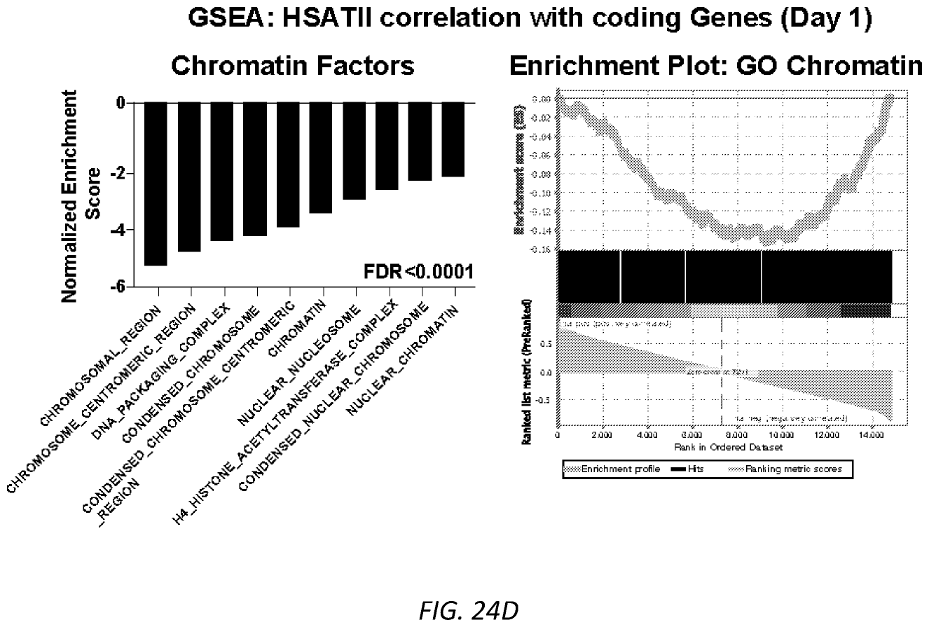

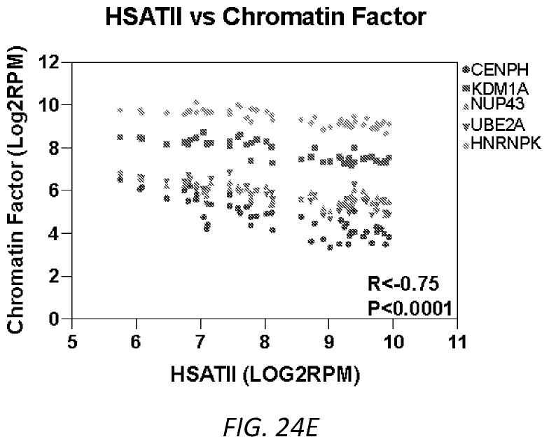

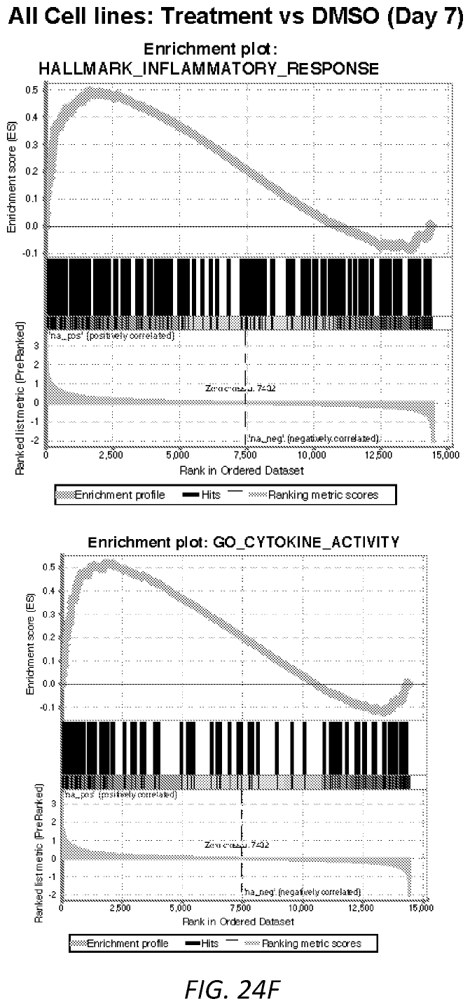

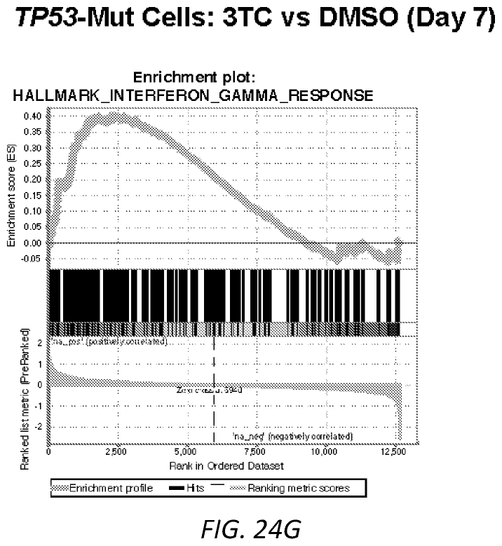

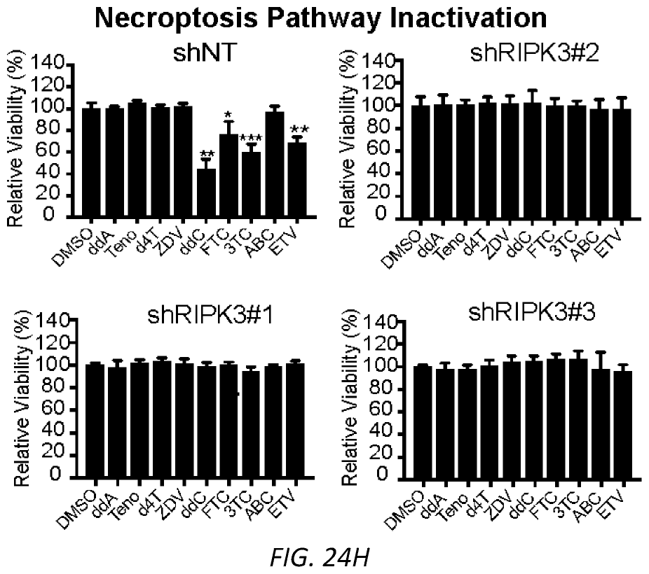

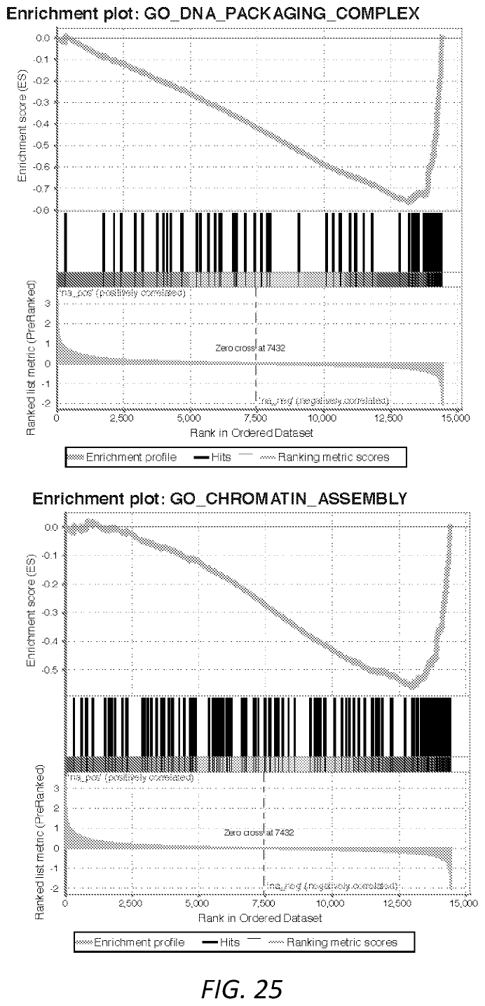

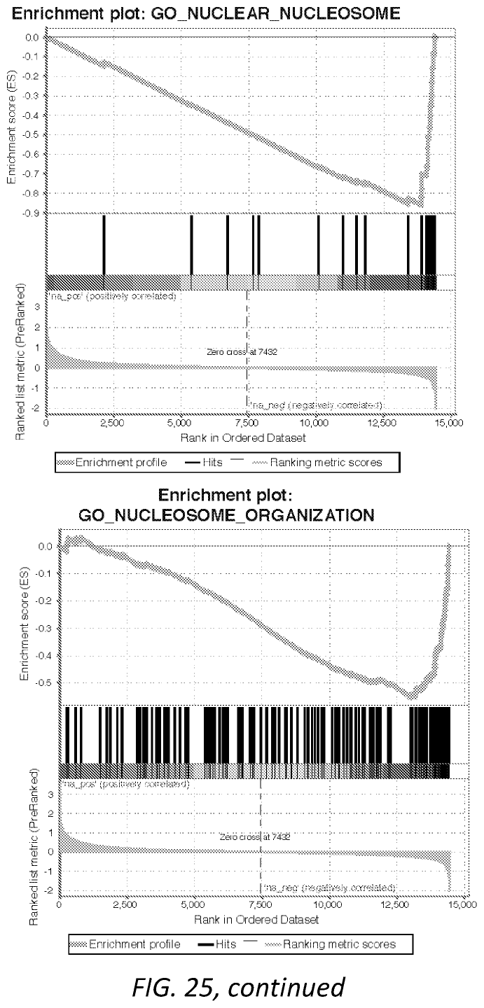

Next, the mechanism of Repeatome targeted cytotoxicity was examined by performing RNA-seq of CRC cell lines 1 day after treatment with the NRTI lamivudine (3TC), AZA, the combination, or DMSO control (triplicate RNA-seq for each condition). Analysis of consensus expression of repeat elements differentially expressed by these Repeatome disrupting agents demonstrated significant changes (FDR<0.05) in SAT repeats compared to other repeats (FIG. 24A). Analysis of specific satellite repeats elevated with Repeatome drugs identified GGAAT, HSATII, HSAT4, ALR, and 6kbHsap with highest fold induction (FIG. 24B), where both HSATII and ALR were also seen in the TP53-IP sequencing experiments (FIG. 21C). Gene set enrichment analysis (GSEA) of coding genes interestingly demonstrated negative enrichment of multiple chromatin factors in treated vs DMSO control cell lines (FIGS. 24C and 25), which was also seen when performing consensus expression of a comprehensive set of chromatin factors (FIG. 24A). Given the apparent inverse relationship of chromatin factors and SAT repeats, we evaluated if there was a correlation of expression between these factors and not simply association. Linear correlation analysis of HSATII repeat expression with all coding genes across all samples and genes were ranked using the Pearson R coefficient (FIG. 24D). GSEA of HSATII ranked correlated genes showed a striking enrichment of chromatin factors using a Pearson R cutoff of <−0.75 (FIG. 24E). The most highly anti-correlated chromatin factors included the centromeric protein CENPH, the lysine specific demethylase KDMIA, the nucleoporin protein NUP43, the E2 ubiquitin conjugating enzyme UBE2A, and the heterogeneous ribonucleotide protein HNRNPK. Notably, KDM1A otherwise known as LSD1 has been shown to be important in repeat RNA suppression.

The early induction of repeat RNAs by 3TC and AZA suggested that there might be an inflammatory innate immune response. Indeed, analysis of RNA-seq of CRC cell lines 7 days post-treatment of all drug conditions versus DMSO (triplicate RNA-seq for each condition; FIG. 24F) revealed significant enrichment of HALLMARK_INFLAMMATORY_RESPONSE (normalized enrichment score (NES)=1.67; FDR=0.19) and GO: Cytokine Activity (NES=1.68; FDR=0.18). Specific analysis of TP53-mutant cell lines demonstrated significant enrichment for HALLMARK_INTERFERON_GAMMA_RESPONSE (NES 1.625; FDR 0.25) in 3TC versus DMSO control cell lines (FIG. 24G). Collectively, these analyses are consistent with NRTI and AZA as being activators of the innate immune response due to induction of immuno-stimulatory repeat RNAs.

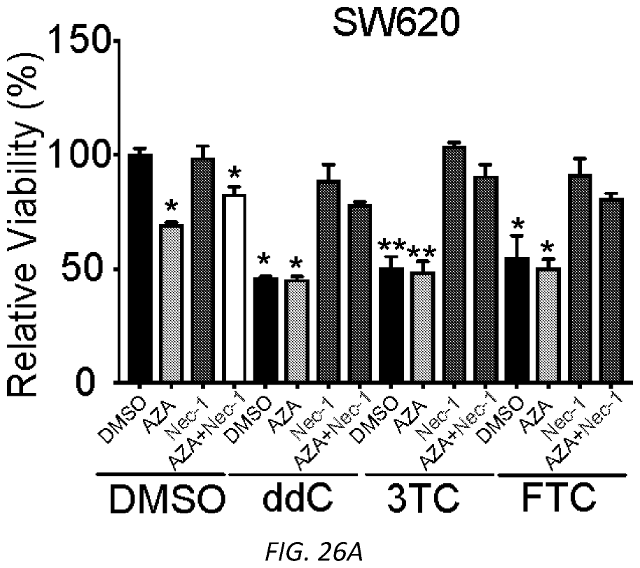

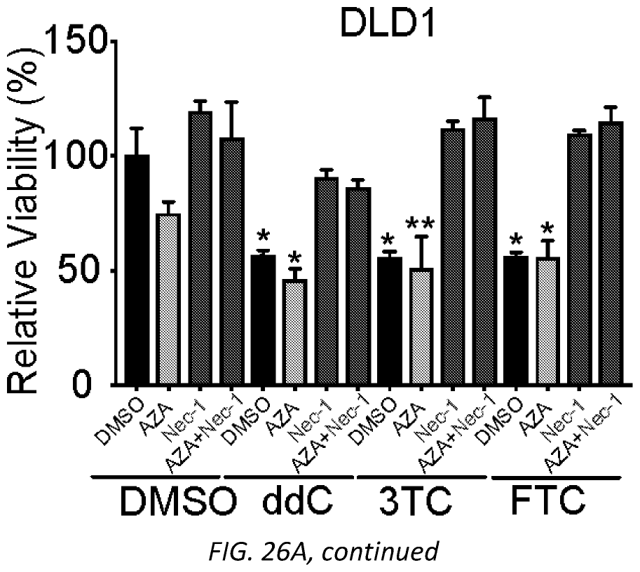

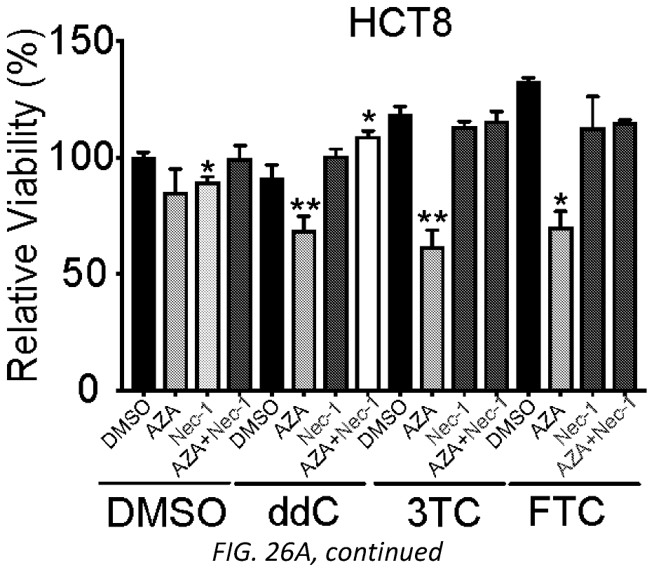

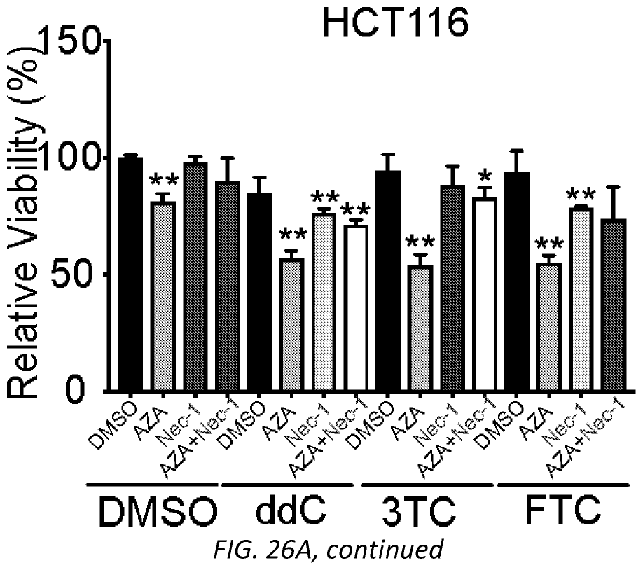

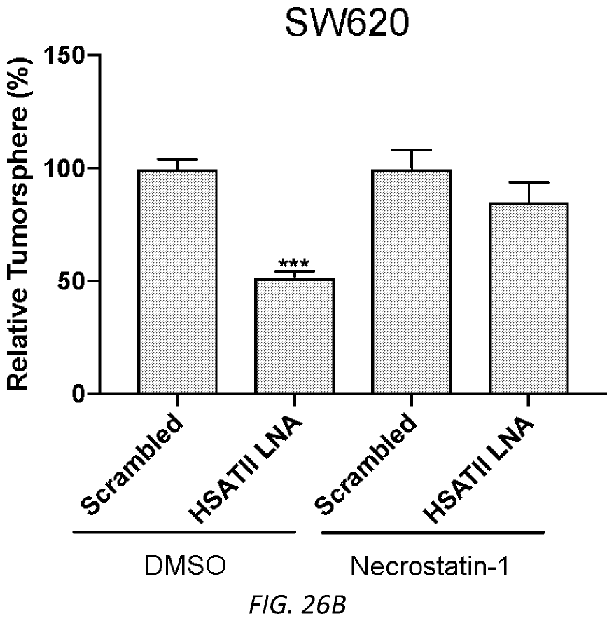

Next, the aetiology of cancer cell toxicity induced by repeat RNA stress was examined. In particular, given the relationship with cell-autonomous cytokine release and indicating that mutant TP53 primes epithelial cells to necroptosis22, necroptosis was examined. Indeed, inhibition of necroptosis effectors RIPK1 by the small molecule inhibitor Necrostatin-1 (FIGS. 26A-26B) or RIPK3 by shRNA (FIG. 24H) were each sufficient to rescue cell lines from NRTI and AZA mediated toxicity.

### Example 9: Repeatome Targeting Drugs with In Vivo Efficacy and Complementary Effects with Cytotoxic Therapies

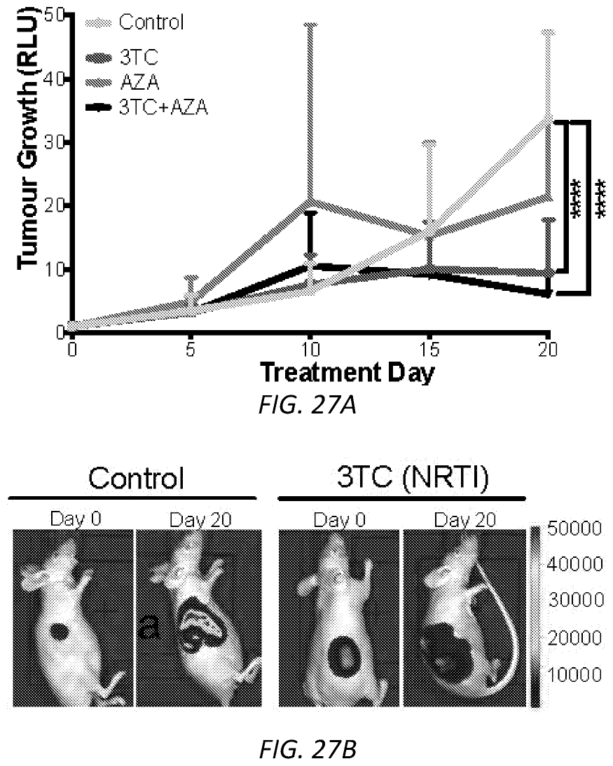

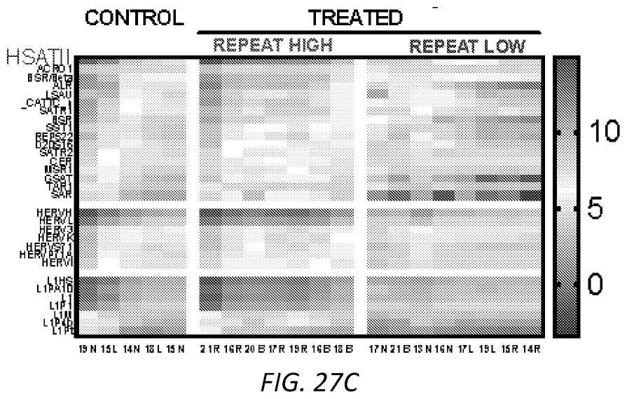

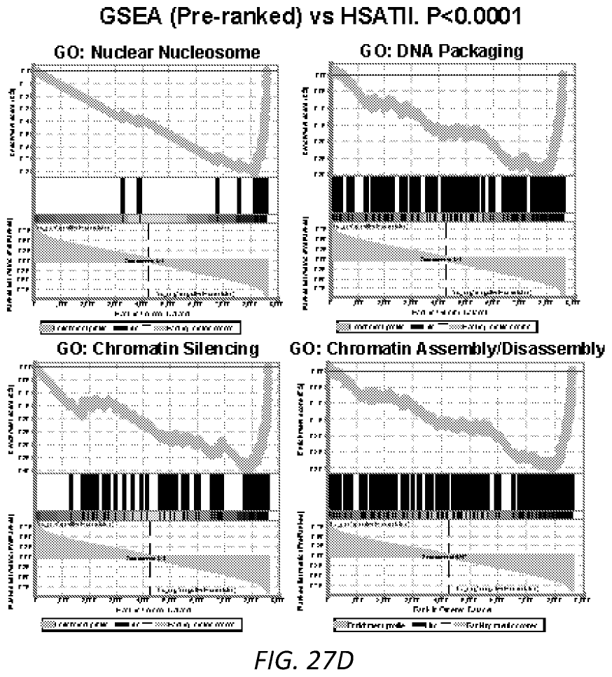

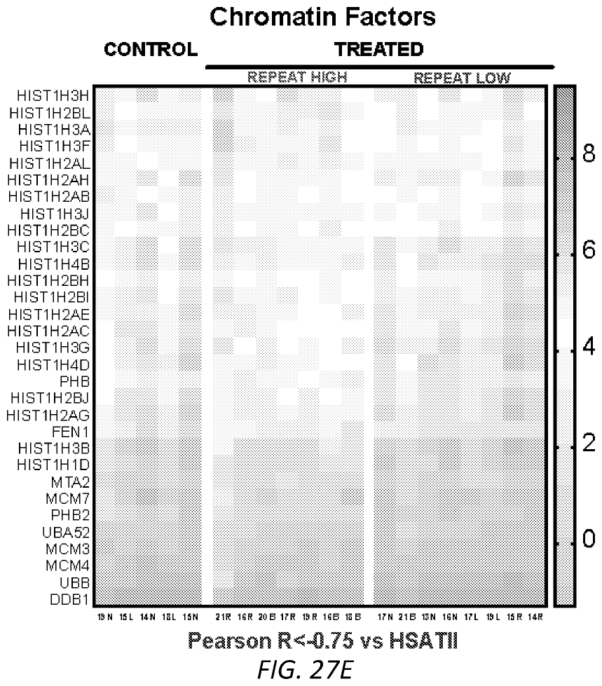

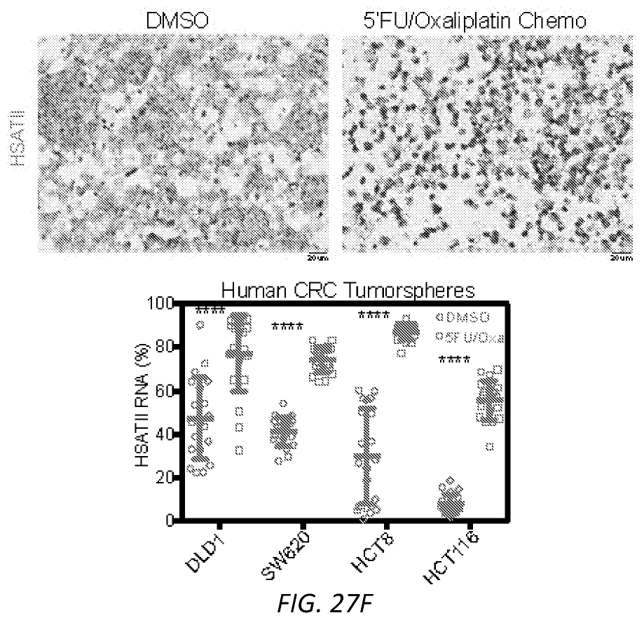

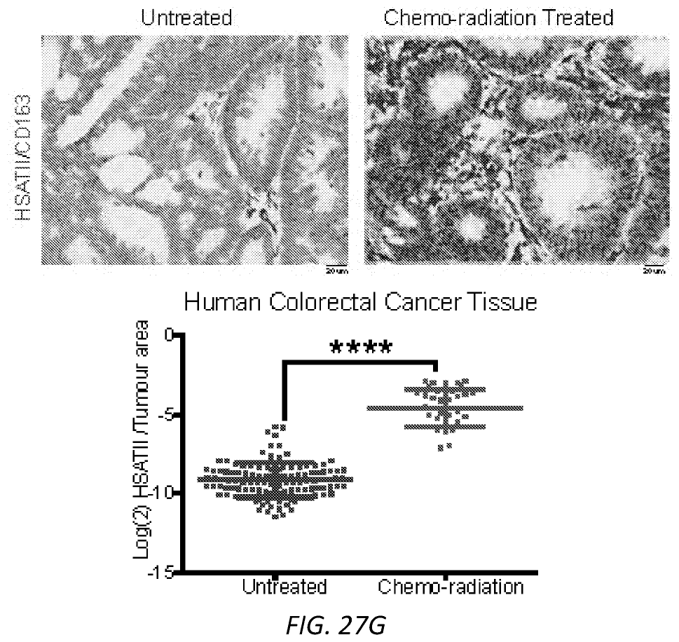

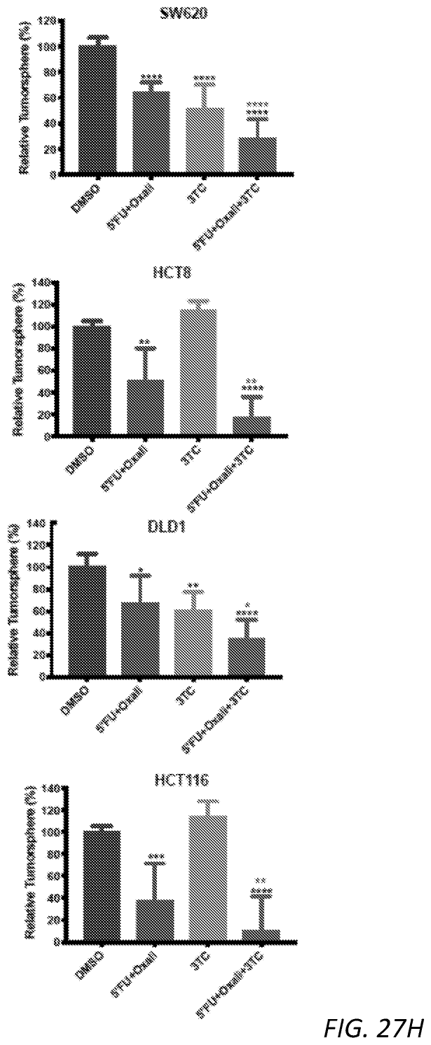

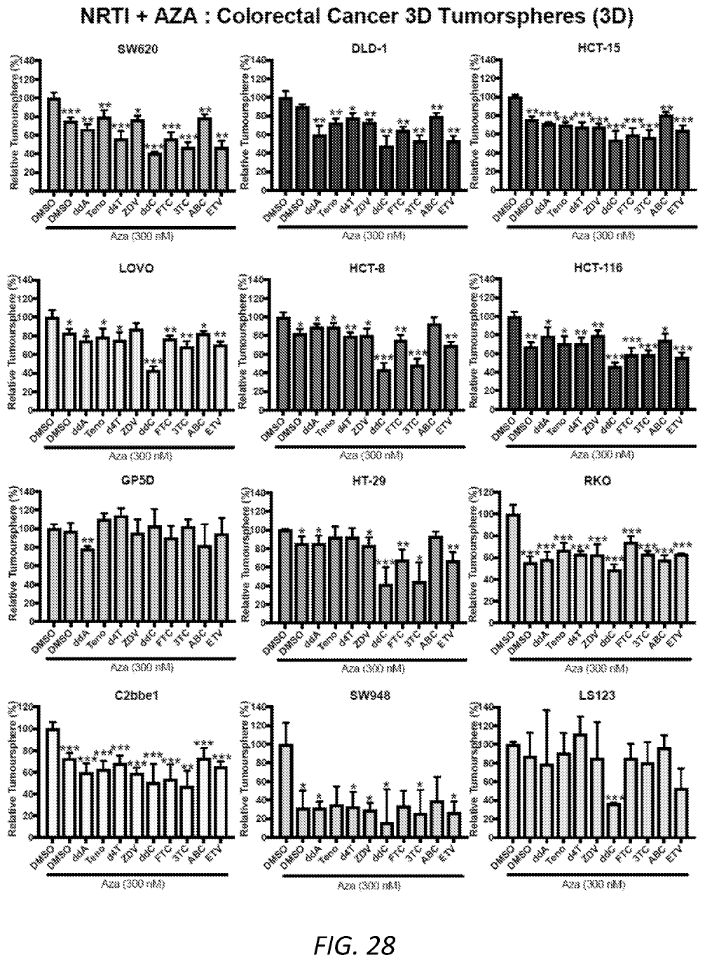

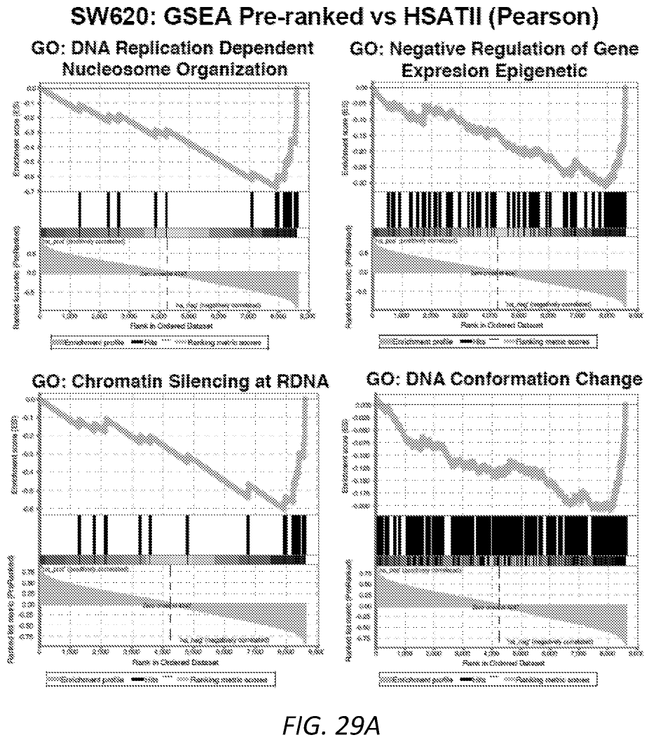

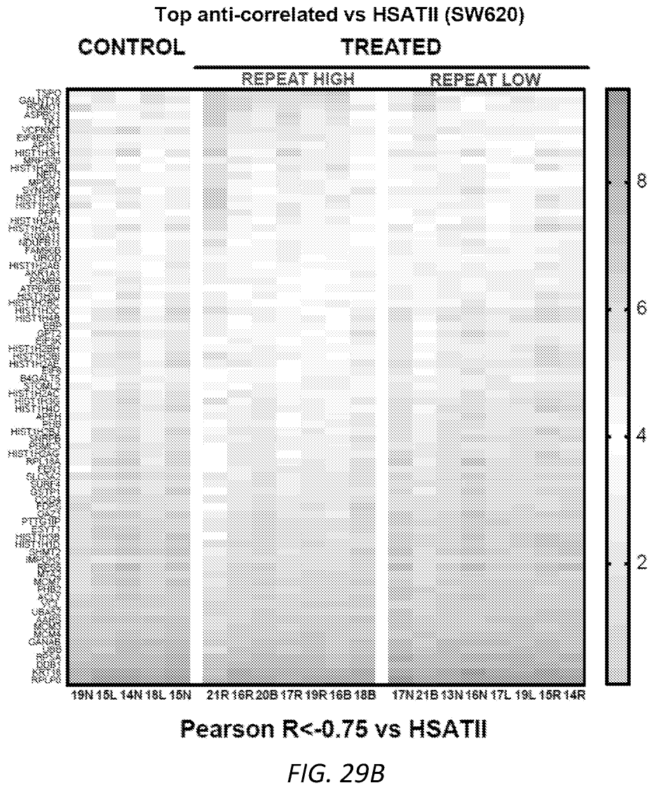

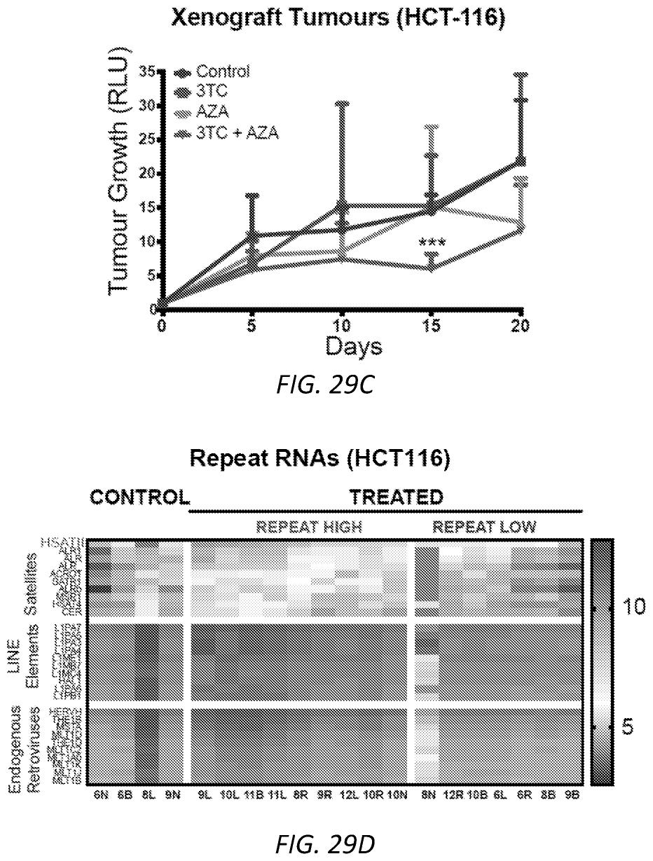

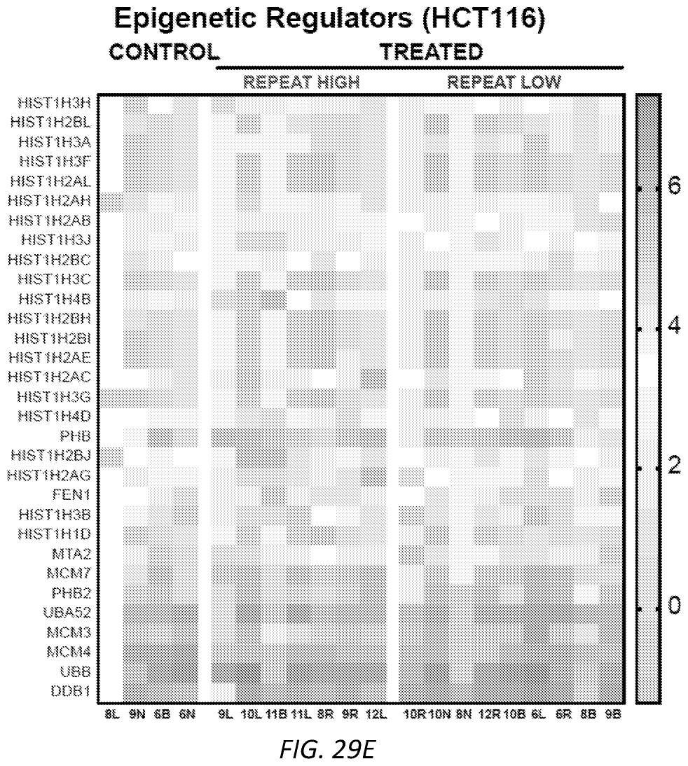

The in vitro findings of Examples 7 and 8 were extended to xenograft tumours using a cell line with baseline high (SW620) and low (HCT-116) repeat RNA expression. Mice had subcutaneous tumours generated with luciferase-transduced cell lines over 2 weeks followed by three times weekly dosing of 3TC at 50 mg/kg and/or AZA at 0.75 mg/kg administered by intraperitoneal injection. Tumour growth was monitored by in vivo luminescence (IVIS) imaging, and the experiment was completed at 20 days due to maximal allowable tumour size in the control group. The repeat-high xenograft SW620 demonstrated significant response to 3TC alone (ANOVA p value<0.0001) and improved response with combination 3TC+AZA (ANOVA p value<0.0001) compared to vehicle control treated tumours (FIGS. 27A-27B). The repeat-low xenograft HCT-116 did not respond to either 3TC or AZA alone, but there was a trend of improved response to 3TC+AZA combination. Variability of repeat RNA expression between drug treated SW620 and HCT116 tumours was observed, indicating differing adaptive transcriptional responses to Repeatome modulators (FIG. 27C and FIG. 28). 79 genes highly anti-correlated with the HSATII repeat RNA (Pearson R≤0.75; FIG. 27D) that were found to be significantly enriched for chromatin factors (FIGS. 27D-27E and FIG. 28) were identified. This correlation is consistent with our in vitro RNA-seq analysis of cell lines treated with 3TC and AZA. Since AZA is administered clinically as a cytotoxic agent, other cytotoxic therapies may also induce repeat RNA expression or potentially be selected for in persister cells that are chemoresistant. To investigate this possibility, CRC cell lines were treated with the standard combination chemotherapy 5FU/Oxaliplatin (FOLFOX) for 14 days and detected marked elevation of HSATII RNA by RNA-ISH analysis (FIG. 27F and FIG. 29). HSATII RNA-ISH was then to 160 human primary CRC tumours that were untreated or pretreated with cytotoxic chemoradiation before resection, which demonstrated significant enrichment of HSATII repeat RNAs in tumours that received cytotoxic therapy (FIG. 27G). To determine if this had therapeutic implications, CRC lines were treated with 5FU/Oxaliplatin+/−3TC. These data showed significantly increased cytotoxicity in all 4 cell lines with the combination compared to 5FU/Oxaliplatin alone (FIG. 27H).

### Example 10: Clinical Trial of NRTI Effects on TP53 Mutant Metastatic Colorectal Cancers

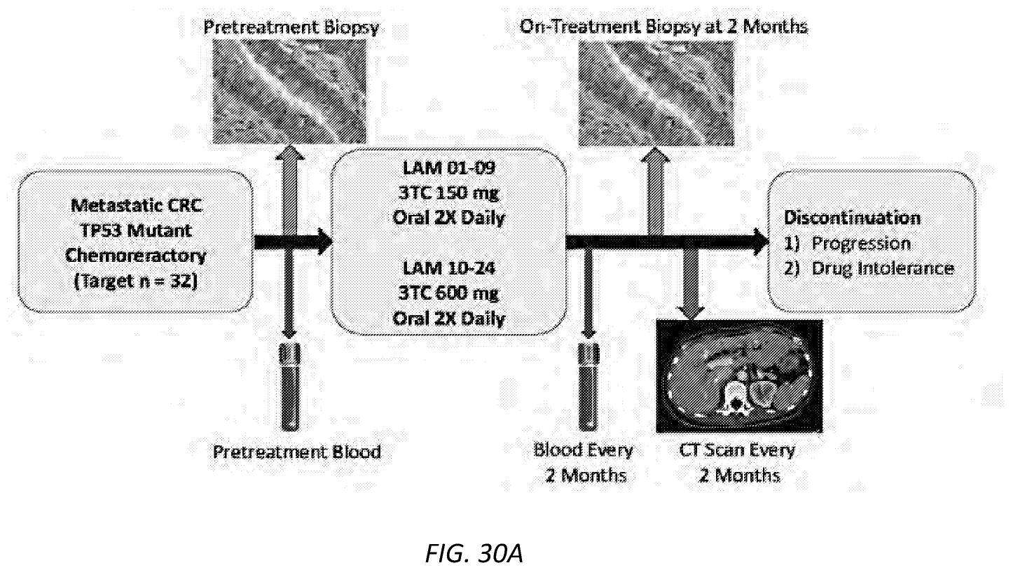

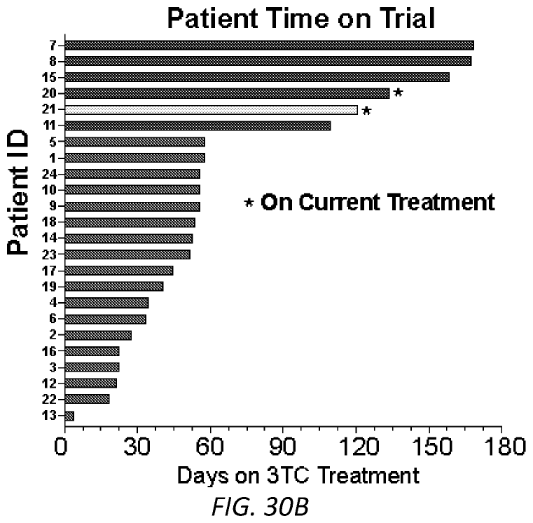

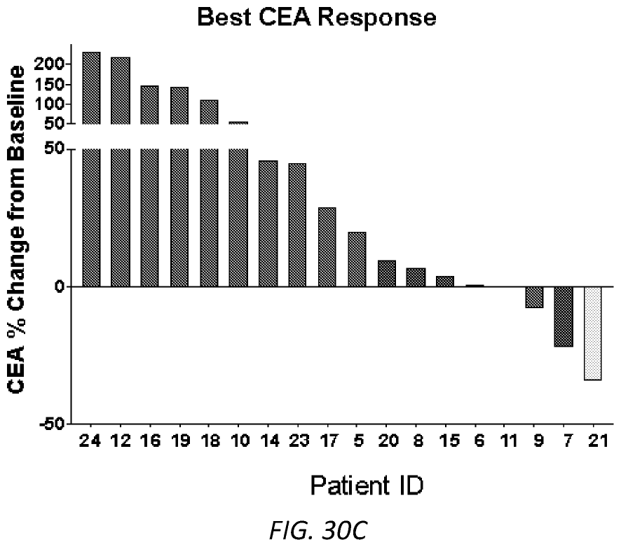

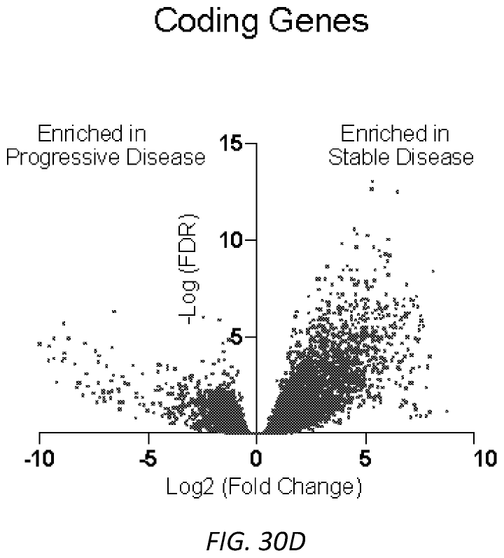

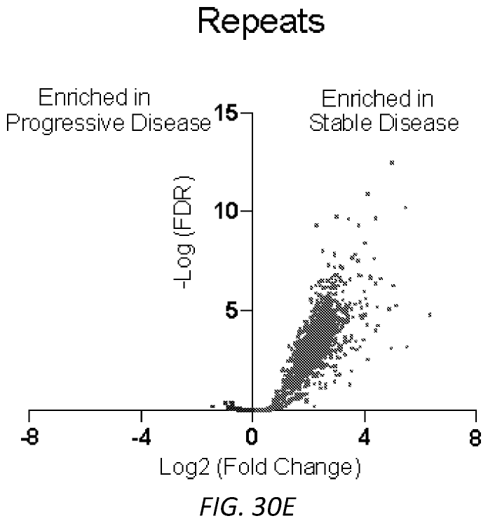

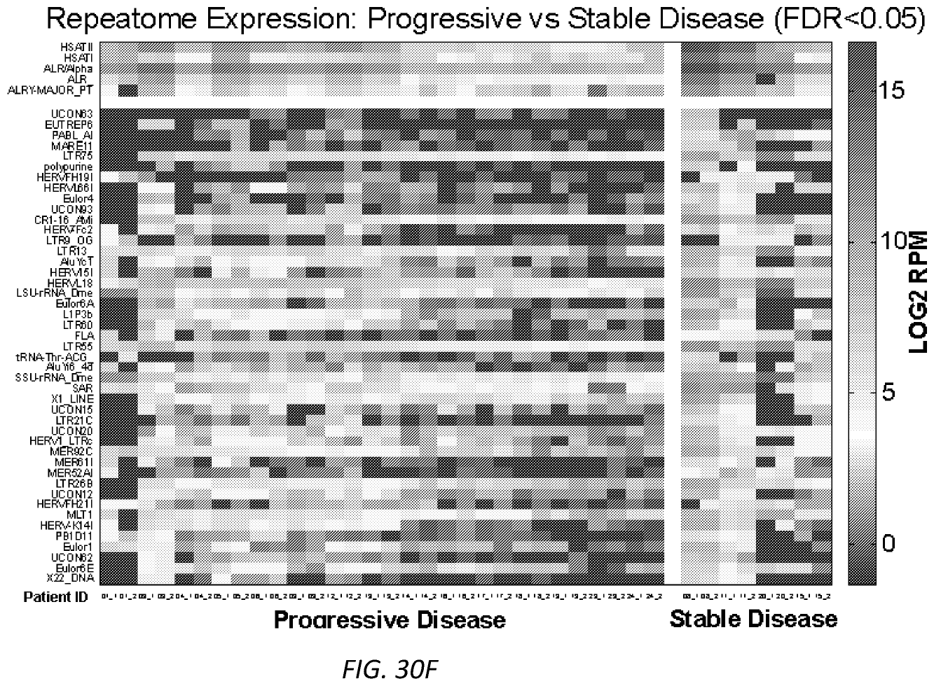

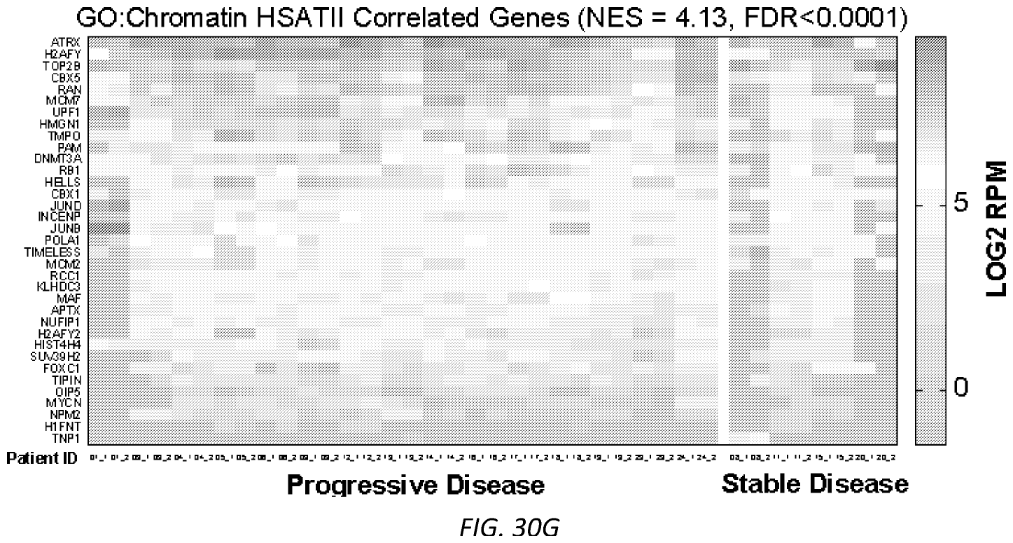

The data in Examples 7-9 provided preclinical evidence to support the initiation of a single-arm Phase 2 clinical trial (NCT03144804) of 3TC in patients who have progressed on systemic therapy for metastatic CRC with TP53 mutations (FIG. 30A). Here, 24 patients were treated with 3TC. The first 9 patients received 150 mg orally twice daily for 28 day cycles, the maximum FDA approved dose of 3TC for HIV. After indication of safety in the first 9 patients, an IRB amendment was made to increase dosing to 600 mg orally twice daily for 28 day cycles. Tumour assessments were performed every 8 weeks until documented disease progression by RECIST criteria or drug intolerance. The median age was 60 years (range 27-83) with 15 males and 9 females. A total of 5 of 24 (21%) patients (7, 8, 11, 15, 20) had stable disease on single agent 3TC with a median progression free survival of 159 days (FIG. 30B). Notably, one patient had mixed response with some reduction in tumour size of target lesions with concordant drop of CEA (−34%), but had new metastases (21). This patient elected to continue with 3TC treatment despite new metastases. At the time of data analysis, patients 20 and 21 were still on trial. The best response of the colon cancer serum marker carcinoembryonic antigen (CEA) was relatively unchanged (0-10%) or decreased from baseline in all patients with stable disease (FIG. 30C). Pre-treatment biopsies were obtained on 19 of 24 patients that were processed for RNA-seq in at least duplicate to evaluate the expression of repeat RNAs as well as genes potentially involved in response or resistance to 3TC treatment. Pre-treatment biopsies that were not obtained were due to safety for difficult biopsy locations or deferred by the patient. Differential expression of coding genes (FDR<0.05) from patients with stable disease (SD) compared to progressive disease (PD) on treatment revealed 1813 genes higher in SD and 421 genes higher in PD (FIG. 30D). Hypergeometric gene enrichment analysis of genes higher in SD noted significant overlap with the HALLMARK_INFLAMMATORY_RESPONSE gene set (FDR 0.023) suggestive of a relationship of tumour inflammation with clinical activity of 3TC. Similar differential expression analysis of repeat RNAs showed a striking disproportionate number of repeat RNAs expressed higher in SD compared to PD patients (FIG. 30E), with 1160 statistically significant repeats RNAs higher in SD with a FDR<0.05. Significantly higher HSATII (15 fold higher, FDR 4.86×10⁻⁷) and ALR (11.6 fold higher, FDR 7.85×10⁻⁶) repeats were found in patients with SD. Patients 8 and 11 tumours had the highest HSATII and ALR repeat RNA expression in this cohort and both had disease stability for 168 and 110 days, respectively (FIG. 30F). Moreover, HSATII correlation analysis with coding genes again demonstrated an inverse relationship with chromatin factors (FIG. 30G). Altogether, these transcriptional findings were consistent with our preclinical xenograft and in vitro models demonstrating a benefit of 3TC in patients with TP53 mutant CRC tumours with elevated repeat RNAs.

These early results are encouraging as they provide support for the use of NRTIs as a new class of anti-cancer therapeutic. The lack of significant dose limiting toxicity of 3TC in metastatic cancer patients also affords the use of 3TC in combination with existing cancer therapies in future clinical trials to augment the efficacy of these drugs as we have shown with 5FU/Oxaliplatin and AZA in colorectal cancer cell line models. Moreover, these results with single agent 3TC provides a foundation to evaluate combination reverse transcriptase inhibitors to obtain more potent effects of disrupting the cancer Repeatome, a strategy that clearly changed the course of HIV disease control. Without wishing to be bound by theory, these results also provide potential mechanistic insight into the recent work demonstrating an apparent decreased incidence of breast, prostate, and colorectal cancers in patients living with HIV who are on stable anti-HIV regimens that include NRTIs.

### Example 11: LINE-1 Expression as an Epigenetic Marker of Neoplastic Transformation of Barrett's Esophagus

Barrett's esophagus (BE), the metaplastic conversion of esophageal squamous to columnar mucosa, is an asymptomatic and prevalent condition, but of significant clinical importance given the increased risk for developing esophageal adenocarcinoma. Endoscopic surveillance programs have shown that early detection of neoplasia and intervention is important for BE patient outcomes. The histologic diagnosis of dysplasia is the gold standard for identifying patients at risk for cancer, but there continues to be relatively low inter-observer agreement related to subjective criteria and processing artifacts. These problems are accentuated for low-grade dysplasia with one study showing only 15% of BE cases with concordant diagnoses of low-grade dysplasia when reviewed by gastrointestinal pathologists. The optimal biomarker would also predict neoplastic progression prior to the advent of dysplasia and thus permit triage of BE patients to early interventions or modified surveillance programs.

Multiple genetic markers of dysplasia have been studied, but often these events are found in BE that never progress to dysplasia. Collectively, these sequencing studies have revealed that TP53 is the most commonly altered gene found in esophageal adenocarcinoma (69%) and its mutation is the earliest genetic event separating high grade dysplasia from non-dysplastic BE. In parallel, others have shown a combination of global genomic hypomethylation and frequent hypermethylation of specific gene promoters correlate with dysplasia. However, testing for TP53 mutations and using methylation arrays for diagnostic purposes in multiple biopsies is tedious and costly.

The LINE-1 (L1) repeat makes up 18-20% of the human genome and comprises 12% of all DNA CpG methylation sites, which has led to its use as a proxy of global genomic methylation. Interestingly, TP53 has been shown to regulate repeat expression across species, and therefore, L1 expression may be a marker of TP53 functional loss as well as other epigenetic modifiers. It was first determined whether L1 RNA expression is associated with TP53 function in esophageal cells using Het1A cells.

Het1A (American Type Culture Collection, Manassas, Va.), a human esophageal squamous epithelial cell line immortalized by the SV40 transfection was cultured in low glucose Dulbecco's Modified Eagle Medium (DMEM; Invitrogen, Carlsbad, Calif.), supplemented with 10% fetal bovine serum (FBS; Invitrogen) and 100 U/ml penicillin G and 100 μg/ml streptomycin (Invitrogen), at 37° C. in a humidified incubator containing 5% CO2. The cells were detached from the flasks before subculturing by the removal of the medium and the addition of 1 ml of 0.25% trypsin for 3 to 10 min.

At 70% confluence, cells were placed in serum-free DMEM for 24 hours before bile acid exposure. The Het1A cell lines was exposed to 500 M deoxycholic acid (DCA) (Sigma, St. Louis, Mo.), in serum-free medium for 24 h. Cells were harvested at the end of 24 hours with 0.05% trypsin solution (Invitrogen). Cells were exposed to 95% ethanol (for bile acid) solution as controls. All experiments were performed in triplicate.

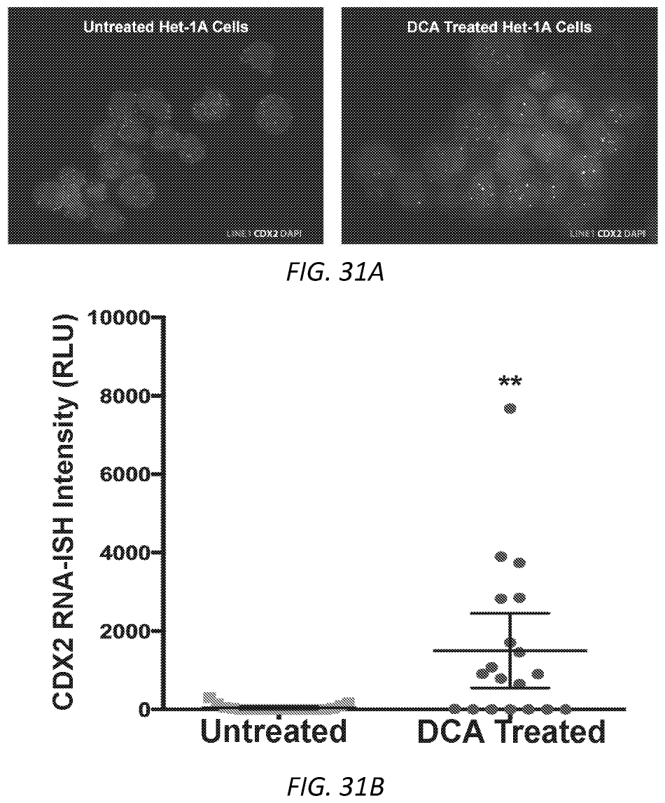

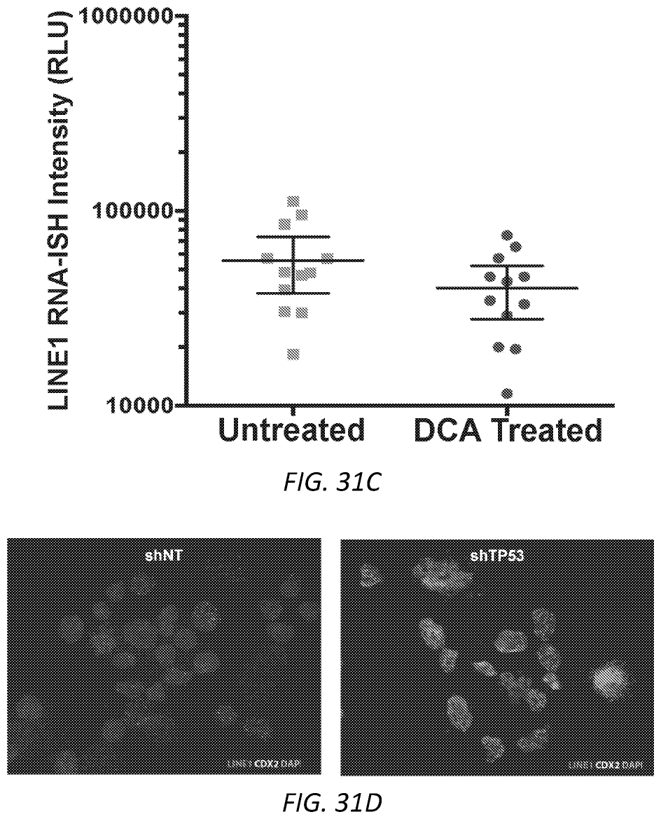

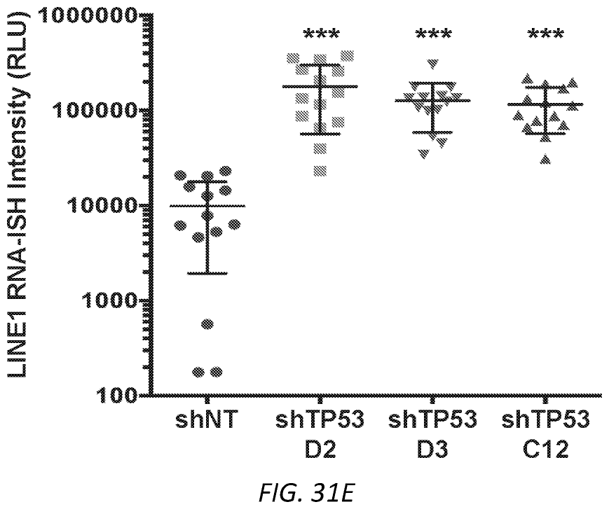

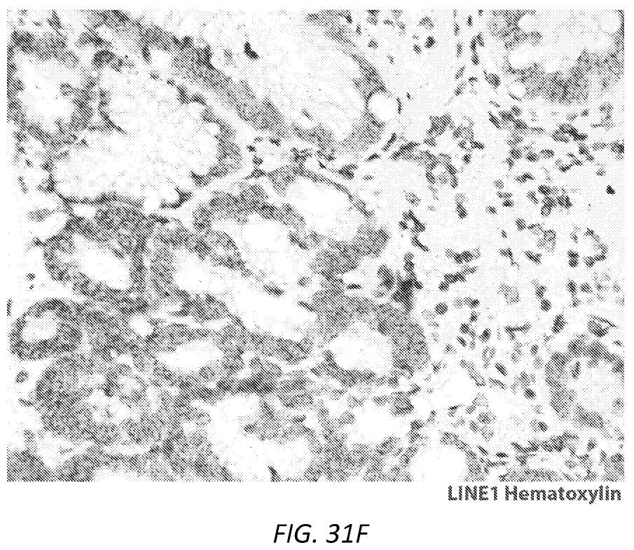

Using the normal esophageal cell line Het1A, metaplasia was induced in vitro using the bile salt deoxycholic acid (DCA). RNA-ISH was performed for CDX2 (a marker of metaplasia) and for L1 in Het1A cells treated with or without DCA (FIG. 31A). CDX2 was significantly induced with treatment with DCA (P=0.003), but there was no significant difference in expression of L1 (FIGS. 31B and 31C). To test the effect of TP53 suppression on L1, three different lentiviral shRNAs targeting TP53 and a non-target (NT) control were used to infect Het1A cells. In brief, Het1A cells were infected with pLKO-based lentiviruses encoding either scrambled shRNA or shRNAs targeting human TP53 (shTP53D1: TCAGACCTATGGAAACTACTT (SEQ ID NO:12); shTP53D2: GTCCAGATGAAGCTCCCAGAA (SEQ ID NO:13); shTP53D3: CACCATCCACTACAACTACAT (SEQ ID NO:14); shTP53C12: CGGCGCACAGAGGAAGAGAAT (SEQ ID NO: 15)). Cells were selected 48 hours post infection with 2 μg/mL puromycin for 7 days.

For analysis of TP53, cDNA synthesis was conducted using Invitrogen SuperScript III kit according to manufacturer's protocol. qRT-PCR was conducted using Fidelitaq polymerase and FAM labeled primers. Primers used were human TP53 (forward: 5′-AACCCACAGCTGCACAG-3′ (SEQ ID NO:16)); reverse: 5′-CCTTCCCAGAAAACCTACCAG-3′ (SEQ ID NO:17))); human GAPDH (forward: 5′-TGTAGTTGAGGTCAATGAAGGG-3′ (SEQ ID NO:18)); reverse: 5′-ACATCGCTCAGACACCATG-3′ (SEQ ID NO:19)). All qPCR assays were conducted in triplicate.

L1 expression was significantly higher in TP53 knockdown cells compared to control cells (P<0.0001; FIGS. 31D and 31E). To determine if these findings were concordant in tissue, 12 cases of BE with evidence of dysplasia (6 low grade, 6 high grade) and strong diffuse p53 reactivity by immunohistochemistry (IHC), a surrogate of p53 mutant protein, were stained with L1 RNA-ISH. Normal Het1A cells, DCA treated Cells, TP53 Knockdown cells and NT control cells were centrifuged onto poly-L-lysine coated glass slides (Sigma Life Sciences, P0425) for 5 minutes at 350 rpm using Fisher Double cytology funnel (Cat No: 10-356). Slides were dried for 10 minutes, fixed with 4% PFA for 20 minutes and washed with 1×PBS for 10 minutes before dehydration and storage in 100% ethanol at −20 degrees Celsius until staining procedure. ViewRNA ISH Cell Assay Kit (Affymetrix, Santa Clara, Calif.) was used to stain cell line slides. Cells were permeabilized using Detergent Solution QC for 5 minutes at room temperature (RT). RNA was unmasked using Protease QS (1:2000 dilution) for 10 minutes at RT. Type 1 probes for CDX2 (VA1-10441, Affymetrix, Santa Clara, Calif.) and Type 6 probes for LINE-1 ORF1 (VA6-16962, Affymetrix, Santa Clara, Calif.) were hybridized to target mRNA for 3 hours at 40° C. Signal amplification was achieved through sequential hybridization of Pre-Amplifier molecules, Amplifier molecules, and fluorescently conjugated Label Probe oligonucleotides. Cells were stained with DAPI (Invitrogen, D3571; 5 μg/ml) for 1 min at RT. Slides were scanned on the Keyence fluorescent imaging platform for quantification and analysis (Keyence Corporation of America, Itasca, Ill.). LINE-1 quantification was done on cell lines after ISH using the Keyence BIOREVO fluorescent microscope. Each slide was imaged in 10 different locations with DAPI, Cy3 (green), and Cy5 (red) filters. Channels were merged into a single image, which was then processed through the BIOREVO Analyzer Hybrid Cell Count. Individual cells were recognized by DAPI and cell boundaries were hand adjusted with the fine-edit tool. Red and green signal was detected for each cell above a uniform, automated brightness threshold. Signals in red and green color represent expression of LINE-1 and CDX2, respectively.

Using normal adjacent stromal cells as a baseline internal reference for L1 expression, 11 of 12 (92%) cases with abnormal p53 staining had concordant high L1 signal (FIG. 31F). Together, this data support a functional relationship of TP53 and L1 expression in esophageal epithelial cells that is independent of metaplasia.

Statistics for Example 11 were done by KSA using GraphPad Prism 5. Student t-tests were performed to compare median fluorescent intensities for LINE-1 and CDX2 RNA expression between all cell line conditions. The kappa statistic was used to test the interobserver variability. Values of 0.4-0.6, 0.6-0.8 and >0.8 were taken to reflect moderate, substantial or excellent correspondence, respectively. Analyses were performed using SPSS 21.0 (SPSS, Chicago, TL, USA).

### Example 12: In Vivo LINE-1 Expression as an Epigenetic Marker of Neoplastic Transformation of Barrett's Esophagus

To determine the utility of L1 RNA-ISH as a biomarker of dysplasia, we pursued a cross sectional study on a cohort of 109 esophageal biopsies with BE. The mean age of the cohort was 67 years with a male predominance (M:F ratio=3.7:1).

All samples were collected from archived tissues after clinical diagnostic use under an MGH IRB approved protocol 2013P001388. Formalin fixed paraffin embedded biopsies from consecutive cases diagnosed at this institution as negative for dysplasia (n=28), indefinite for dysplasia (n=18), low-grade dysplasia (n=11), high-grade dysplasia and (n=21) intramucosal adenocarcinoma (n=7) were identified. Nineteen patients with adenocarcinoma invasive into the submucosa were also evaluated.

All samples were re-analyzed for histological features of dysplasia by three experienced gastrointestinal pathologists (LRZ, AM, GRL) who were blinded to the L1 results. The original diagnosis on each case was recorded. The cases were originally evaluated by a pathologist with a subspecialist interest in gastrointestinal pathology With the exception of submucosal invasive carcinoma, the H&E stained slides were re-reviewed by 3 gastrointestinal pathologists (GRL, LRZ and TM). The reviewers were asked to categorize the cases into one of the following categories: 1) negative for dysplasia, 2) indefinite for dysplasia, 3) low-grade dysplasia, 4) high-grade dysplasia, and 5) intramucosal adenocarcinoma. A consensus diagnosis was judged achieved if 2 of the 3 reviewers agreed on a single diagnostic category.

ISH was performed using automated ViewRNA platform (Affymetrix) on 3 micron sections. This technology utilizes a branched DNA structure for signal amplification to enable detection of mRNA in formalin-fixed paraffin-embedded tissue. Automated ISH assays for Line1 ORF1 mRNA were performed using View-RNA eZL Detection Kit (Affymetrix) on the Bond RX immunohistochemistry and ISH Staining System with BDZ 6.0 software (Leica Biosystems). Paraffin-embedded full-faced (whole) tissue sections were processed automatically from deparaffinization, through ISH staining to hematoxylin counterstaining; sections were coverslipped off-instrument. Briefly, 3 micron thick sections of formalin-fixed tissue were baked for 1 hour at 60 C) and placed on the Bond RX for processing. The Bond RX user-selectable settings were as follows: ViewRNA eZ-L Detection 1-plex (Red) protocol; ViewRNA Dewax1; View-RNA HIER 10 minutes, ER1 (95); ViewRNA Enzyme 2 (20); ViewRNA Probe Hybridization. With these settings, the RNA unmasking conditions for the esophageal mucosal biopsies consisted of a 10-minute incubation at 95 C in Bond Epitope Retrieval Solution 1 (Leica Biosystems), followed by 10-minute incubation with Proteinase K from the Bond Enzyme Pretreatment Kit at 1:1000 dilution (Leica Biosystems). Human Line1 ORF1 (Cat #ZVA1-16742) and housekeeping mRNA-targeting probe sets composed of a cocktail of GAPDH, PPIB, and ACTB were diluted 1:40 in ViewRNA Probe Diluent (Affymetrix); the housekeeping gene sets were evaluated in selected cases. Post run, slides were rinsed with water, air dried for 30 minutes at room temperature, dipped in xylene, and mounted using Histo-Mount solution (Life Technologies, Grand Island, N.Y.).

L1 reactivity was detected in non-neoplastic stromal cells, lymphoid cells as well as normal squamous epithelium. High L1 in columnar epithelium was defined as increased signal in epithelial cells when compared to the adjacent stromal cells. In most cases well-defined nuclear and cytoplasmic dots were identified in both compartments, and to be judged as high L1, we required epithelial cells to show twice as many individual dots per cell. The L1 slides were reviewed by 2 observers (VD and KM), neither participated in the analysis of the H&E slides.

The consensus diagnosis was used as the gold standard and there was moderate agreement between pathologists (kappa 0.43-0.51). Esophageal adenocarcinoma was high for L1 RNA-ISH in 92% of cases. Remarkably, L1 RNA-ISH was high in 33 of 34 cases (97%) in BE (based on consensus read) with any level of dysplasia (see Table 1 below).

TABLE 1
L1 in situ hybridization in Barrett's esophagus and related neoplasia.
Data based on re-analysis by three gastrointestinal pathologists.

| | Negative for | Indefinite for | Low grade | High grade | Intramucosal | Invasive |
|---|---|---|---|---|---|---|
| [symbol] | dysplasia | dysplasia | dysplasia | dysplasia | carcinoma | adenocarcinoma |
| n = 96 | (29) | (9) | (17) | (17) | (5) | (19) |
| [symbol] L1 Low | 24 (83%)# | 4 (44%)## | 0 (0%) | 1 (6%) | 0 (0%) | 2 (11%) |
| [symbol] L1 High | 5 (17%)* | 5 (56%)** | 17(100%) | 16 (94%) | 5 (100%) | 17 (89%) |

No consensus could be reached on 13 cases of the original 109 cases in the cohort
#4 patients had prior history of dysplasia
##0 patients had prior/subsequent history of dysplasia
*3 patients had prior history of high grade dysplasia
**3 patients had prior history of dysplasia [symbol]
indicates data missing or illegible when filed

The majority of negative dysplasia cases were also low for L1 (24/29; 83%); 3 of the 5 cases high for L1 reported prior high-grade dysplasia. L1 RNA-ISH distinguished dysplastic from never-dysplastic BE with a sensitivity and specificity of 91% and 88%, respectively.

There were 18 cases originally classified as indefinite for dysplasia that were re-categorized with the exception of 3 cases where no consensus was achieved (See Tables 1 and 2 and Supplementary FIG. 1).

TABLE 1
The indefinite for dysplasia category (as defined by the
original pathologist) reanalyzed following evaluation by 3
gastrointestinal pathologists. History of dysplasia prior or
subsequent to the index biopsy is recorded in parenthesis.

| | Low L1 | | High L1 | |
|---|---|---|---|---|
| | Never | | Never | Prior/ |
| | dysplastic | Prior | dysplastic | subsequent |
| | cases | HGD | cases | HGD |
| Negative for | 2 | 2 | 1 | 2 (both prior) |
| dysplasia (7) | | | | |
| Indefinite for | 1 | 0 | 1 | 0 |
| dysplasia (2) | | | | |
| Low grade | 0 | 0 | 1 | 5 (prior 2, |
| dysplasia (6) | | | | subsequent 3) |
| No consensus | 1 | | | 2 (1 prior, 1 |
| (3) | | | | subsequent) |

Prior = histologic evidence of High grade dysplasia prior to the index biopsy
Subsequent = histologic evidence of high grade dysplasia following the index biopsy

TABLE 2
L1 results based on original interpretation

| | Negative for | Indefinite for | Low grade | High grade | Intramucosal | Invasive |
|---|---|---|---|---|---|---|
| | dysplasia | dysplasia | dysplasia | dysplasia | carcinoma | adenocarcinoma |
| N = 109 | (28) | (18) | (15) | (22) | (7) | (19) |
| Line 1 Low | 24 (86%) | 6 (33%) | 3 (20%) | 2 (9%) | 0 (0%) | 2 (11%) |
| Line 1 High | 4 (14%) | 12 (67%) | 12 (80%) | 20 (91%) | 7 (100%) | 17 (89%) |

All cases with a consensus diagnosis of low-grade dysplasia (6/6) were L1 RNA-ISH high. Interestingly, 11 indefinite cases had a prior or subsequent biopsy with histologically proven high-grade dysplasia and 9 of these were high for L1, which suggests that there may be an epigenetic field effect that can be seen in tissue that lacks unequivocal morphologic evidence of dysplasia. To evaluate this possibility, cases that had a consensus diagnosis of dysplasia or carcinoma with a well-preserved area of unequivocally non-dysplastic mucosa were analyzed. In 46% (10/22) of these cases, at least one fragment of non-dysplastic mucosa was high for L1. This is consistent with recent work showing L1 protein expression by IHC in esophageal cancer as well as normal esophageal epithelial cells, pointing towards a field effect in at-risk tissue.

To compare the results, L1 RNA-ISH and L1 ORF1p IHC on 19 BE cases demonstrating a concordance of 84% were performed. To assess the validity of the ISH assay, immunohistochemistry for L1 was performed on a subset of cases. And in an effort to assess the relationship between L1 and TP53, immunohistochemistry for TP53 was performed on a subset of cases. Immunohistochemical expression of the p53 and L1 was evaluated by deparaffinizing FFPE sections and subjecting them to antigen retrieval using the Leica Bond protocol (Leica Microsystems Inc., Buffalo Grove, Ill.) with proprietary Retrieval ER2 (ethylene diamine tetraacetic acid solution, pH 9.0) for 20 minutes. A mouse monoclonal antibody against p53 (7 ml p53 Bond RTU Primary Lot No PA0057 clone DO-7 K we need a clone) and a rabbit polyclonal L1 antibody directed against ORF1p 11 were utilized, and signal was detected by the Polymer Refine Kit (Leica Microsystems Inc.) on a Leica Bond Rx autostainer. The results are shown in Table 3.

TABLE 3
Comparison of immunohistochemistry and
in situ hybridization

| Diagnosis | L1 ISH | L1 IHC |
|---|---|---|
| negative for dysplasia | High | positive |
| negative for dysplasia | Low | positive |
| negative for dysplasia | Low | negative |
| negative for dysplasia | Low | negative |
| indefinite for dysplasia | High | positive |
| indefinite for dysplasia | Low | negative |
| indefinite for dysplasia | Low | positive |
| indefinite for dysplasia | Low | negative |
| indefinite for dysplasia | High | positive |
| low grade dysplasia | High | positive |
| low grade dysplasia | High | positive |
| low grade dysplasia | High | positive |
| low grade dysplasia | High | positive |
| low grade dysplasia | High | positive |
| high grade dysplasia | High | positive |
| high grade dysplasia | High | positive |
| high grade dysplasia | High | positive |
| high grade dysplasia | High | positive |
| high grade dysplasia | High | negative |

Discrepant cases of L1 ISH and IHC are bolded.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treating a subject with cancer comprising administering to the subject a reverse transcriptase inhibitor (RTI) and a DNA hypomethylating agent.

2. The method of claim 1, wherein the RTI is selected from zidovudine (ZDV), didanosine (ddI), stavudine (d4T), zalcitabine (DDC), lamivudine (3TC), abacavir (ABC), tenofovir disoproxil (TDF), emtricitabine (FTC), etravirine lobucavir, entecavir (ETV), apricitabine, censavudine, dexelvucitabine, alovudine, amdoxovir, elvucitabine, racivir, and stampidine.

3. The method of claim 1 or 2, wherein the RTI is 3TC.

4. A method of treating a subject with cancer in a subject in need thereof, wherein the cancer expresses high levels of HSATII RNA, the method comprising administering to the subject a therapeutically effective amount of a HERV-K reverse transcriptase (HERV-K RT) blocking agent.

5. The method of claim 4, wherein the HERV-K RT blocking agent is an inhibitory nucleic acid.

6. The method of claim 5, wherein the HERV-K RT blocking agent is selected from the group consisting of a locked nucleic acid (LNA) molecule, a short hairpin RNA (shRNA) molecule, a small inhibitory RNA (siRNA) molecule, an antisense nucleic acid molecule, a peptide nucleic acid molecule, a morpholino, and a ribozyme.

7. The method of claim 4, wherein the HERV-K RT blocking agent comprises a zinc finger nuclease system, a transcription activator-like effector nuclease (TALEN) system, a meganuclease system, a Cpf1 nuclease system, a CRISPR/Cas9 system, or a CRISPR/Cas13 nuclease system.

8. The method of claim 4, wherein the HERV-K RT blocking agent is selected from the group consisting of a nucleoside analog reverse transcriptase inhibitor, a nucleotide analog reverse transcriptase inhibitor, non-nucleoside reverse transcriptase inhibitor, and a combination thereof.

9. The method of claim 8, wherein the nucleoside analog reverse transcriptase inhibitor comprises lamivudine, abacavir, zidovudine, emtricitabine, didanosine, stavudine, entecavir, apricitabine, censavudine, zalcitabine, dexelvucitabine, amdoxovir, elvucitabine, festinavir, racivir, stampidine, or a combination thereof.

10. The method of claim 8, wherein the non-nucleoside reverse transcriptase inhibitor comprises lersivirine, rilpivirine, efavirenz, etravirine, doravirine, dapivirine, or a combination thereof.

11. The method of claim 8, wherein the nucleotide analog reverse transcriptase inhibitor comprises tenofovir alafenamide fumarate, tenofovir disoproxil fumarate, adefovir, or a combination thereof.

12. The method of claim 4, wherein the HERV-K RT blocking agent is a cytidine analog or a guanosine analog.

13. The method of claim 4, wherein the HERV-K RT blocking agent comprises an anti-HERV-K RT antibody.

14. The method of any one of claims 1-13, wherein the administering results in a reduction in tumor burden in the subject.

15. The method of any one of claims 1-14, wherein the administering results in the death of a cancer cell in the subject via necroptosis.

16. The method of any one of claims 1-15, wherein the cancer is an epithelial cancer.

17. The method of claim 16, wherein the epithelial cancer is pancreatic cancer, colorectal cancer, breast cancer, prostate cancer, renal cancer, ovarian cancer, or lung cancer.

18. The method of any one of claims 1-15, wherein the subject has Barrett's esophagus.

19. The method of claim 18, wherein the colorectal cancer comprises microsatellite instable (MSI) colorectal cancer or microsatellite stable (MSS) colorectal cancer.

20. The method of any one of claims 1-19, further comprising administering an additional therapeutic agent to the subject.

21. The method of claim 20, wherein the additional therapeutic agent is an immunotherapy agent selected from the group consisting of an anti-PD-1 antibody, an anti-PD-L1 antibody, an anti-CD137 antibody, an anti-CTLA4 antibody, an anti-CD40 antibody, an anti-IL10 antibody, an anti-TGF-β antibody, and an anti-IL-6 antibody.

22. The method of any one of claims 1-21, wherein the method comprises:
detecting a level of HSATII RNA in a sample from the cancer;
comparing the level of HSATII RNA in the sample to a reference level;
identifying a subject who has cancer that has levels of HSATII RNA above the reference level; and
selecting the identified subject for treatment with the HERV-K reverse transcriptase (HERV-K RT) blocking agent.

23. The method of any one of claims 1-22, wherein the cancer comprises a mutation in tumor protein p53 (TP53).

24. The method of claim 23, wherein the method comprises:
detecting a level of TP53 in a sample from the cancer;
comparing the level of TP53 protein in the sample to a reference level;
identifying a subject who has cancer that has levels of TP53 protein below the reference level; and
selecting the identified subject for treatment with the HERV-K reverse transcriptase (HERV-K RT) blocking agent.

25. The method of claim 24, further comprising administering a DNA hypomethylating agent to the subject.

26. The method of any one of claims 1-3 or 25, wherein the DNA hypomethylating agent is azacytidine, decitabine, cladribine, or a combination thereof.

27. The method of any one of claims 1-26, wherein the method comprises:
detecting a mutation in a TP53 allele in a sample from the cancer; and
selecting the subject for treatment with the HERV-K RT blocking agent.

28. The method of claim 27, wherein detecting a mutation in a TP53 allele in a sample from the cancer comprises:
determining a TP53 sequence in the sample and comparing the sequence to a reference sequence;
identifying a subject who has cancer that has a mutation in a TP53 allele; and
selecting the identified subject for treatment with the HERV-K RT blocking agent.

29. The method of claim 28, wherein detecting a mutation in a TP53 allele in a sample from the cancer comprises:
contacting the sample with one or more probes that specifically detect a mutation in a TP53 allele;
detecting binding of the one or more probes to the sample, thereby detecting the presence of a mutation in a TP53 allele in the cancer;
identifying a subject who has cancer that has a mutation in a TP53 allele; and
selecting the identified subject for treatment with the HERV-K RT blocking agent.

30. The method of any one of claims 1-29, wherein a sample of the subject expresses high levels of LINE-1 RNA compared to a reference sample.


SEQUENCE LISTING (19 sequences)

SEQ ID NO: 1 - DNA, 2109 bp, Artificial Sequence
  Y18890.14266-6374 Human endogenous retrovirus type K (HERV-K), gag, pol and env genes
atgatcccaaaagattggcctttaattataattgatctaaaggactgcttttttaccatccctctggcagagcaggattgtgaaaaatttgcctttactataccagccataaataataaagaaccagccaccaggtttcagtggaaagtgttacctcagggaatgcttaatagtccaactctttgtcagacttttgtaggtcgagctcttcaaccagttagagacaagttttcagactgttatattattcattattttgatgatattttatgtgctgcagaaacgaaagataaattaattgactgttatacatttctgcaagcagaggttgccaatgcaggactggcaatagcatctgataagatccaaacctctactccttttcattatttagggatgcagatagaaaatagaaaaattaagccacaaaaaatagaaataagaaaagacacattaaaaacactaaatgattttcaaaaattgctgggagatattaattggattcggccaactctaggcattcctacttatgccatgtcaaatttgttctctatcttaagaggagactcagacttaaatagtaaaagaatgttaaccccagaggcaacaaaagaaattaaattagtggaagaaaaaattcagtcagcgcaaataaatagaatagatcccttagccccactccaacttttgatttttgccactgcccattctccaacaggcatcattattcaaaatactgatcttgtggagtggtcattccttcctcacagtacagttaagacttttacattgtacttggatcaaatagctactttaattggtccgacaagattacgaataataaaattatgtggaaatgacccagacaaaatagttgtccctttaaccaaggaacaagttagacaagcctttatcaattctggtgcatggcagattggtcttgctaattttgtgggaattattgataatcattacccaaaaacaaaaatcttccagttcttaaaattgactacttggattctacctaaaattaccagacgtgaacctttagaaaatgctctaacagtatttactgatggttccagcaatggaaaagtggcttacacagggccaaaagaacgagtaatcaaaactccatatcaatcggctcaaagagcagagttggttgcagtcattacagtgttacaagattttgatcaacctatcaatattatatcggattctgcatatgtagtacaggctacaagggatgttgagacagctctaattaaatatagcatggacgatcagttaaaccagctattcaatttattacaacaaactgtaagaaaaagaaacttcccattttatattactcatattcgagcacacactaatttaccagggcctttgactaaagcaaatgaacaagctgacttactggtatcatctgcattcataaaagcacaagaacttcatgctttgactcatgtaaatgcagcaggattaaaaaacaaatttgatgtcacatggaaacaggcaaaagatattgtacaacattgcacccagtgtcaagtcttagacctgcccactcaagaggcaggagttaacccagaggtctgtgtcctaatgcattatggcaaatggatgtcacacatgtaccttcatttgggaagattatcatatgttcatgtaacagttgatacttattcacatttcatgtgtgcaacttgccaaacaggagaaagtacttcccatgttaaaaaacatttattgtcttgttttgctgtaatgggagttccagaaaaaatcaaaactgacaatggaccaggatattgtagtaaagctttccaaaaattcttaagtcagtggaaaatttcacatacaacaggaattccttataattcccaaggacaggccatagttgaaagaactaatagaacactcaaaactcaattagttaaacaaaaagaagggggagacagtaaggagtgtaccactcctcagatgcaacttaatctagcactctatactttaaattttttaaacatttatagaaatcagactactacttctgcagaacatcttactggtaaaaagaacagcccacatgaaggaaaactaatttag

SEQ ID NO: 2 - protein, 702 aa, Artificial Sequence
  CAB56603.1 Pol protein - Human endogenous retrovirus K
MIPKDWPLIIIDLKDCFFTIPLAEQDCEKFAFTIPAINNKEPATRFQWKVLPQGMLNSPTLCQTFVGRALQPVRDKFSDCYIIHYFDDILCAAETKDKLIDCYTFLQAEVANAGLAIASDKIQTSTPFHYLGMQIENRKIKPQKIEIRKDTLKTLNDFQKLLGDINWIRPTLGIPTYAMSNLFSILRGDSDLNSKRMLTPEATKEIKLVEEKIQSAQINRIDPLAPLQLLIFATAHSPTGIIIQNTDLVEWSFLPHSTVKTFTLYLDQIATLIGPTRLRIIKLCGNDPDKIVVPLTKEQVRQAFINSGAWQIGLANFVGIIDNHYPKTKIFQFLKLTTWILPKITRREPLENALTVFTDGSSNGKVAYTGPKERVIKTPYQSAQRAELVAVITVLQDFDQPINIISDSAYVVQATRDVETALIKYSMDDQLNQLFNLLQQTVRKRNFPFYITHIRAHTNLPGPLTKANEQADLLVSSAFIKAQELHALTHVNAAGLKNKFDVTWKQAKDIVQHCTQCQVLDLPTQEAGVNPEVCVLMHYGKWMSHMYLHLGRLSYVHVTVDTYSHFMCATCQTGESTSHVKKHLLSCFAVMGVPEKIKTDNGPGYCSKAFQKFLSQWKISHTTGIPYNSQGQAIVERTNRTLKTQLVKQKEGGDSKECTTPQMQLNLALYTLNFLNIYRNQTTTSAEHLTGKKNSPHEGKLI

SEQ ID NO: 3 - DNA, 21 bp, Artificial Sequence
  anti-HSATII S
cattcgattccattcgatgat

SEQ ID NO: 4 - DNA, 5 bp, Artificial Sequence
  HERV-K 5′ LTR and 3′ LTR
cattc

SEQ ID NO: 5 - DNA, 15 bp, Artificial Sequence
  scrambled non-targeting LNA
aacacgtctatacgc

SEQ ID NO: 6 - DNA, 16 bp, Artificial Sequence
  LNA1
gattccattcgatgat

SEQ ID NO: 7 - DNA, 21 bp, Artificial Sequence
  shRIPK3#1
ctgagagacaaggcatgaact

SEQ ID NO: 8 - DNA, 21 bp, Artificial Sequence
  shRIPK3#2
gtggctaaacaaactgaatct

SEQ ID NO: 9 - DNA, 21 bp, Artificial Sequence
  shRIPK3#3
gcactctcgtaatgatgtcat

SEQ ID NO: 10 - DNA, 17 bp, Artificial Sequence
  LNA targeting HSATII
  misc_feature (1)..(17): each residue is a locked nucleic acid
atggaatcatcatcgaa

SEQ ID NO: 11 - DNA, 17 bp, Artificial Sequence
  LNA3
  misc_feature (1)..(6): each residue is a locked nucleic acid
  misc_feature (7)..(7): 5-Methyl deoxyCytidine (iMe-dC)
  misc_feature (8)..(17): each residue is a locked nucleic acid
tggaatcatcgaatgga

SEQ ID NO: 12 - DNA, 21 bp, Artificial Sequence
  shTP53D1
tcagacctatggaaactactt

SEQ ID NO: 13 - DNA, 21 bp, Artificial Sequence
  shTP53D2
gtccagatgaagctcccagaa

SEQ ID NO: 14 - DNA, 21 bp, Artificial Sequence
  shTP53D3
caccatccactacaactacat

SEQ ID NO: 15 - DNA, 21 bp, Artificial Sequence
  shTP53C12
cggcgcacagaggaagagaat

SEQ ID NO: 16 - DNA, 17 bp, Artificial Sequence
  TP53 forward primer
aacccacagctgcacag

SEQ ID NO: 17 - DNA, 21 bp, Artificial Sequence
  TP53 reverse primer
ccttcccagaaaacctaccag

SEQ ID NO: 18 - DNA, 22 bp, Artificial Sequence
  GAPDH forward primer
tgtagttgaggtcaatgaaggg

SEQ ID NO: 19 - DNA, 19 bp, Artificial Sequence
  GAPDH reverse primer
acatcgctcagacaccatg